SEQUENCE LISTING (11 sequences)

SEQ ID NO: 1 - DNA, 512 bp, Rattus norvegicus
  modified_base (484): a, c, t, g, unknown or other
ttttttttttttttattgtatcataccaaagtttattgattacatcaaagaaaaatttctgtaatgaaaaaggcaagttgcattcataaaagatggcattcatgttcattttagaaagcaacaaagtagatgtaaaaaactgcttaagtgaaaaatgtaatatcgcagttccattttataagctgaaaaatgattttatcaacatttgcataaaatctgcactttatatactgcatgttattaaaaaattccaccactaaattatgacttttgcaaatttaggcttacatttatactgttgctggtgtatatgtagtagatatggaatggatatttttttgtttaataggcaacatccttaaacaatagacaacaatttggaaaattacagacattttgacagctcaaaaattattattcacatcatagcaatacggtcctactgttagatttcttgccatcttctgacataagagtagttaanatatagtgctaggaatgctggatggctc

SEQ ID NO: 2 - DNA, 413 bp, Rattus sp.
acaacaggttttattgggggggtggtcagtaacaggaatgtagttttcttttcaaaaagaaatcccattttctctgatttccccggtgcatttttccctgaacctgacatggggtacgtggtaagtagagaggaacttcgtataccaggttagaaatgaaatcccgagtggaaaaccttaatctttaaaaagaaaaaaaaaaaaggaaccaaaatattcaaatgcataaagtaagtgtagttgtcgtagttaaagctgtcactgtcaaagctggtcagagacacttacagctccaatccacgaacatttcagaatcctttcctaaaggctttcgggatggggtctgctgtgagccatctttcagggctccctctgggagcggattaacctctgaccatcagattagtggcttt

SEQ ID NO: 3 - DNA, 4349 bp, Rattus norvegicus
agagcttgggtgagctcccaggcaacccggagccacctccaaaaacaaaggggtaaggcacagacctttctctggcctagtgcttagtccggggtccgtggagcgtggctgtgggtctagaaaccctcggggcagctgcaggcttggaagaggcagggccgctctagcgccacccctgtctcctcccccgcaccgcccagcggcgcgaaactctggttggctcgccttgcaagtcatactttggttctctcattttgaaaactcctgacctcctcccgctccggccccatttggagaactcattgaagctgagtgttttgaggaccaggccagcttggagacggcaactctgttggacaccagcgggcaggaccggctccgtggcccgtgagcgtgggcgagatgcggagcaggagcaattctggggtccgcctggacgggtacgcgaggctggtgcagcaaaccatcctgtgttaccagaacccagtcactgggctgttatcagctagccatgaccagaaggatgcctgggtacgggacaacatctatagcatactggctgtttggggcctgggcatggcgtaccgtaagaatgctgaccgtgatgaggataaagccaaggcctatgaattggaacagaacgtagtgaagctgatgcgaggtcttctccagtgtatgatgaggcaggtggataaagtggagaagttcaagcatactcagagtaccaaggacagtctacatgccaagtacaacactgccacatgcagcactgtggtgggtgatgaccagtggggccacctccaggtggatgccacctccctcttcctcctgttcctggcccagatgactgcctcaggtttgcgtattatttttaccctcgacgaagtggccttcatacagaatcttgtcttctacatagaggctgcatataaagttgctgattatggaatgtgggaacgtggagataagaccaatcagggcattccagaattgaatgcaagctcagtgggaatggccaaggcagcactcgaagcaattgatgaactagatctttttggagcccatggaggacgcaaatcggtgatccatgtcctgcctgacgaagttgagcactgccagtcaattcttttctccatgttgccgagagcatcaacatctaaagaaatcgatgctggacttctttctattatttctttcccggcctttgcagtagaagatgtgcaccttgtgaatgtgaccaaaaatgaaattatttccaagcttcaggggcgttacggatgctgtcgcttccttcgggatggttataaaaccccaagagaggacccacaccgattgcattatgaccctgctgaacttaagctttttgaaaacattgaatgtgaatggcctgtgttctggacttatttaatcatagatggagtcttcaatggtgatgctgttcaggtccaggaataccgagaagccctggaaggaatattaatcagaggcaaagatgggatccacttggtgccagagctctatgccatcccaccagacaaggtggatgaagagtataagaatccacacacagtagaccgaattccactggggaagctgccccatctttggggacagtccttgtatatcctcagctcactgctagccgagggtttccttgccactggtgaaattgatccgttaaatagaagattttctacttcagtcaaacctgatgttgtagtccaagttgctgttttggcagaaaacagtcacattaaggggctcttgaagcaacatggaataaatgtccagagcattgctgatgtgcatccaattcgagtccagccaggccggattcttagtcacatatatgccaaattgggacaaaataagaatatgaaattgagtggtcgaccatatcggcacatcggggtccttggcacctctaaactctatgtgattaggaatcacatcttcacttttacaccccagttcactgaccagcatcacttctacctggccctggacaacgagatgattgtggagatgctgaggatcgaactggcctatctgtgcacctgctggcggatgactggccgccccactctcaccttccctgtcacacacaccatgctcacaaatgatggctcagacattcatcctgcagttctttctacaattagaaaactggaagatggctattttggaggtgctagagtaaaactaggaatcctggcagagttccttactacctcattctacacatacctgaccttcctggatccagactgtgatgagaagttgtttggtgacatcagtgataggagctttagtcctgacagtgagtcagacctgggaggataccttgaagacagcagtcatcaagaaagccaagatgaacttgaccagtatatcagtcaccttcttcaaagcacatccttgaagtgctacctgccccctctttgtaagaagtcagaagacagccatgttttcagtgctgtccactccactcgggacatactttctgtgatggccaaagcaaagggtttggaaactccattttttcccatgattttgccaactagagttctaagtggacaccgtaagtcactgaatcttgttgactcccctcagccattcctaaagaagactcctgaatatgactaccagtggcccagagatgaccaggatgaagtggactgtgagaagctagttgggcaactgaaagactgctcaaacctacaggaccaagcagacattctgtacattctttatgtaataaagggtccccgctgggataccaatttgtttggacagcatggagtcactgttcacagtcttctcggcgagctttatggaaaagctggcctgaaccaagaatggagtttgattcgctacatttcaggcctgctcaggaagaaagtggaggtcctggctgaggcctgtgcagatctgctgtcccaccagaagcagcttacagtgggcctgccccctgagccccgggagaagaccatatctacgcctcttcccccagaggagctcacagaactcatatacgaagccagtggacaggacatcagcattgctgtcctcacacaggagatcgtggtttacctggccatgtatgtccgggcccagcctagcctctttgcagagatgctcagactccggattggattgatcatccaggtgatggccacagagctggctcggagcctgaactgctcaggggaagaagcttcagagagcttgatgaacctcagccctttcgacatgaagagccttctgcatcatatactgagtgggaaggagttcggcgtagagagaagtgtgcgcccaatacactcctccatgtccagccctgccatctccatccatgaggtgggtcatactggagtcaccaaaactgagaggagtggtatcagcagactgaggagtgagatgaaacagatgaataggcgagctagtgctgatgagcagttctttccactgggccaagctgtgcccaaacgtttacattccatcaagtctgtgaggtccagcacaccgtcctccccgacaggcacatcatctacagactctggaggacaccacctgggctggggagaacagcagggccagtggctgcgcaggagaagattggatggggctatcaacagggtccctgtgggattctaccagagagtgtggaagatccttcagaagtgccatggtctatccatcgacggttatgtcctcccatcttcaacaacccaagagatgaccccttgtgagatcaagttcgctgtccatgtggaatcagtgctcaaccgtgtgtcccagcccgagtatcggcagctactggtagaagccatcatggtactgacactgctctcagatacggaaatggacagtatcgggggcatcatccatgtggaccagatagtgcagctagccaatcagctgttcctgcaggaccaggtgtctgatctggacagctcggccatcaaagtcacatagcagtagtgaagggaacacctgggcctatcacccctggctggaggcaggacacttggatgtttgttctcgttggaagtgcagctgctgagctgcagttgtcaggtcaccactggtgtccagggacagccccaccctgccccacaacacagtcttgagcttcaaggagctgtcacaaatcagtccatcaaagtctgggggagctgctgatgggccatacattcttcgtgaccacaaacccagccccattaggctttctggaccatgagtgacagcccatctggaacaagatgcaaatgattgtcacatcgttccttctaagcccctagccagctgggggatttaaaaataaagaattggaaccgaatcatggt

SEQ ID NO: 4 - DNA, 536 bp, Rattus norvegicus
catcacttaataaatttaataaattaagaggaagtctcacaaaactcagaagaatgtaacacacgggaataaggaatatgacaccagtttgcaaccattcacaaaaagtaccgtttgacaaaggaggaaaatcacaggaaataataatataaggcagtcttaaagagtatacatacataatagcacatcaaaatgatttgacagtaaattcaccacgtttacagttgtcacatgaactgttttaaaacatttataaaagtttgtagatttaacactgaactgtaagatttatttccaaatggttgtttttatatagatatacttcataccaaactgcacggaacttacgaatagcacacaagttgagaataattgctgtgttgggtactattacgtactactacttgatgacataagaattattaacaaatgtgctataagcgattatcattcaacttattgctagtgctttaggaactccatggtttatagtctatgtctctgtaaaacatatgctatgagcttgccctgatact

SEQ ID NO: 5 - DNA, 2063 bp, Rattus norvegicus
cagctccgtgagtatctgctctgttttctagatgccttcttgagcctgcttgtggccacccacagatacttgtaaggaggaaagaagtcagcttgccagagacactgtgaaatgagggattagaggccgggatccaagaaacaagagccgcagccaaaagacaagcgaacaggttccgaagatacttctactgagaggtctgccatggcctcccttggcgtccaactggtgggctacatcctaggccttttggggctattaggcacatcgattgccatgctgctccccaactggcgaacaagttcttatgttggtgccagcattgtgacggcggttggcttttccaagggcctctggatggagtgtgcgacccacagcactggcatcacccagtgtgatatctacagtactcttttaggacttcctgctgacatccaggctgcccaggccatgatggtgacatccagtgcaatgtcctcgctggcctgcattatctctgtggtgggcatgagatgcacagtgttctgccaggaatctcgagctaaggacagagtggctgtagtgggtggagtctttttcatccttggtggtatcctgggttttatcccagttgcttggaatcttcacggcattctccgggacttctactcaccactggtccctgacagcatgaaatttgagattggagaagctctgtacttgggaatcatttcagcgctgttttccttggtagctggagtcatcctctgcttttcctgctcacctcagggaaatcgtaccaactactatgatggctaccaggcccagcctcttgccactaggagctctccaagatctgctcaacagcccaaagccaagagtgagttcaactcatacagcctgactgggtatgtgtgaagaaccaggggccagagctgggggtggggtggggtggtggctgggactataaaaatagtagtggacaatgcccccagggttacaggcaaaggacattgccattgaatcatgtgtaaggtgctgctgagggtagaatgactttggccattggatgaagtaaaggcagagatgagaacaggggtgacagcatgtacatcgaattgccaaggacacttatcaagcgagcttttctatttcttttctcactaaggcaccttctgtgccctgaatgctcaacccaaaagtctaaatcccattgctgccactttttgctatgcccagaggctctcttctgccctttggtttacttggagatctgttccccaaccaatcatattccattgactaaccatctgtgatcaaaagaccttctccctctggctgaaattggctcttgactcaaatactaggggacagagagaagaaagaggggtggcttctgcgagaatgtttctagctcccttctcacatctccagccaaagaaatggattggtcagttataggccctgaagcctctcttccagttttgctatgagtgcaatatgtccagactggtttgggaaggaactgaaggaaagctatctcatggcatcccacagaaagcagctgagtataggatgggaggcaacctgggctccaaagaaacaaaaacaaacaacaaagcccaccagaatcacttctcagatcatctcctctaattgccacccatttgtgggtcaggaatggaagcactgctacaggaagacagtagaggcatacagaaagctgaaggctgcagccccctcactccaccgaggactggccaagaagctgaagtgaactaccgctcaaagtgctgtctcccactgaggtgtctgacccagcttcctgataaaccacaagacttggagccctagacaagggtttccttagggacaataaagcagtttttctagaactgccctttgacatgggagcagcagagggctaaaaggactctttagccctggccaaaaacgaatggggactttggtatggaagaactgctgggcagagatgcatggtaggtgtgtgtgctcctactgttttggagaggcaaccctaccagatactttagacttgttgggaaaaacaacctatctgggtc

SEQ ID NO: 6 - DNA, 3617 bp, Rattus norvegicus
cagaaagcctttctgcaacccaacgggagtggagccaggggctctggtcttcctgagatttggagcctggtgtccactgctaagaacacacagtgatgaaagtggaagagaaggctggggtcaaaaagctggagcccacaagctacaggaggagacacccagaacaagatttcccttcaatacatattggctttccagtccccggttactctcaaagaaaaagtgactcaaagggacatctttcaggtctgcaaagagtccagtggtccctgcaaccggacaaatctcagcaggacctggctggtccagacgggatcaaggccagcagcctgggaggctccgtggattttaccaaaaggattcggtctccagctgctgagcagctacaggacatcctgggggaggaagatgaggctcccaaccccaccctcttcacagagatggacactctacagcatgatggggaccagatggagtggaaggaatcagccaggtggataaaatttgaagaaaaggtagaggaaggaggagaacgctggagcaagccccatgtgtccacgctgtccctgcacagcctctttgagctccgcacttgcctgcagacagggacggtgctgctggatctggacagtggctctctaccacagatcatagatgatgtcattgagaagcagatagagggtggacttctgcgaccagagctccgggaaagggtcagctacgtgctcctgaggaagcatcgccaccaaaccaagaagcccatccaccgttcccttgcagacattgggaagtcggtttctactacgaatcgcagctctgcccggagcccctcagcaggcccaactctgcaccactccaccgaggacctgaggattcggcaaagcaccagctatggtcatctgtgtcatgcccagagtagaagcatgaatgacatctcccataccccaaacacagaccagcggaaaaacaaattcatgaagaagatccccaaggactcagaagcctccaacgtgcttgtgggcgaggtggacttcctggaccagccattcattgcgtttgttcgtcttgcccagtcttccatgctgggaggggtgaccgaggtgcctgtccccaccagatttctgttcatacttctaggaccttctggaagagcaaagtcctacaatgagattggccgagccattgcaaccctcatggtggatgatctcttcagcgatgtggcttataaagcacggaatcgagaagatctgatcgcaggagttgatgaatttctagatgaggtcattgtccttccccctggagagtgggatccaaacatcagaattgagccgcccaagaaagttccctcagccgacaagaggaaatctgtgttctctctggcagagctgggccagatgaatggctctgtgggcagaagcggagcctcggccggaggaggcggcagtggcggaggggctggaggcagcggggccggcggaggaggcagtggcaacgaggctgagatgccagctatgcatgaaattggggaggagctgatctggaccggaaggttttttggaggactgcgtctggatgtaaagaggaagctgccctggttcccaagtgacttctatgatggcttccacatccagtccatctctgctgtcctgttcatctacctcggctgtatcaccaatgctatcaccttcggtgggcttctgggggatgccacagacaattatcagggagtaatggagagcttcctgggcactgccatggctggcagcttgttctgcctcttctcggggcagcctctcatcatcctcagcagcacaggacccatcctcatctttgaaaagctcctctttgacttcagcaaggccaatggcctggactacatggagttccgcctctggattggtcttcactcagccatccaatgccttatcctggtggccacagatgccagctttatcatcaagtatatcacccgcttcaccgaagagggcttctccaccctcatcagtttcatcttcatctacgacgccatcaaaaagatgattggtgccttcaaatactatcccattaacacggacttcaagcccgactccatcaccacctacaaatgcgagtgcgtcgctcctgacacagtgaatacaaccactgtcaacgactcagccctgctggtgccaaacaccaacatgtctgtgtacaccccccttaacctcacagcgctggactggtctctgctgagcaagaaggaatgtctgagctatggggggcgcctgcttgggagctcctgccagttcgtccccgacttggccctcatgtccttcatccttttctttgggacttactccatgaccttgactctgaagaaattcaaattcagccgctatttccccaccaaggtccgaaccctggtggctgacttctccattgttttctccatcctgctgttctgtggaatcgatgcctgttttggcctgcaaacccccaagctgcacgtgcctaatgtcatcaagcccacaaggcctgaccgaggctggttcgttgccccctttgggaagaatccttggtgggtgtaccctgccagcatcctgccggccctgctggtgaccatcctgatcttcatggaccagcagatcactgctgtcattgtcaacaggaaggagaacaagctgaggaaggccgccggctaccatctggacctgttctgggtgggcatcctcatggccctgtgctccttcatggggctcccctggtatgtggctgccacggtcatctccatcgctcacatcgacagcctcaagatggagacggagaccagtgccccaggggaacagccccagttcctgggggtcagggagcagagagttactggagtcatggtcttcattctgacaggcatctccgtcttcctggctcctatcctgaagtacatccccatgccagtgctctatggtgtcttcctctacatgggtgtggcctccctgaatggcatccagttctgggaccgctgtaagcttttcctcatgccagccaagcaccagccggaccatgccttcctgcgccacgtgccccttcgccggatccacctgttcaccctggtgcagatcctctgcctggccctgctctggatccttaagtcaaccatggctgctatcatcttcccggtcatgatcctgggcctaatcattgttcgaaggctcctggacttgattttctcccagcatgacctggcctggattgacaatatcctcccagagaaggagaaaaaggaatcagacaggaagaagaggaggaaagaggtccacgagaacacggacaaggagccccagttccttcctccctcagttgtaaagatcccaatggaaggtatcccatctgatccccaaaacggtatccactgtgttggcagaaaaagatcttccagttggagccactctctctgactcctccttcagtgcctcagaacagatagaagcatcaccctgcatttcaaaatctcttgtccggcctcacctgctttcagctactcacggagtccactcttcatggtgccacaggtgaggatagagatggactctgacctcaacctcacagatgacgaggaggactatggataagaagaggtg

SEQ ID NO: 7 - DNA, 2252 bp, Rattus norvegicus
gaattcggcagagcttcgaaagagttgagaggcaagctgagttctttagccccacaccccagactgagcataaggattggggtccgggtgcgaatttagaagaagcacgaacgcttgaggaagagcccaaagaccttaagtcagggaaaagggacatagagtcttggggccctctggatttcgcatcaagcgaaacctttgcttccagagcccagagtgcggtgtcctctcggaggaggcacggcagcccctgcccctcacccctgagcggcgtcgaggaagggtgttttgaagggtgttttatcatggctgaggccacggagcccaaggaggtctcttcgggttcccaagggcaaccagaaggtgcggtgatcgagggaccaggagagcctggggctgcggacctggaggggcgggaagcgagcgagggagctgcagaggcacccagggacctgggggagggcgcggaggccatggcctcagggaaggaggaaggaggctgtgggcaggatggggaaatcggggaagtgcaggcgcaggacccgcgcccgggccccgggacggagacaccgggcaccagtggagctccgggtgaagcggaggctgcggaatgcgactccgagggcgccttgatcccgcagagcggaggaggcgccaagcgccagcaggtgcagggaacgagctcggggctagacgctcagggtgaggcccctgaggtccccgaggacgctcgccgggagccagaggaccccaaggcttcagaggcaggggaagaggcagaatctggtcaggaagctctgggaggcagcgcgccagagtcgcagatcaatccagaagtccagggacctgtaggggacaacatggacacagaggcccccgcaggagagccacaggggtcagagggagagccccagggtggaggggaaagcagccctcagccccaggatgaggcgattgagattgcagccgcagaggttggagggcacgagcccggggagctggcgggcgcttcagccgcagacgccaagggggaaggggaaaccctgaggaaagatgggttcgaggaagctgctcccgaggaggcaagagtagactctggcgagaatgggttcgaggaagcagctcccgaggaggcaagagtagactctggagagaatagggaccagggcaggctccaggaggagaccggggaggaagaagcgagaccggaatccgggttgaagggaccctgcgaggaggcgatccaagagaaggcccccgacggcagccttgatggagaggaggccaaatccaccgggcacgaggagtcgcaagtggagctcagtaaccacctagcggaggagaccagcgcccagggcggagaagagttggggcgagtgaatggacgtagggagaacggcccagcttcggaggagggggatctgggacaggagcacgacatcaccctctttgtcaaggctggctctgatggagagagcatcggaaactgcccattctcacagcgtctctttatgatcctctggctaaagggtgttatattcaatgtgacaacggtggacctgaaacggaagcctgcagacctgcagaacctggctcctgggaccaaccctcctttcatgacttttgatggtgaagtcaagacagatgtgaataagattgaggagttcttagaggagaagttagtccccccgaggtaccctaagctgggaacccaacaccctgaatccaactcagcgggaaatgacgtttttgccaaattctcagcgtttattaagaacaccaagaaggatgcaaatgacatttatgaaaagaacctactacgggccctgaagaagctggacagttacttaaacagcccgctgccagacgaaatagatgcctacagtacggaggatgtcactgtttcccagaggaagtttctggatggggatgagctcacactggccgactgtaacctcttacccaagctccacatcattaagattgtggccaagaaatacagaggttttgagtttccttctgaaatgactggcatttggcggtacttgaacaatgcttatgcccgggacgagttcactaacacgtgtccagctgaccaggagattgaacacgcgtattcagacgcagcgaaaaggatgaagtaatgctggcttgcgtctttcctcagatgagtgaacagggcttccaggaggaccttctatgtttcttccccaaaacagcttttgcaaaaagaacgctggactctctaaatg

SEQ ID NO: 8 - DNA, 300 bp, Rattus norvegicus
tttttttttttttttttaagggagcaacatgagtgatttggttgaacgtccttttatctttgaacagagattgggatgaatgaaatgacctcccccacgggaagaaagatgagctgtcaccgaaactgtaaagaatgtctctgttgcattcttcagaatatgctgtcaatgcttggttgcagtgatattcaaattccttagtagacccgacagcgatccagtgcgagcacactgggaacctgtccctgtggtcactgagacctactgtgtgcttctgtacgtgtgaaggggctttgaagt

SEQ ID NO: 9 - DNA, 591 bp, Rattus norvegicus
ttttttttttttttttatgtggtactttttatttggtctttaaactgtgcttcgtattaaatatgacctttcccaagtctcacagtatttctagttatttctgtgtaaacacactgtgacggcgagcttcagtgtcccctctaagtgagtaacttataaactcatgtcaacacccagccacctcatgtgctgcacctcagatcctatagctttctgtccagcaggtccacagccagggtaaagagaggctgggaaagaactgaaacaaaattaaaacatggccttgcctgtgttccgtgatgctatggaaacacatttaaagtttttttcatctcttttaatcaaataagcaaatcaagaaaggtcaaattctagggggttctatgctatcaggaaaaataaaaataatctgacttctatgactgaagaatgcaggacaccagggggtgcatggaagcaaaacacttgaaccagccaagaagcaaggtgccctgccaggcaattctctggtgagaagcccacgaaaggcatttatacctgagaacacacttctctccctgtctctgggtggagacatgagtgcccaaagtc

SEQ ID NO: 10 - DNA, 354 bp, Rattus norvegicus
tttttttttttttttttaaggtcaaaaccttttaattttcaaacatatattgagcaaaaaataattaaaaccctattagcagcccttttttcctagagggaaaaactacaaggtgtatatgtgggagggaacatgacagtgtgaggcgaagggcggcagagcttgtcctcagcttcttcataactaaacactaaaacagggatctcctgatctacagctttgaaaggccaaagactcagccctttcttgaagaactggggtcctcgcttctctggtattcgtgtatagagaggtggtatctctctatacctgaatgaacctgcctatgactggtagtccggtgcctgtaccctc

SEQ ID NO: 11 - DNA, 2279 bp, Rattus norvegicus
ccaaaggcagccagagaaccagtgtctcctgacagcctggtccctcaagagcaaagttcgcttctttgattggtggcttgtgaccggggagtggaccctcgggggtcttaggctatcactggaactccagaaagccatgcagagccttgagccatgaaggaatatgccgatgtcaccctctgtccagaggcagctgagctgagcaagacagagttctgcaatcctgctttcgacctggaagccgggccttcttgccctccaccagcattacagaaaaatgtcagcagcagcctccaaggtccctggcatgtccagcgtctccgggggctacaaccagactgccacttctccgggctctgtatcctgctgctcagcggcctgctgctactactgctggggctgctggtggctgtcatcctggctcagttgcaggctacatctctccccaggactaccaagaacccactgcccacccgaggaggcctcacccccacgggtgtcattcccagcaccacccctaacaccaccaccaccaccaccaccaccccagcaatcacagggcggcaagaggcagccatgagccctacacaccaaaccacctgtggaggcctccttcctggtcccagtggtttcttcagcagtcccaactacccagacctttacccaccccacagccactgcgtctggcatatccaggtagccacaggccagacaatacagctcaagattcaagtgctcagcacagaaagcatgctcgcctgtctttttgatcgcttggaaattagcccagcgcctacaggccctctgctcagaatgtgtggtaaaacacctcctgccacagttaacaccaacaccagtcacctccgtgtgtccttcgtctctgataacgatgtagaagggtctggtttccaggcctggtaccaagcagtggcccctggacattggagctgtacccactacgagttccactgtgacctgctcctctgcctgaagcaagactctgtgtgtgacggcattaaggactgcatcgatggcagtgatgaggccaactgcagtgccaagacgttggggtgtggagggaacctgactgagctctctggggtgttctctaccccaaactacccacagcactaccctcaccaacagctttgtacctggtatatcaaagtgcccgtgggatacaggataagactagagttccacaacttcagcctggaagaacaaactgaatgcaagtttgactacgtggaggtgtatgaggccagcaactcaggaacattcagctccctgggcaggttctgtggagcagagccaccatcccacctcgtctcctcgcagcaccagctgactgtaatcttcaagactgatcttggtatcagcagtgggggctttttagcaacctaccaggccatcaatacgacagagaacctttgtggacccagagagttctgccagagcagagtacgtagagaactgcaacggatttgtgacttatggaaagactgtgcaaatgacagcaatgacaactgcaacagccacttgtccccacaactggacctgacctgtgaacctgtccaggtggagatgtgccttggactaagctacaataccacagccttccctaacatctgggtgggcctggccacacagatggaggtgacagatatcctcagaggctacaagagcctgacaagtctaccctgctaccagactttccggaggttcctctgtgggctgcttgaacctcgatgcacctcactgggcactatcctacccccttgtcgttctgtctgccaggaggcagaacaacagtgccagtctagcctggcattactgggcatcccctggcctttcaactgcaacaggctgcctgtgccggctagcctggaagcttgctcccagccctgaccccaagccagcccccaccctctgtctgcccatcctcctctcccaagatgggtgggcaaggcaggcagaaaacaagggtggcctggacatgggactctgcccatcctctctggagcttctcaaagaggaaaaagaacagttattactctgatcctgcccaggactaacgggactgtcccatcatctttcccctaccgtcctcgagctcttatttgccaagacagccctggtaccattggacggcctagcagacttcctatccaagtctctgaccctgattccctgtcttctgcctccccccttttcatttaagttcaccatcagctgaccttcaagctttttcatgtagtcaataaaactgttttgagtga

(19) United States
(12) Patent Application Publication
(10) Pub. No.: US 2011/0165174 A1
(43) Pub. Date: Jul. 7, 2011

(54) Alternatively Transcribed Genes Associated with Memory Consolidation

(75) Inventors: Keith J. Murphy, Dublin (IE); Ciaran M. Regan, Dublin (IE); Robert H. Ring, Yardley, PA (US); David Von Schack, Arlington, MA (US)

(73) Assignee: University College Dublin, National University of Ireland, Dublin, Dublin (IE)

(21) Appl. No.: 12/867,378

(22) PCT Filed: Feb. 11, 2009
(86) PCT No.: PCT/US09/33732

§ 371 Date: Feb. 17, 2011

Related U.S. Application Data
(60) Provisional application No. 61/029,213, filed on Feb. 15, 2008.

(51) Int. Cl. A61K 39/395; C12Q 1/68; C40B 30/04; C07H 21/04

(52) U.S. Cl. 424/172.1; 435/6.1; 506/9; 536/23.1

(57) ABSTRACT

Methods of identifying gene targets associated with learning and memory and with memory consolidation, as well as to specific nucleic acid molecules that have been discovered to be associated with memory consolidation are provided. Accordingly, the invention also relates to methods of modulating learning and memory function, methods of diagnosing learning and memory disorders, and methods of identifying compounds that modulate learning and memory function via manipulations of the nucleic acid molecules and their products.

### FIELD OF INVENTION

This invention relates generally to learning and memory, and methods for detecting gene targets associated with these processes, methods of modulating learning and memory function, as well as to the gene targets themselves.

### BACKGROUND OF THE INVENTION

The term “memory” subsumes many different processes and requires the function of many different brain areas. Research in recent years has provided information necessary to understand many of the various components of memory and has identified associated brain regions. A newly acquired experience initially is susceptible to various forms of disruption. With time, however, the new experience becomes resistant to disruption. This observation has been interpreted to indicate that a labile, working, short-term memory is consolidated into a more stable, long-term memory.

Various mechanisms have been proposed to account for the formation of long-term memory. A wide range of observations suggest an evolutionarily conserved molecular mechanism involved with the formation of long-term memory. These include increased release of neurotransmitters, increased number of synaptic receptors, decreased KD of receptors, synthesis of new memory factors either in the presynaptic or postsynaptic element, sprouting of new synaptic connections, increase of the active area in the presynaptic membrane and many others. Synaptic plasticity, the change in the strength of neuronal connections in the brain, is thought to underlie long-term memory storage.

When acquired initially, newly learned information is held in a fragile state becoming robust over time (McGaugh et al., 2000). This consolidation process is driven by the hippocampus and requires de novo RNA translation and protein synthesis (Stork et al., 1999; Igaz et al., 2002), processes that provide the materials to allow the necessary structural changes in synaptic connections between neurons (Lamprecht and LeDoux, 2004). In the hippocampus, such synaptic plasticity involves three key phases: synaptic loosening, synaptic reorganization and synaptic selection. Synaptic loosening occurs when adhesive elements, such as neural cell adhesion molecule (NCAM), are selectively internalized and degraded synaptic curvature, size of synaptic elements and synaptic perforations (Marrone and Petit, 2002). For example, quantitative electron microscopic analysis of the rat hippocampus dentate gyms has revealed an increase in dendritic spine and synaptic density, 6-9 hours following either avoidance conditioning or spatial learning (O'Malley et al., 1998, 2000; Eyre et al., 2003). This increase in synaptic number is transient, however, returning to basal levels by 24-48 hours post-training These latter observations are indicative of a period of synaptic selection and elimination thought to be essential for the retention of only relevant connections in the memory circuit (Regan, 2004).

Several groups have used the large-scale screening of mRNA by microarrays to assess which genes may be involved in learning and memory at a transcriptional level (Cavallaro et al., 2002, D'Agata et al., 2003; Ressler et al., 2002; Leil et al., 2003; Igaz et al., 2004; O'Sullivan et al., 2007). These changes detected by microarray are usually considered indicative of transcriptional regulation but it must be noted that they could be due to altered RNA processing and post-transcriptional control (Mata et al., 2005). These studies have identified lists of genes that could be grouped into functional families including; transcription/translation, signal cascade enzymes, structural proteins and neural transmission. Although complex, these data begin to offer some insight into which genes may be involved in memory consolidation.

Despite continued efforts to develop methods to identify gene targets relevant for memory consolidation, and to identify genes and gene products important in regulating learning and memory processes, there is still a great need for methods that can detect such gene targets, so they can be utilized in screening therapeutics, in diagnosing learning and memory disorders, and in treating individuals with learning and memory disorders.

### SUMMARY OF THE INVENTION

Many diseases of the brain are underpinned by deficits in cognition-related synaptic plasticity. In the adult mammalian brain, the hippocampus is known to mediate the consolidation of contextual and spatial memories. Identifying mediators of learning and memory, and memory consolidation is essential for developing treatments for diseases or conditions associated with neurological impairment resulting in memory loss due to reasons such as Alzheimer's disease, senile dementia of the Alzheimer's type, senile dementia, brain trauma, age-associated memory impairment, amnesia, central nervous system ischemia, degeneration, Parkinson's disease, and stroke. Identification of such mediators is also essential for understanding mechanisms underlying synapse formation which may be useful for developing treatments or preventing one or more of the conditions stated above.

In accordance with these and other objectives, this invention provides methods for identifying gene targets associated with learning and memory, with memory consolidation, methods for modulating memory function, methods for diagnosing memory disorders, methods for identifying compounds that modulate memory function, as well as systems comprising one or more of these methods. Accordingly, this invention also provides isolated nucleic acid molecules, the molecules encoded by them as well as methods and compositions utilizing these learning and memory associated molecules to modulate various aspects of learning and memory.

In one aspect of the invention, a method is provided for identifying gene targets associated with learning and memory, and with memory consolidation. Transcriptional regulation is assessed over time in animals after the animals have been administered a first learning task. From this assessment, a cohort of transcripts is then detected from these animals. The cohort of transcripts has expression regulated in an identifiable temporal pattern following the first learning task. Another group of animals is administered a second learning task, and transcriptional regulation over time is assessed in animals after this second task. Following this second learning task, a cohort of transcripts is detected, that cohort having expression regulated in an identifiable temporal pattern following the second learning task. The identifiable temporal pattern of regulated expression from the cohort detected following the first learning task is then compared with the identifiable temporal pattern of regulated expression following the second task. Based on this comparison, a new cohort of transcripts is detected, and this cohort of transcripts is regulated in an identifiable pattern following each task. The new cohort of transcripts detected contains at least one transcript in common from the cohorts detected after each task, though it may contain many transcripts in common, or all transcripts in common. The identifiable pattern following each task may be a similar pattern, or may be a different pattern. Transcripts which are identified to be regulated in both cohorts are identified as being targets associated with learning and memory and memory consolidation.

Systems for carrying out all of the methods described herein are also provided, and optionally comprise a computer system and related software. For example, as relates to a plurality of learning, memory, and/or memory consolidation tasks and a computer system comprising software, said computer system configured to (a) detect a cohort of transcripts of which expression is regulated in an identifiable temporal pattern following at least two of the learning tasks; (b) compare the identifiable temporal patterns of regulated expression from the cohorts detected in (a); and (c) detect a cohort of transcripts from (b) wherein said transcripts are temporally regulated in tissue from animals administered a different learning, memory, and/or memory consolidation task, and which cohort comprises transcripts present in (b), which exhibits an identifiable temporal pattern of regulated expression following at least two of the learning tasks. Optionally, the computer system may output a result from (c) which is indicative of gene targets associated with learning, memory, and/or memory consolidation.

In a further aspect of the invention, a method is provided of identifying gene targets associated with learning and memory, and memory consolidation. Transcriptional regulation over time is assessed in animals after a plurality of learning tasks. The animals are comprised of at least two groups, and each group of animals is administered a different learning task. A cohort of transcripts is detected from each group. The cohort from each group has expression that is regulated in an identifiable temporal pattern following the learning task that each group is exposed to. The temporal identifiable pattern of regulated expression from each cohort detected following each learning task is then compared. Based on this comparison of cohorts, a new cohort of transcripts is detected, and this cohort of transcripts is regulated in an identifiable, temporal pattern following each task. The new cohort of transcripts detected contains at least one transcript in common from the cohorts detected after each task, though it may contain many transcripts in common, or all transcripts in common. The identifiable pattern following each task may be similar patterns, or may be different patterns. These regulated transcripts are also identified as gene targets associated with learning and memory and memory consolidation. Systems for carrying out the foregoing methods are also provided, as described above.

By following the temporal expression pattern according to the methods of this invention, transcripts may be identified having a high probability of being associated with learning and memory, and memory consolidation. Different patterns of expression may be informative. Thus, common expression patterns may suggest the contemporaneous involvement of a plurality of gene products, whereas sequential expression may suggest a dependent on, the expression of more earlier expressed gene products. The time points at which certain genes are regulated may also be informative of the specific mechanisms important for learning and memory processes.

An additional aspect of the invention provides isolated nucleic acid molecules identified according to the methods of this invention. In one embodiment, this invention comprises a nucleic acid sequence selected from the group consisting of SEQ ID NO:1 through SEQ ID NO:11.

In yet another aspect of the invention, a method is provided of modulating memory function in an animal. Memory function is modulated by enhancing or reducing the gene expression product of at least one nucleic acid sequence identified to be associated with learning or memory consolidation according to the method of this invention. Accordingly, through the analysis of temporal expression patterns, gene targets are identified that may serve as targets for modulating memory either by increasing or decreasing the function of the gene target. In certain embodiments of this invention it may desirable to increase memory function, whereas in other embodiments decreasing memory may be desired. Included among the gene targets for modulating memory are nucleic acid sequences selected from the group consisting of SEQ ID NO:1 through SEQ ID NO:11.

In another aspect of the invention, a method is provided of modulating memory function in an animal. Memory function is modulated by enhancing the gene expression of a target or targets identified according to methods of the invention, and may include at least one nucleic acid sequence selected from the group consisting of SEQ ID NO:1 through SEQ ID NO:11.

In yet another aspect of the invention, a method is provided of modulating memory function in an animal, which comprises reducing the gene expression product of a target or targets identified according to methods of the invention, and may include at least one nucleic acid sequence selected from the group consisting of SEQ ID NO:1 through SEQ ID NO:11.

In yet another aspect of the invention, a method is provided of modulating memory function in an animal, which comprises downregulating the gene expression of a target or targets identified according to methods of the invention, and may include at least ID NO:11.

In an additional aspect of the invention, a method is provided of diagnosing a memory disorder in an animal. The memory disorder is diagnosed by determining the gene expression profile of at least one nucleic acid sequence identified from the temporal expression pattern detected according to the methods of this invention, following, or throughout a learning and memory task. Examples of such nucleic acid sequences are those selected from the group consisting of SEQ ID NO:1 through SEQ ID NO:11. The gene expression profiles of normal and test subjects can be compared, and through this examination, it can be determined what nucleic acids or their products are not responding normally to tasks that elicit learning and/or memory processes.

In an additional aspect of the invention, a method is provided of identifying a compound that modulates memory function. The compound can be identified by determining the gene expression profile of at least one nucleic acid sequence identified from the temporal expression pattern detected according to the methods of this invention, following or throughout a learning and memory task. Examples of such nucleic acid sequences are those selected from the group consisting of SEQ ID NO:1 through SEQ ID NO:11. Compounds that increase or decrease the activity of these targets can be identified, which would be useful in modulating different aspects of learning and memory function.

These and other aspects of the invention may be more clearly understood by reference to the following detailed description of the invention and the appended claims.

### BRIEF DESCRIPTION OF THE FIGURES

FIG. 1: Expression of unknown genes demonstrated by the microarray studies to have common temporal patterns with each other and with known genes. In particular, midkine has been shown to mediate cognition-enhancement. The unknown ESTs were selected on the basis of their common pattern in both tasks and the fact that this pattern matches that of known genes.

FIGS. 2A-2C: Learning-specific increases of midkine in message and protein levels following water maze and passive avoidance learning. Panels A and B: Midkine mRNA change was demonstrated by microarray and validated by quantitative real-time PCR analysis of RNA samples extracted from hippocampal dentate gyms at increasing times post-water maze and passive avoidance training, respectively. In these studies, at each post-comparison of trained animals to passive controls. Panel C: The corresponding change in protein level was demonstrated following avoidance conditioning by standard western immunoblotting techniques. N: naïve; P: passive control; T: trained.

FIGS. 3A-3B: Midkine enhances spatial learning and memory. Panel A: At a dose of 2 μg, administered 2 h post-training on each of the four days of training, midkine improves spatial learning and was statistically significant on session three (Two-way ANOVA, p<0.05 for treatment). Panel B: In addition, Midkine treated animals exhibited superior memory of the platform location when tested 24 hours following the fourth and final training session (Student's t-test; p<0.05).

FIGS. 4A-4B: Midkine enhances recall of an odor reward association. Panel A: rats rapidly acquire an association between an odor and a food reward. Panel B: Subsequent treatment with Midkine (2 μg, ICV), 2 h post-training, results in significantly improved odor-reward association recall 96 hours post-training (Student's t-test; p<0.05).

FIG. 5: Midkine can reverse isolation rearing-induced sensory processing deficits. Isolation reared animals exhibit a significant deficit in prepulse inhibition (PPI) of startle. Four daily ICV injections of Midkine (2 μg/day; filled circles) reversed PPI behavior back to normal compared to vehicle-treated animals (open circles) (Two-way ANOVA, p<0.05 for treatment).

FIG. 6: The concordance between the transcriptional profiling studies and the proteomics analysis. Panel A reports the mRNA expression profile for transthyretin obtained from the water maze learning study. The mRNA increase for transthyretin at 2 h was seen to translate into a protein level up-regulation 6-12 h following learning (panel B). Intervention studies were carried out where transthyretin was administered ICV to animals trained across 4 daily sessions of water maze. Panel C reports the enhanced speed of acquisition of the spatial task conferred by transthyretin.

FIGS. 7A-7B: Unknown 8 (1397185_at; Klotho NAT) shows learning-specific increases at RNA level following water maze and passive avoidance learning that match those of Midkine Panels A and B: Unknown 8 RNA change was demonstrated by microarray and validated by quantitative real-time PCR analysis of RNA samples extracted from hippocampal dentate gyms at increasing times post-water maze (2-hr shown in FIG. 7A) and passive avoidance training (0.5-hr shown in FIG. B), respectively. In these studies, at comparison of trained animals to passive controls.

FIG. 8: Unknown 8 (1397185_at; Klotho NAT) expression is highly restricted to the hippocampus and cerebellum of the adult rat brain. In situ hybridization studies show that 1397185_at is expressed in a very discrete manner in the rodent brain. Panel A shows high expression in the hippocampus and cerebellum using the antisense probe while no such expression is seen with the sense control probe (Panel B). CA: CA pyramidal cells; DG: dentate gyms granule cells; CB: cerebellum.

### DETAILED DESCRIPTION OF THE INVENTION

This invention relates to methods of identifying gene targets associated with learning and memory processes and with memory consolidation. This invention also relates to nucleic acid molecules and their products which are identified according to the methods of this invention for identifying gene targets associated with learning and memory consolidation. Among such inventions are specific nucleic acid molecules that have been discovered to be associated with learning and memory processes and memory consolidation. Accordingly, the invention also relates to methods of modulating learning and memory function, methods of diagnosing memory disorders, and methods of identifying compounds that modulate memory function via manipulations of the nucleic acid molecules and their products.

An object of the invention is to provide a method of identifying gene targets associated with learning and memory processes. The inventors have observed that particular patterns of gene expression following exposure to learning and memory tasks are predictive of certain genes or gene fragments being involved in mechanisms relating to learning and memory.

The terms “learning” and “memory” subsume many different processes and require the function of many different brain areas. Memories are processed sequentially through several different phases that appear mechanistically and can be anatomically distinct. As contemplated by the invention, memory can include short-term memory and long-term memory. Short-term memory is rapidly formed and can last for seconds, minutes, hours, or days, and long-terms memory generally lasts from hours to days, weeks, or even years. Also contemplated by the invention, memory can include working memory, declarative (explicit) and procedural (implicit) memory. The terms “learning” and “memory” are often used in the their use in the art. As such, as used herein, these terms each apply equally to all aspects of the invention, and may be used interchangeably.

Any detectable change that one of skill in the art can measure which represents a change in any learning or memory processes is encompassed by the invention. The methods of this invention are generally useful for detecting spatial and temporal expression of genes and gene products associated with various aspects of learning and memory, including, but not limited to memory acquisition, consolidation, reconsolidation, and storage. Accordingly, embodiments of the invention may relate to any aspects of learning and memory. In certain embodiments, this invention relates to gene and gene products associated with memory acquisition, in other embodiments with memory storage, in yet other embodiments with memory consolidation, and in other embodiments with memory reconsolidation. As used herein the term “memory consolidation” means the process by which learned information is transformed into stable modifications. In some embodiments, the invention relates to memory re-consolidation. As used herein, the term “memory re-consolidation” relates to the process of reactivating consolidated memories, returning them to a labile state, so that they then may go through an additional consolidation process which may trigger molecular events similar or different from the original consolidation.

In one embodiment of the invention, a method is provided for identifying gene targets associated with learning and memory, or memory consolidation in which transcriptional regulation is assessed over time in a group of animals after the animals have been administered a first learning task. A cohort of transcripts demonstrating regulated expression in an identifiable temporal pattern is detected from this group of animals associated with a first task.

An additional group of animals is then administered a second learning task, which may be the same task or a different task, and a cohort of transcripts exhibiting regulated expression in an identifiable temporal pattern is then identified from this group of animals associated with the second task. This second cohort of transcripts is detected in the same manner as the first cohort. In some embodiments, the same group of animals is administered both tasks.

In one embodiment of the invention, more than two learning and memory tasks, or a plurality of learning and memory tasks, are utilized, and a separate group of animals is administered more than one of the tasks, or is administered all of the tasks. A comparison of all the identifiable temporal patterns is then made, to yield one cohort of transcripts that is regulated following the learning and memory tasks, containing transcripts that are related to learning and memory processes.

The identifiable temporal pattern of expression after the first task is then compared with the temporal pattern of expression after the second task, and if a plurality of tasks are used, then the identifiable temporal patterns of expression associated with all of the tasks are compared.

The comparison of the patterns of gene expression associated with each task includes determining which transcripts are regulated in an identifiable pattern following two learning and memory tasks or a plurality of learning and memory tasks. A new cohort of transcripts which exhibits an identifiable temporal regulation of expression following each task is then detected as being important for learning and memory processes. In one embodiment, the new cohort of transcripts includes at least one transcript which is common to both tasks, or which is common to a plurality of tasks. In another embodiment, more than one transcript is common to both tasks, or common to a plurality of tasks, and in yet other embodiment, all of the transcripts are common to both tasks, or common to a plurality of tasks. Also encompassed by the invention are cohorts of transcripts which include only a portion of the transcripts that are common to both tasks, or common to a plurality of tasks.

The identifiable patterns that are detected following the two or more tasks may be similar across tasks, having similar patterns, or the patterns may be inverse, and in some embodiments, they may have no relation to one another. Depending on the tasks utilized and the patterns assessed, the similarity or differences based on the comparisons of the detected identifiable patterns may be informative of the role that the regulated genes are playing in learning and memory processes. For example, without being bound by theory, regulated gene expression may show a trough in its pattern at a particular time point (or time points) following a task that requires an animal to avoid something, as in a passive avoidance task, and regulated gene expression may peak at a particular time point (or time points) following active learning, such as the Morris water maze task.

Transcriptional regulation can be assessed following the administration of the learning and memory tasks, although in some embodiments, groups of animals may be animals may also be assessed for transcriptional regulation at different points throughout the learning and memory task. These time points may be useful to serve as baseline measures, as well as for measuring processes taking place earlier in learning and memory formation. Such time dependent measurements provide for temporal assessments of transcriptional events during learning and memory processes and memory consolidation.

By an “identifiable pattern,” it is meant that of the gene transcripts assessed, at least two of the time points assessed exhibit a change in gene expression in the same direction, up-regulated or down-regulated, though in some embodiments, one or more of the time points assessed can reveal no change in gene expression. In a preferred embodiment, of the gene transcripts assessed, and considered to be in an “identifiable pattern,” three or more of the time points assessed exhibit a change in gene expression in the same direction, up-regulated or down-regulated, though in some embodiments, one of the time points assessed can reveal no change in gene expression. Any detectable change in amplitude of gene expression is included in the meaning of identifiable pattern. The change in amplitude of gene expression may be a change of 1%, 10%, 50%, or 100%, and in some embodiments the change in amplitude of gene expression may be 5-fold, 10-fold, 20-fold, 50-fold, 100-fold or greater. In some embodiments, no change in gene expression at one or more of the assessed time points may be meaningful, and can be encompassed as part of the identifiable pattern, and is within the scope of this invention. The change in expression can be either an increase or a decrease from baseline expression, or it may be an increase or decrease in gene expression relative to the gene expression at other time points within the temporal pattern. The gene transcripts exhibiting an identifiable temporal pattern are indicative of gene targets that are associated with learning and memory function. Compounds that increase or decrease the activity of the genes, gene fragments, and gene products identified as targets associated with learning and memory function may also be drug candidates for modulating learning and memory.

Changes in gene expression over any period of time are within the scope of this invention and may vary greatly depending on the species that is evaluated. In one embodiment, the gene expression is regulated within 24 hours of the learning and memory task. In another embodiment, the gene expression is regulated one hour, two hours, four hours, six hours, or twelve hours after the learning and memory task. In another embodiment, the gene expression is regulated within one week of the learning and memory leaning and memory task. In yet another embodiment, the gene expression is regulated within two months of the learning and memory task. In some embodiments the gene expression is regulated within one year of the learning and memory task, within five years of the learning and memory task, or within ten or more years of the learning and memory task. In some embodiments, the regulation of gene expression is regulated at time points throughout the administration of the task or tasks, and in some embodiments, the gene expression is regulated prior to the administration of the task or tasks.

In some embodiments of the invention, transcriptional regulation is assessed from brain tissue. Many regions within the brain that are important for regulating learning and memory functions, including but not limited to the hippocampus, the cortex, prefrontal cortex, neocortex, amygdala, striatum, and cerebellum can be assessed for transcriptional regulation prior to, during, and after learning and memory tasks. It is also contemplated by the invention that transcriptional regulation can be assessed from blood, plasma, lymph, urine, mucus, sputum, saliva, or tissue from other organs of the body.

In one embodiment of the invention, regulated transcripts are detected following a learning and memory task, which correspond to genes that have been previously identified to be associated with learning and memory processes. The identifiable pattern of temporal gene expression of the previously identified gene or genes is then compared with the other transcripts, those transcripts which are unknown, to yield a cohort of transcripts that is regulated in an identifiable temporal pattern with the gene or genes previously identified to be associated with learning and memory. In one embodiment, the average temporal pattern of the genes previously identified as associated with learning and memory is used as a “seed pattern” and all regulated genes are ranked for their similarity to this pattern based on the time points and amplitude as described herein, and are then considered a cohort having regulated expression in an identifiable temporal pattern. This seed pattern may include 2 or more previously identified genes. In some embodiments of the invention, the seed pattern may include 5 or 10 identified genes, and in some embodiments in may include 20, 30, 50, 100, 200, 500 or more previously identified genes. In one embodiment, the top 100 matches to the seed pattern are chosen as the transcripts with the strongest relevance for learning and memory function. In another embodiment, the top 50 matches to the seed pattern are chosen as the transcripts with the strongest relevance for learning and memory function. In yet the strongest relevance for learning and memory function.

In another embodiment of the invention, less than, all, or none of the transcripts demonstrating regulated expression following the learning and memory task or tasks have been previously identified to be involved in learning and memory processes. In this embodiment, some or all of the regulated genes following the learning and memory task have not been previously associated with learning and memory, but exhibit regulated expression in an identifiable temporal pattern.

In an embodiment of the invention, one or more of the transcripts exhibiting temporal regulation is a gene or combination of genes or gene fragments chosen from among any genes that have been previously identified to be associated with learning and memory, or chosen from the genes or gene fragments identified herein. For example, the genes or gene fragments can be chosen from translation initiation factors, heterogeneous nuclear ribonucleoproteins, RNA binding motif protein 9, ribosomal protein L5, translation elongation factors, synuclein alpha, tenascin R, smooth muscle alpha-actin, myelin-associated oligodendrocytic basic protein, low density lipoprotein receptor-related protein 3, dynamin I0like protein, ADP-ribosylation factor, kinesin family member, SNAP25, NSF, synaptoargnin IV, Atpla1, AtblbZ, calcium/calmodulin-dependent protein kinase II, MAPK1, MAPKK1, p38 MAPK, MAPK phosphatase, Ania-3, MAP2, protein kinase (cAMP-dependent catalytic and regulatory, beta), Ube2i, calnexin, cathepsinD, casolin-containing protein, Dnajb9, ATP citrate lyase, brain acyl-CoA hydrolase, sterol-coenzyme A denaturase 2, lipoprotein lipase, BDNF, IGF-2, IGF binding protein 2, fibroblast growth factor receptor 1, CD9 cell surface glycoprotein, Thy cell surface glycoprotein, G-protein coup led receptor, muscarinic receptor m2, chemokine receptor 5, GABA A, synaptogamins 7 and 8, syntaxin 2, 5, and 8, glutamate, serotonin transporters, inducible nitric oxide synthase, catenin, C-CAM2a, neurexin 1, connexin 43, contactin 1, chondroitin sulfate proteoglycan 3, myelin-associated glycoprotein, axonal glycoprotein, PSD-95NMDA-R2A, GluR6, GluR5-2, potassium channel subunits, BOD-L, homer 1a, syntaxin 1a, ERK2, PKB, mGLUR7, TRKB, VGF, CREM, gephyrin, N-cadherin, Rheb2, ARPP-21, EGR4, c-fos, c-jun, CRH, midkine, ttransthyretin, the bone morphogenetic antagonist, USAG-1, and eNNP2.

According to the invention, gene expression products include any products which have been or may be determined to be associated with, or be capable of modulating to either increase or decrease learning or memory, but are not limited to proteins, peptides, or transcription or translation.

Gene expression levels may be detected by methods known to those skilled in the art and may be obtained, for example using any apparatus that can measure gene expression levels which are widely known in the art. The nucleic acid molecule levels measured can be derived directly from the gene or, alternatively, from a corresponding regulatory gene. All forms of gene expression products can be measured, including, for example, spliced variants. Similarly, gene expression can be measured by assessing the level of protein or derivative thereof translated from mRNA. This may, however, also reflect posttranslational modifications and other forms of processing If the gene expression assessed is at the mRNA level, it can, for example, also be measured by in situ hybridization, Northern blot analysis, dot-blot hybridization analysis, microarray analysis, or by PCR. Such methods are described in detail, for example, in Ausubel et al., Current Protocols In Molecular Biology (New York: John Wiley & Sons) (1998); and Sambrook et al., Molecular Cloning: A Laboratory Manual, 2nd edition (New York: Cold Spring Harbor University Press (1989).

In another embodiment of the invention, gene expression levels can be obtained by contacting the sample of interest with a suitable microarray, and determining the extent of hybridization of the nucleic acid in the sample to the probes on the microarray. It is also contemplated by the invention that gene expression assessed by methods such as microarray can be validated by using quantitative real-time PCR analysis at the time points determined to be relevant from the temporal profile of gene expression observed. It will be apparent to those skilled in the art that any methodology that can be utilized for measuring gene expression will be suitable for use in the invention. Microarray analysis and PCR analysis can be carried out according to the Examples described herein, or by any of the available methods known in the art.

If the gene expression product is a protein or polypeptide, transcriptional regulation can be measured using techniques for protein detection and quantification that are known in the art. Antibodies, for example, can be generated which are specific to the protein using routine methods known in the art, which specifically bind to the protein of interest so that they can be detected and measured. Protein measurement can be carried out by any methods known in the art, including histochemistry, immunoblot analysis, in vitro binding studies, radioimmunoassay, and ELISAs.

and which are detected as a cohort of transcripts important in learning and memory processes are expressed sequence tags (ESTs). ESTs are short single-pass sequence reads from mRNA (cDNA). They may be of various lengths. For use in this invention, it is preferred, but not required, that they be of sufficient length to identify a unique expressed sequence. Typically, they are about 300-500 by in length. However, sequences as short as about 16 bases may be sufficient to identify a specific sequence. ESTs represent a snapshot of genes expressed in a given tissue and/or at a given developmental stage. They are tags (some coding, others not) of expression for a given cDNA library. There are now well over one million of these sequences in the publicly available database, and these sequences are believed to represent more than half of all human genes. The ESTs of, and for use with the invention, however, are not meant to be limited by what is available in public databases, and also may be novel ESTs that are generated.

The ESTs detected in the invention are useful for elucidating the genes and gene products responsible for regulating learning and memory processes, and therefore for understanding the mechanisms underlying disorders of learning and memory function. The ESTs detected according to the methods of this invention demonstrate specific regulation at time points expected to be involved in the learning and memory process, including memory consolidation. Such involvement may be to contribute, or be required, to allow for learning or memory maintenance or consolidation to occur. This is the case when less than all, or none of the transcripts are unknown, and even more so when the temporal patterns of the transcripts correspond to the temporal patterns of genes previously identified to be associated with learning and memory, during at least one of the time points assessed.

Once these ESTs are identified as being associated with learning or memory, at least one of the components of learning, acquisition, consolidation, or others, they are clearly useful for detecting or diagnosing learning and memory dysfunctions, as well as in modulating memory function by either enhancing or inhibiting gene expression or the gene expression product. Similarly, the ESTs for use with the invention are useful for development of new pharmaceutical agents that can be used to help those with diseases or disorders that influence learning and memory functions, as well as with enhancing learning and memory processes in people without dysfunction of learning and memory processes.

Some embodiments of the invention utilize learning and memory tasks in the methods to identify gene targets associated with learning and memory processes. “Task” as scenario which is capable of eliciting learning and memory processes. The invention is not limited by the means of eliciting and assessing learning and memory, and is meant to include anything which can cause learning and memory processes to take place, and which can be used to assess these processes.

The learning and memory tasks can be any such tasks that involve or elicit learning and memory processes. Depending on the specific type of learning and memory of interest, one may choose different tasks which yield data that are representative of different stages in the learning and memory process, different types of learning and memory, and which may represent the use or activation of different regions within the brain responsible for mediating those actions. One task contemplated by the present invention is the Morris water maze, which is one of the best-validated models of learning and memory. The test is a simple spatial learning task in which the animal is placed in tepid water, which may be opaque due to the addition of powdered milk. The animals learn the location of the platform relative to visual cues located within the maze and the testing room. Another important test contemplated for use in the invention is the passive avoidance test, including the one-trial, step-through, light-dark version of the passive avoidance paradigm as described previously (Fox et al., 1995, J Neurochem 65, 2796-2799). The odor-reward association paradigm is also contemplated for use with the invention. Yet another learning and memory test for use in the invention is the Y-maze test, which is based on visual discrimination. Other learning and memory tests which are contemplated as useful in the invention are eye blink conditioning, fear conditioning, and delayed match-to-position tasks. Also contemplated by the invention is the prepulse inhibition of startle response (PPI), which evaluates working memory. Any number of other tasks which elicit learning and memory processes known to the skilled practitioner will also be useful in the invention.

In some embodiments of the invention, the animals are humans. Accordingly, the tasks utilized in the invention would be appropriate for assessing learning and memory processes in humans, the appropriateness of which will be known to the skilled practitioner. Such tasks contemplated by the invention include accepted rating scales and standardized performance tests for cognitive function including, but not limited to CANTAB (Cambridge Neuropsychological Test Automated Battery), BEHAVE-AD (Behavioral Pathology in Alzheimer's Disease Rating Scale), Blessed Test, CERAD (The Consortium to Establish a Registry for Alzheimer's Disease), Clock Draw Test, Cornell Scale for Depression in Neuropsychiatric Inventory (NPI), The 7 Minute Screen, Children's Memory Scale (CMS), Continuous Recognition Memory Test (CMRT), Denman Neuropsychology Memory Scale, the Learning and Memory Battery (LAMB), Memory Assessment Clinic Self-Rating Scale (MAC-S), a Memory Assessment Scales (MAS), Randt Memory Test, Recognition Memory Test (RMT), and other clinical and neuropsychological tests.

The methods of detecting gene targets associated with learning and memory are generally carried out in animals other than humans. Once gene targets associated with learning and memory are identified according to the methods of the invention in animals other than humans, other aspects of the invention may be applied to other animals, including humans. In some embodiments, once the gene targets have been identified, it can be determined if those genes are present in humans, through, for example, sampling the blood or CSF. In some embodiments, it will be useful to determine if the detected gene targets, or their human equivalents are disrupted in humans, and if these disruptions are associated with alterations in learning and memory function, which can be assessed from a variety of tasks as described herein. If the genes, or targets, are present in humans, then one can modulate learning and memory function as described herein by modulating the activity of the identified genes or gene products, and confirming whether there has been a change in learning or memory function via various means of assessment. To assess a change in learning or memory function after modulation of an identified gene or gene product in animals other than humans, any of the tasks described herein which elicit learning and memory function may be used. To assess learning and memory function in humans, there are many tasks which may be utilized, known to those skilled in the art.

Systems for carrying out all of the methods described herein are also provided, and optionally comprise a computer system and related software. For example, as relates to the foregoing method, the system may comprise groups of mice that have been administered a plurality of learning, memory, and/or memory consolidation tasks and a computer system comprising software, said computer system configured to (a) detect a cohort of transcripts of which expression is regulated in an identifiable temporal pattern following at least two of the learning tasks; (b) compare the identifiable temporal patterns of regulated expression from the cohorts detected in (a); and (c) detect a cohort of transcripts from (b) wherein said transcripts are temporally regulated in tissue from animals administered a different learning, memory, and/or memory consolidation task, and which cohort comprises transcripts present least two of the learning tasks. Optionally, the computer system may output a result from (c) which is indicative of gene targets associated with learning, memory, and/or memory consolidation.

The targets obtained according to the methods of the invention, described above, can be used to modulate learning and memory function by being either upregulated or downregulated, or by increasing of decreasing their products. The invention is also useful for screening drug candidates which modulate the activity of the targets obtained according to the methods of the invention, and are therefore useful to modulate learning and memory. The invention is also useful for identifying possible mechanisms of learning or memory deficiency. It can be determined whether targets identified according to the methods of the invention are involved in such conditions.

In yet another embodiment, the invention provides methods of modulating memory function by enhancing the gene expression or the gene expression product of at least one of the nucleic acid sequence identified to be associated with learning and memory according to the methods of this invention. Accordingly, through the temporal analysis of expression patterns, gene targets are identified that may serve as targets for modulating memory, either by increasing or decreasing the function of the gene of interest, or a product of the gene of interest. In certain embodiments, it may be desirable to increase learning and memory function, whereas in other embodiments, it may be desirable to decrease the function of the gene of interest of a product of the gene of interest. Included among the gene targets for modulating memory function are nucleic acid sequences selected from the group consisting of SEQ ID NO:1 through SEQ ID NO:11.

In another embodiment, the invention provides methods of modulating memory function by downregulating or reducing the gene expression or the gene expression product of at least one of the nucleic acid sequence identified to be associated with learning and memory according to the methods of this invention. Included among the targets for decreasing gene expression are at least one of the nucleic acid sequences of SEQ ID NO:1 through SEQ ID NO:11. Downregulating or inhibiting learning or memory is useful where it is desirable for an individual not to remember or recall an experience or event, as described below.

limited to proteins, peptides, or nucleic acid molecules (e.g., mRNA, tRNA, rRNA, or cRNA) that are involved in transcription or translation.

In one embodiment of the invention, gene expression products can refer to epigenetic changes such as DNA methylation and chromatin remodeling. Chromatin remodeling is initiated by the posttranslational modification of the amino acids that make up histone proteins, or by the addition of methyl groups to the DNA at CpG sites to convert cytosine to 5-methylcytosine. Any epigenetic modulation known to one of skill in the art is contemplated by the invention.

Also contemplated by the invention are methods of diagnosing a learning or memory disorder or dysfunction in an animal. In one embodiment of this invention, the animal is a human. Such a disorder can be diagnosed by evaluating the expression of at least one nucleic acid sequence or its product identified to be associated with learning and memory according to the methods of the invention, either at a single time point, or over time. One can determine how such a nucleic acid sequence differs in its expression at one or more time points following any activity known to elicit learning and memory function, or its expression may be measured at baseline conditions. A learning and memory disorder may also be assessed by modulating the activity of at least one nucleic acid sequence or its product as described above, to assess if learning and memory function is altered through the modulation. Included among the gene targets for diagnosing a learning and memory disorder are nucleic acid sequences detected by the methods according to the invention, and in particular, may include, but not be limited to, those selected from the group consisting of SEQ ID NO:1 through SEQ ID NO:11.

Impairments and abnormalities in learning and memory processes can occur in a number of conditions or diseases. Non limiting examples of such conditions and diseases that may be treated by the methods and compositions of this invention include, but are not limited to age-related memory loss, dementia, senile dementia, Mild Cognitive Impairment, Alzheimer's disease, senile dementia of the Alzheimer's type, Multiple Sclerosis, brain injury, amnesia, neuronal toxicity, brain aneurysm, stroke, schizophrenia, epilepsy, chronic fatigue syndrome, fibromyalgia syndrome, chemotherapy (e.g., cancer chemotherapy), traumatic brain injury, Parkinson's disease, and post-traumatic stress disorder (PTSD). treatment of impairments and abnormalities in learning and memory processes through modulating the gene expression or gene expression product of at least one nucleic acid sequence identified according to the methods of this invention. In certain embodiments, the nucleic acid is selected from the group consisting of SEQ ID NO:1 through SEQ ID NO:11. The aim is to enhance learning and memory function in individuals with reductions in normal learning and memory processes, for example, such as those having any of the conditions stated above, an in particular, those with Alzheimer's disease, schizophrenia, and stroke. In those with memory abnormalities such as PTSD, in which memories of unpleasant experiences are actually enhanced, the invention contemplates downregulating or reducing relevant gene expression or gene expression products to reduce or alleviate symptoms.

Modulating the nucleic acid sequences and gene products identified through this invention may be accomplished by increasing or decreasing their activity depending on the desired result. This will vary, depending on whether the goal is to enhance or reduce learning or memory. For the conditions mentioned above, where enhancement is desired, one may seek to enhance those sequences that demonstrate an increase in expression over time in the temporal patterns observed during or following a learning and memory task, depending on the nature of the task administered, and depending on the specific time points at which increased expression is observed. One may also seek to reduce those sequences that decrease during or following a learning and memory task, with the goal of enhancing learning and memory depending on the nature of the task administered, and depending on the specific time points at which decreased expression is observed.

In one embodiment, in order to modulate the nucleic acid sequences and gene products identified through this invention, it may be desirable to enhance the gene expression or gene expression product that is observed increasing expression 2 hours, 12 hours, or 24 hours after a learning and memory task. More specifically, one may enhance the gene expression or gene expression product after training on the Morris water maze. In another embodiment, it may be desirable to enhance the gene expression or gene expression product that is observed decreasing expression 1 hour after a learning and memory task, and, more specifically, after training on a passive avoidance task. In yet other embodiments, it may be desirable to enhance or inhibit the gene expression or gene expression products identified according to the methods of the invention by intervening at different times either prior to, during, or after a learning and memory task, such as, for example, at 0, 1, 2, 12, or 24 hours following the task. Modulating the nucleic acid sequences and gene products identified through the methods of this invention may be for the purpose of treating a learning and memory disorder, for enhancing learning and memory, or in other embodiments, for identifying compounds useful for modulating learning and memory function.

In certain situations it may be desirable to reduce the learning or memory of an individual, particularly following exposure to a traumatic experience. Such an experience may be following witnessing a crime, post traumatic stress syndrome, surgical procedures, or other experiences the memory of which may cause harm to the individual. In humans where consolidations may take up to ten (10) years or more to be complete, the experience may be reconsolidated so that it is susceptible to the modulations contemplated by this invention. Where reductions in learning and memory are desired, one may seek to reduce those sequences that demonstrate an increase in expression over time in the temporal patterns observed during or following a learning and memory task depending on the nature of the task administered. One may also seek to enhance those sequences that decrease during or following a learning and memory task, with the goal of reducing learning and memory depending on the nature of the task administered.

Any means of enhancing gene expression, or the gene expression product in an individual that is known by one of skill in the art may be used to modulate gene expression or a gene expression product as contemplated for methods of treatment in the invention. For example, pharmaceutical compositions which are agonists for the gene product of interest may be used to enhance learning and memory processes.

Any means of reducing gene expression, or the gene expression product in an individual that is known by one of skill in the art may be used to reduce gene expression or a gene expression product as contemplated for methods of treatment in the invention. Non-limiting examples for use in reducing gene expression or gene expression products according to the invention include RNA interference, antisense RNA, antibodies, and pharmaceutical compositions that antagonize the activity of the gene product of interest.

In certain embodiments, antisense is used to decrease expression of a gene. Antisense is used in reference to RNA sequences that are complementary to a specific RNA sequence (e.g., mRNA). Included within antisense are antisense RNA (“asRNA”) molecules involved in gene regulation by bacteria. Antisense RNA may be produced by any method, promoter that permits the synthesis of a coding strand. Once introduced, this transcribed strand combines with natural mRNA to form duplexes. These duplexes then block either the further transcription of the mRNA or its translation. Regions of a nucleic acid sequences that are accessible to antisense molecules can be determined using available computer analysis methods.

In certain embodiments, an RNA interference (RNAi) molecule is used to decrease expression of a gene. RNA interference (RNAi) is defined as the ability of double-stranded RNA (dsRNA) to suppress the expression of a gene corresponding to its own sequence. RNAi is also called post-transcriptional gene silencing or PTGS. Since the only RNA molecules normally found in the cytoplasm of a cell are molecules of single-stranded mRNA, the cell has enzymes that recognize and cut dsRNA into fragments containing 21-25 base pairs (approximately two turns of a double helix). The antisense strand of the fragment separates enough from the sense strand so that it hybridizes with the complementary sense sequence on a molecule of endogenous cellular mRNA. This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

Double-stranded RNA can be used to interfere with gene expression in mammals (Wianny & Zemicka-Goetz, 2000, Nature Cell Biology 2: 70-75; incorporated herein by reference in its entirety). dsRNA is used as inhibitory RNA or RNAi of the function of the gene of interest to produce a phenotype that is the same as that of a null mutant of the gene of interest (Wianny & Zernicka-Goetz, 2000, Nature Cell Biology).

In another embodiment, as method is provided which comprises screening compounds for their ability to increase or decrease the activity of one or more of the targets identified according to the methods of the invention. Accordingly, an assay is provided which comprises administering a test compound to a group of animals, and in some embodiments, a placebo to a groups of animals, and detecting changes in the expression profile following one or more learning and memory tasks as described above. A test compound is any compound of interest, wherein one is interested in determining the compound's involvement in learning and memory. Such compounds may then be identified as drug candidates for modulating learning, memory, or memory consolidation. Compounds can also be identified by comparing the gene expression profiles to those of the gene expression profiles of those selected from the group consisting of SEQ ID NO:1 through SEQ ID NO:11. In addition to nucleic acids, such as siRNA sequences described above, other compounds that bind to the gene products either competitively or non-competitively, or otherwise affect their activity may be useful as drug candidates for modulating learning, memory, or memory consolidation.

Also contemplated by the invention are isolated nucleic acid molecules identified according to the methods of this invention. In one embodiment, this invention comprises a nucleic acid sequence selected from the group consisting of SEQ ID NO:1 through SEQ ID NO:11.

Pharmaceutical compositions comprising the genes or gene fragments derived according to the methods of the invention, including the nucleic acids from SEQ ID NO:1 through SEQ ID NO:11 are also contemplated by the invention.

The pharmaceutical compositions of the invention can be formulated as natural or salt forms. Pharmaceutically acceptable non-toxic salts include the base addition salts (formed with free carboxyl or other anionic groups) which may be derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts may also be formed as acid addition salts with any free cationic groups and will generally be formed with inorganic acids such as, for example, hydrochloric, sulfuric, or phosphoric acids, or organic acids such as acetic, p-toluenesulfonic, methanesulfonic acid, oxalic, tartaric, mandelic, and the like. Salts of the invention include amine salts formed by the protonation of an amino group with inorganic acids such as hydrochloric acid, hydrobromic acid, hydroiodic acid, sulfuric acid, phosphoric acid, and the like. Salts of the invention also include amine salts formed by the protonation of an amino group with suitable organic acids, such as p-toluenesulfonic acid, acetic acid, and the like. Additional excipients which are contemplated for use in the practice of the present invention are those available to those of ordinary skill in the art, for example, those found in the United States Pharmacopeia Vol. XXII and National Formulary Vol. XVII, U.S. Pharmacopcia Convention, Inc., Rockville, Md. (1989), the relevant contents of which are incorporated herein by reference.

in part by the specific composition administered, as well as by the particular method used to administer the composition. Accordingly, there are a wide variety of suitable formulations of pharmaceutical compositions of the present invention (see, e.g., Remington's Pharmaceutical Sciences, 20th ed., 2003).

The term “pharmaceutically acceptable” can mean approved by a regulatory agency of the Federal or a state government or listed in the U.S. Pharmacopeia or other generally recognized pharmacopeia for use in animals, and for use in humans. The term “carrier” can mean a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions are sterile and in suitable form for administration to a subject, preferably an animal subject, more preferably a mammalian subject, and most preferably a human subject.

In another embodiment, the composition can be delivered in a vesicle, in particular a liposome (see Langer, Science 249:1527-1533 (1990); Treat et al., in Liposomes New York, pp. 353-365 (1989); Lopez-Berestein, pp. 317-327).

In yet another embodiment of the invention, the composition can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; International Publication No. WO 99/15154; and International Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the lungs, thus requiring only a fraction of the systemic dose (see, e.g., Goodson, in Medical Applications of Controlled Release, supra, vol. 2, pp. 115-138 (1984)).

Controlled release systems are discussed in Langer (1990, Science 249:1527-1533). Any technique known to one of skill in the art can be used to produce sustained release formulations comprising one or more antibodies of the invention or fragments thereof. See, e.g., U.S. Pat. No. 4,526,938, International publication No. WO 91/05548, International publication No. WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In some embodiments of the invention, in which the composition is one or more nucleic acid molecules obtained according to the methods of the invention, the nucleic acid or nucleic acids can be administered in vivo by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

Pharmaceutical compositions of the invention are formulated to be compatible with their intended route of administration. Examples of suitable routes of administration include, but are not limited to, parenteral (e.g., intravenous, intramuscular, intradermal, intra-tumoral, intra-synovial, and subcutaneous), oral (e.g., inhalation), intranasal, transdermal (topical), transmucosal, intra-tumoral, intra-synovial, vaginal, and rectal administration. In a specific embodiment, the composition is formulated in accordance with routine procedures as a pharmaceutical composition adapted for intravenous, subcutaneous, intramuscular, oral, intra-tummoral, intra synnovial, intranasal or topical administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

If the compositions of the invention are to be administered orally, the compositions can be formulated orally in the form of, e.g., gum, tablets, capsules, cachets, gelcaps, solutions, suspensions and the like. Tablets or capsules can be prepared by conventional means with pharmaceutically acceptable excipients such as binding agents (e.g., pregelatinised maize starch, polyvinylpyrrolidone or hydroxypropyl methylcellulose); fillers magnesium stearate, talc or silica); disintegrants (e.g., potato starch or sodium starch glycolate); or wetting agents (e.g., sodium lauryl sulphate). The tablets may be coated by methods well-known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated for slow release, controlled release or sustained release of a prophylactic or therapeutic agent(s).

If the compositions of the invention are to be administered intranasally, the compositions can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compositions of the invention may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

compositions can be formulated in the form of, e.g., an, ointment, cream, transdermal patch, lotion, gel, oral gel, spray, aerosol, solution, emulsion, or other form well-known to one of skill in the art. See, e.g., Remington's Pharmaceutical Sciences and Introduction to Pharmaceutical Dosage Forms, 4.sup.th ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well-known in the art.

The compositions of the invention may also be formulated in rectal compositions such as suppositories or retention enemas, e.g., containing conventional suppository bases such as cocoa butter or other glycerides.

In addition to the formulations described above, the compositions of the invention may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

separately or mixed together in unit dosage form, for example, as a dry lyophilized powder or water free concentrate in a hermetically sealed container such as an ampoule or sachette indicating the quantity of active agent. If the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### EXAMPLES

It is understood that the following examples and embodiments described herein are for illustrative purposes only and that various modifications or changes in light thereof will be suggestive to persons skilled in the art and are to be included within the spirit and purview of this application and the scope of the appended claims. All publications, patents, and patent applications cited herein are hereby incorporated by reference in their entirety for all purposes.

Unless otherwise specified, in the examples provided below, animals were maintained as follows.

Animal Maintenance

Postnatal day 80 male Wistar rats (300-350 g) were obtained from the Biomedical Facility at University College Dublin and were group housed on a 12:12 light/dark cycle, with ad libitum access to food and water. The animals were introduced into the experimental rooms 5 days prior to commencement of training On the 2 days prior to training the animals were handled, their weights monitored and spontaneous behavior assessed in an open field apparatus (620 mm long, 620 mm wide, 150 mm high). The floor of the open field was ruled into a series of squares (77×77 mm), the animal was placed in the centre and the number of lines crossed in a 5-minute period counted. Other behaviors assessed included rearing, grooming, piloerection, defecation and posture. All observations were carried out in the quiet room under low-level red illumination between 08:00 and 12:00 to minimize circadian influence. Naïve littermates were treated precisely as their trained counterparts except they were not exposed to the training environment prior to sacrifice.

### mRNA Expression Profiles Following Learning Tasks

Materials and Methods

Water Maze Training

The spatial learning task has been described in detail previously (Murphy et al., 1996). The water maze apparatus consisted of a large circular pool (1 m diameter, 80 cm high, temperature 26±1° C.) with a platform (11 cm diameter) submerged 1.5 cm below the water surface. Both the pool and the platform were constructed of black polyvinyl plastic and offered no intra-maze cues to guide escape behavior. The experimental room contained several extra-maze visual cues. During training the platform was hidden in the same quadrant 30 cm from the edge of the maze. Each trial started with the rat facing the wall of the maze at one of three locations. The time taken by the rat to find the hidden platform within a 60 sec period was recorded. On the first trial, rats failing to find the platform within the 60 sec period were placed on it for 10 sec. Times to the platform were measured over 5 trials in the training session with an inter-trial interval of 300 sec. To control for stress and other non-learning associated factors during water maze training each trained animal was paired with a corresponding passive control animal that was allowed to swim in the maze for a time matching its trained counterpart for each trial but in the absence of a platform.

Passive Avoidance Training

On the day of training, spontaneous behaviors were assessed prior to passive avoidance training The one-trial, step-through, light-dark version of the passive avoidance paradigm was employed as we have described previously (Fox et al. 1995). Briefly, the smaller illuminated compartment was separated from the larger dark compartment by a shutter that contained a small entrance. The floor of the training apparatus consisted of a grid of stainless steel bars that would deliver a scrambled shock (0•75 mA every 0•5 ms) for 5 seconds when the animal entered the dark chamber. Passive controls were exposed to the avoidance apparatus for a time matched to trained counterparts but never received a foot-shock.

Sample Collection

In order to determine the sequential expression of mRNA in the rat dentate gyrus during memory consolidation, animals were sacrificed at discrete time points 0, 0.5, 1, were killed by cervical dislocation, the dentate gyms rapidly dissected and snap frozen. All experimental procedures were approved by the Animal Research Ethics Committee of the Biomedical Facility at University College, Dublin, and were carried out by individuals who held the appropriate licence issued by the Minister for Health and Children.

Microarray and Real-Time Sample Preparation

Total RNA was extracted from each dentate gyms by homogenization in TRIzol reagent (Invitrogen, Carlsbad, Calif., USA) and following the TRIzol protocol. The resulting RNA samples were purified using an RNeasy mini kit (Qiagen, UK). RNA concentration was determined spectrophotmetrically, and RNA integrity was confirmed by agarose gel electrophoresis. Double-stranded cDNA was synthesised from 10 μg total RNA (Superscript System, Invitrogen, Carlsbad, Calif., USA). Briefly, the RNA was mixed with 100 pmol oligonucleotide GGC CAT GGAAT TGTAATAC GAC T CAC TATAGGGAGGC GG (dT)24 in 20 μl water, annealed at 70° C. for 10 min, and quick-chilled. Buffer, dithiothereitol, and dNTP mix were than added and incubated at 37° C. for 2 min. Second-strand synthesis was performed by adding reaction buffer, dNTPs (200M), DNA ligase (10 U), DNA polymerase (40 U), ribonuclease H (2 U), and water (to a final volume of 150 μl), and the reaction was incubated for 2 h at 16° C. This was followed by addition of 10 U T4 DNA polymerase and incubation at 16° C. for 5 min. The cDNA was purified by phenol/chloroform extraction, precipitated, and transcribed in vitro using T7 RNA polymerase. Biotinylated cRNA was generated using the BioArray HighYield RNA Transcription Kit (Enzo Diagnostics, Inc., Farmingdale, N.Y.). The cRNA was purified by RNeasy minispin columns and fragmented by incubation in 40 mM Tris (pH 8.1), 100 mM potassium acetate, and 30 mM magnesium acetate buffer at 94° C. for 35 min.

Microarray Analysis

Fragmented cRNA for each sample (n=6×8 time points×2 (trained & passive control)=96 samples per training paradigm) was hybridised to the Affymetrix rat genome RG230.02 chip using the protocol outlined in the GeneChip Expression Analysis Technical Manual (Affymetrix Inc., Santa Clara, Calif., USA). Hybridised chips were washed and stained using Affymetrix Fluidics Station 400 and EukGE-WS 1 Standard Format as recommended by the manufacturer. The staining was performed using streptacidin-phycoerythrin conjugate (SAPE; Molecular Probes, Eugene, Oreg., USA), followed by biotinylated antibody against scanned using a Hewlett-Packard GeneArray Scanner and analysed using Affymetrix MAS5.0 software. Hybridization intensities were normalised using a method featuring a pool of 11 biotin-labelled cRNA control transcripts, derived by in vitro transcription of 11 cloned Bacillus subtilis genes, which were spiked into each hybridization experiment. This normalization method has been described in detail previously (Hill et al., 2001). The 5′/3′ ratio for glyceraldehydes-3-phosphate dehydrogenase (GAPDH) and for beta-actin ranged from 0.8 to 1.1.

DNA microarrays were used to study the mRNA expression profiles of rat dentate gyms at increasing times following either water maze spatial learning or avoidance conditioning. Approximately 5500 and 2500 genes were transcriptionally regulated across the 24 h post-training period following spatial learning and avoidance conditioning, respectively. When these gene lists were compared, a substantial cohort consisting of 1500 transcripts was identified whose expression levels were regulated following both learning tasks. Collectively, these genes can be considered a core transcriptional program for memory consolidation, deployed independent of the nature of the learning task.

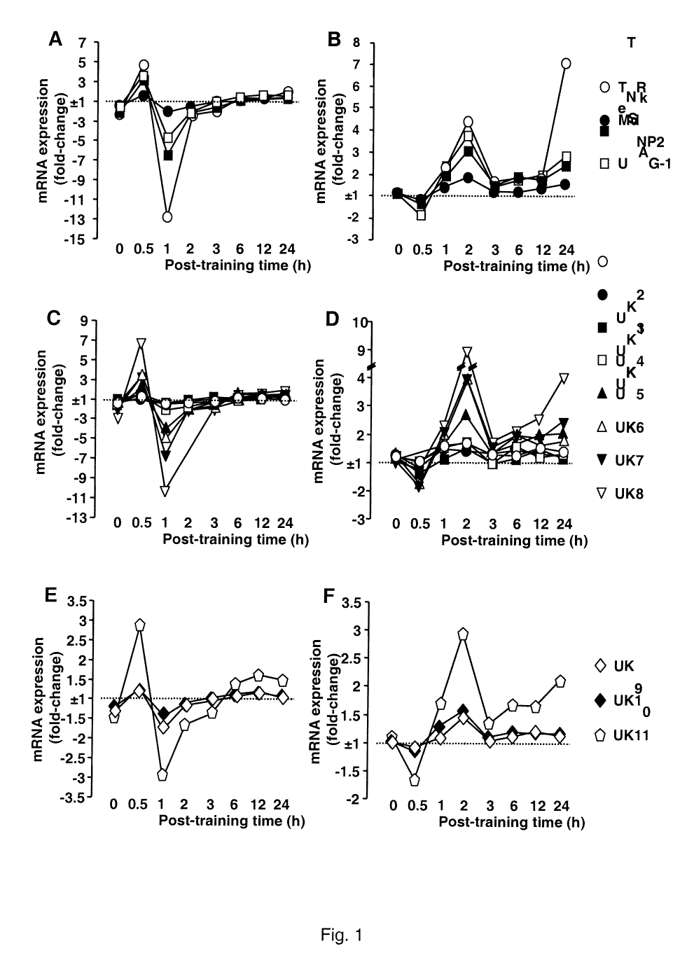

Analysis of the known genes in this core group revealed several transcripts of interest with respect to a potential role in memory-associated synaptic plasticity such as the novel neurotrophin, midkine (Mdk), the carrier molecule, Transthyretin (Ttr), USAG-1, and eNNP2. These transcripts shared a characteristic temporal pattern of regulation following water maze learning that of learning-specific increases in expression at the 2 and 24 h post-training times (FIG. 1). Subsequent study of this gene cluster revealed that this same cohort co-regulated following passive avoidance learning. The temporal pattern, up at 30 minutes, down at 1 h returning to basal levels thereafter, was distinct from that seen in the water maze.

Clusters of unknown genes were identified that shared these temporal patterns of regulation with the known genes in both tasks (FIG. 1). This was determined by using the average temporal pattern of the known genes as a seed pattern and ranking all other regulated genes based on their similarity to this pattern. The cluster claimed rank in the top 50 matches for both water maze and passive avoidance learning. The potential of the unknown genes as targets for regulating cognitive function is underpinned by the validation of the co-regulating known genes at message, protein and functional levels. Information about the unknown gene transcripts is provided in Table 1.

| Code | Affy qualifier | Gene Name |
|---|---|---|
| UK1 | 1391211_at | Atpase, class VI, type 11C |
| | | (predicted) |
| UK2 | 1374172_at | Unknown transcript |
| UK3 | 1385528_at | Similar to phosphorylase |
| | | kinase alpha 2 |
| UK4 | 1376980_at | Unknown transcript |
| UK5 | 1375933_at | Claudin 2 |
| UK6 | 1378365_at | Solute carrier family 4, |
| | | sodium bicarbonate |
| | | cotransporter, member 5 |
| UK7 | 1392948_at | Chloride intracellular channel 6 |
| UK8 | 1397185_at | Klotho |
| UK9 | 1374094_at | Similar to 1500031K13Rik |
| | | protein |
| UK10 | 1373790_at | Similar to Aph1a-pending |
| | | protein |
| UK11 | 1377434_at | Membrane-type frizzled- |
| | | related protein |

Genes and gene fragments identified as changing significantly in animals at time 0 following water maze training relative to controls are provided in Table 2 below.

TABLE 2

| Fragment Name | Gene Name | Gene Symbol |
|---|---|---|
| 1367999_at | (aldehyde dehydrogenase 2, hypothetical gene supported by | (Aldh2, na) |
| | NM_032416) | |
| 1381509_at | (ovarian carcinoma antigen CA125, similar to Nbr1) | (Ca125, na) |
| 1387436_at | (CDC10 (cell division cycle 10, S. cerevisiae, homolog), similar | (Cdc10, na) |
| | to cell division cycle 10 homolog) | |
| 1367732_at | (guanine nucleotide binding protein, beta 1, similar to guanine | (Gnb1, na) |
| | nucleotide-binding protein, beta-1 subunit) | |
| 1371102_x_at | (hemoglobin beta chain complex, similar to Hemoglobin beta | (Hbb, na) |
| | chain, minor-form) | |
| 1371245_a_at | (hemoglobin beta chain complex, similar to Hemoglobin beta | (Hbb, na) |
| | chain, minor-form) | |
| 1390990_at | (protein phosphatase 1, catalytic subunit, beta isoform, protein | (LOC316717, Ppp1cb) |
| | serine-threonine phosphatase catalytic subunit PP-1c) | |
| 1389734_x_at | (MHC class I RT1.O type 149 processed pseudogene, RT1 | (LOC360231, RT1-Aw2, RT1-T24-1) |
| | class Ib, locus Aw2, histocompatibility 2, T region locus 24) | |
| 1370948_a_at | (myristoylated alanine rich protein kinase C substrate, similar to | (Marcks, na) |
| | Myristoylated alanine-rich C-kinase substrate (MARCKS)) | |
| 1370949_at | (myristoylated alanine rich protein kinase C substrate, similar to | (Marcks, na) |
| | Myristoylated alanine-rich C-kinase substrate (MARCKS)) | |
| 1371776_at | (hypothetical gene supported by NM_013005, | (Pik3r1, na) |
| | phosphatidylinositol 3-kinase, regulatory subunit, polypeptide | |
| | 1) | |
| 1386992_at | (prostaglandin E receptor 1, protein kinase N1) | (Pkn1, Ptger1) |
| 1375378_at | (homolog of mouse quaking QKI (KH domain RNA binding | (Qki, na) |
| | protein), similar to QKI isoform 7B) | |
| 1391078_at | (replication factor C, similar to replication factor C) | (Recc1, na) |
| 1370428_x_at | (RT1 class I, A3, RT1 class Ib, locus Aw2, RT1 class Ib, locus | (RT1-A3, RT1-Aw2, RT1-Cl) |
| | Cl) | |
| 1386909_a_at | (similar to Voltage-dependent anion-selective channel protein 1 | (Vdac1, na) |
| | (VDAC-1) (mVDAC1) (mVDAC5) (Outer mitochondrial | |
| | membrane protein porin 1) (Plasmalemmal porin), voltage- | |
| | dependent anion channel 1) | |
| 1367706_at | (similar to Voltage-dependent anion-selective channel protein 1 | (Vdac1, na) |
| | (VDAC-1) (mVDAC1) (mVDAC5) (Outer mitochondrial | |
| | membrane protein porin 1) (Plasmalemmal porin), voltage- | |
| | dependent anion channel 1) | |
| 1383052_a_at | (similar to zinc finger protein 91 isoform 1, zinc finger protein | (Zfp91, na) |
| | 91) | |
| 1370857_at | smooth muscle alpha-actin | Acta2 |
| 1382981_at | Abelson helper integration site 1 | Ahi1 |
| 1369326_at | A kinase (PRKA) anchor protein 6 | Akap6 |
| 1367982_at | aminolevulinic acid synthase 1 | Alas1 |
| 1369063_at | acidic (leucine-rich) nuclear phosphoprotein 32 family, member A | Anp32a |
| 1369198_at | apoptotic protease activating factor 1 | Apaf1 |
| 1378372_at | arginase 2 | Arg2 |
| 1370324_at | ATPase, Cu++ transporting, beta polypeptide | Atp7b |
| 1386991_a_at | bcl-2 associated death agonist | Bad |
| 1387629_at | B-box and SPRY domain containing | Bspry |
| 1368585_at | cocaine and amphetamine regulated transcript | Cart |
| 1370832_at | small inducible cytokine A4 | Ccl4 |
| 1381930_at | chaperonin containing TCP1, subunit 5 (epsilon) | Cct5 |
| 1375719_s_at | cadherin 13 | Cdh13 |
| 1368887_at | cadherin 22 | Cdh22 |
| 1387674_at | cannabinoid receptor 1 (brain) | Cnr1 |
| 1370959_at | collagen, type III, alpha 1 | Col3a1 |
| 1368584_a_at | complexin 2 | Cplx2 |
| 1370026_at | crystallin, alpha B | Cryab |
| 1384339_s_at | casein kinase II, alpha 1 polypeptide | Csnk2a1 |
| 1379480_at | diacylglycerol kinase, iota | Dgki |
| 1398276_at | discs, large (Drosophila) homolog 2 (chapsyn-110) | Dlg2 |
| 1382778_at | dual specificity phosphatase 6 | Dusp6 |
| 1388358_at | electron-transfer-flavoprotein, beta polypeptide | Etfb |
| 1374320_at | coagulation factor 5 | F5 |
| 1368829_at | fibrillin-1 | Fbn1 |
| 1368241_a_at | flotillin 1 | Flot1 |
| 1387685_at | frequenin homolog (Drosophila) | Freq |
| 1369371_a_at | gamma-aminobutyric acid (GABA) B receptor, 1 | Gabbr1 |
| 1392818_at | growth arrest specific 5 | Gas5 |
| 1371153_a_at | glycine receptor, alpha 2 subunit | Glra2 |
| 1387670_at | glycerol-3-phosphate dehydrogenase 2 | Gpd2 |
| 1370849_at | hyaluronan and proteoglycan link protein 2 | Hapln2 |
| 1370103_at | hyperpolarization-activated, cyclic nucleotide-gated potassium | Hcn1 |
| | channel 1 (HCN1) | |
| 1368870_at | Inhibitor of DNA binding 2, dominant negative helix-loop-helix | Id2 |
| | protein | |
| 1369626_at | insulin degrading enzyme | Ide |
| 1393347_at | integrin alpha L | Itgal |
| 1387698_at | potassium inwardly rectifying channel, subfamily J, member 11 | Kcnj11 |
| 1390891_at | kinesin-like 1 | Kif11 |
| 1382787_at | kinesin family member 5A | Kif5a |
| 1389250_at | lunatic fringe gene homolog (Drosophila) | Lfng |
| 1387946_at | lectin, galactoside-binding, soluble, 3 binding protein | Lgals3bp |
| 1368368_a_at | liver-specific bHLH-Zip transcription factor 7 | Lisch7 |
| 1375726_at | LIM domain only protein 7 | LMO7 |
| 1389876_at | CaM-kinase II inhibitor alpha | LOC287005 |
| 1386966_a_at | protein kinase C-binding protein Beta15 | LOC60383 |
| 1368679_a_at | lyn protein non-receptor kinase | Lyn |
| 1387646_a_at | Max protein | Max |
| 1385101_a_at | Unknown (protein for MGC: 73017) | MGC73017 |
| 1376446_at | similar to RIKEN cDNA D430028G21 | MGC93707 |
| 1371919_at | similar to RP2 protein, testosterone-regulated - ricefield mouse | MGC94056 |
| | (Mus caroli) | |
| 1389638_at | similar to 4632419K20Rik protein | MGC94288 |
| 1367860_a_at | matrix metalloproteinase 14 (membrane-inserted) | Mmp14 |
| 1376058_at | mitochondrial tumor suppressor 1 | Mtus1 |
| 1368310_at | myogenin | Myog |
| 1376263_at | similar to 1810034B16Rik protein | na |
| 1393407_at | similar to F-box protein FWD2 | na |
| 1382024_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1395409_at | similar to protein phosphatase 2A, regulatory subunit B (PR 53) | na |
| 1393881_at | similar to N-terminal aceyltransferase 1 | na |
| 1384388_at | similar to gp25L2 protein | na |
| 1385546_at | similar to PC326 protein | na |
| 1379101_at | similar to DEAD/H box polypeptide 36 protein | na |
| 1389085_at | hypothetical LOC287534 | na |
| 1381926_at | similar to chromosome 20 open reading frame 169 | na |
| 1392643_at | similar to RAB5B, member RAS oncogene family | na |
| 1377940_at | hypothetical LOC287534 | na |
| 1389261_at | similar to hypothetical protein MGC23280 | na |
| 1391578_at | similar to modulator of estrogen induced transcription | na |
| 1384402_a_at | similar to PC326 protein | na |
| 1389572_at | similar to NADP-dependent malic enzyme, mitochondrial | na |
| | precursor (NADP-ME) (Malic enzyme 3) | |
| 1382489_at | similar to WD repeat domain 11 protein | na |
| 1388704_at | similar to CUG triplet repeat RNA-binding protein 1 (CUG- | na |
| | BP1) (RNA-binding protein BRUNOL-2) (Deadenylation | |
| | factor CUG-BP) (Deadenylation factor EDEN-BP) (Brain | |
| | protein F41) | |
| 1385423_at | similar to inner membrane protein, mitochondrial (mitofilin); | na |
| | motor protein | |
| 1385282_at | similar to hypothetical protein A430110N23 | na |
| 1392051_at | similar to hypothetical protein FLJ14681 | na |
| 1385109_at | similar to RIKEN cDNA 4930553M18 | na |
| 1398581_at | similar to grey lethal osteopetrosis; grey-lethal; grey lethal | na |
| | osteroperosis | |
| 1392514_at | similar to RIKEN cDNA 2810470K21 gene | na |
| 1393614_at | similar to amyloid beta (A4) precursor protein-binding, family | na |
| | A, member 2 binding protein isoform 1; X11L-binding protein | |
| | 51; synaptotagmin interacting protein STIP3; amyloid beta (A4) | |
| | precursor protein-binding, family A, member 2; synaptotagmini . . . | |
| 1379765_at | (similar to nemo-like kinase, similar to nemo-like kinase; likely | na |
| | ortholog of mouse nemo like kinase) | |
| 1396629_at | similar to ORM1-like 3 | na |
| 1389621_at | (similar to 1300019H17Rik protein, similar to FIP1-like 1; | na |
| | rearranged in hypereosinophilia) | |
| 1374484_at | similar to RIKEN cDNA 2610033C09 | na |
| 1384937_at | similar to RB-associated KRAB repressor | na |
| 1379250_at | similar to RIKEN cDNA A530057M15 gene | na |
| 1377571_at | similar to zinc finger protein | na |
| 1384619_at | hypothetical LOC288012 | na |
| 1395734_at | similar to palmdelphin | na |
| 1375749_at | similar to 2810407P21Rik protein | na |
| 1375528_at | similar to intracellular membrane-associated calcium- | na |
| | independent phospholipase A2 gamma | |
| 1377182_at | similar to RIKEN cDNA 4930471O16 | na |
| 1383106_at | similar to Cul2 protein | na |
| 1393810_at | similar to RIKEN cDNA 1810073M12 | na |
| 1382269_at | similar to cyclin M2; ancient conserved domain protein 2 | na |
| 1379955_at | similar to KIAA1337 protein | na |
| 1379249_at | similar to acetyl CoA transferase-like | na |
| 1393037_at | similar to RIKEN cDNA A930014C21 | na |
| 1393706_at | similar to six transmembrane epithelial antigen of the prostate | na |
| 1379868_at | similar to RIKEN cDNA 4732463H20 | na |
| 1374301_at | similar to ldlBp | na |
| 1375176_at | similar to Sperm 1 POU-domain transcription factor (SPRM-1) | na |
| 1384331_at | similar to RIKEN cDNA 1700127B04 | na |
| 1376037_at | similar to RIKEN cDNA D030041N15 | na |
| 1390357_at | similar to zinc finger protein 35 | na |
| 1372033_at | similar to RIKEN cDNA 0610042E07 | na |
| 1378921_at | similar to ETO/MTG8-related protein ETO-2 | na |
| 1381323_at | similar to retinoblastoma binding protein 5 | na |
| 1380688_at | hypothetical LOC293181 | na |
| 1371620_at | similar to px19-like protein | na |
| 1382049_at | similar to thymus atrophy-related protein | na |
| 1389907_at | similar to archease | na |
| 1375327_at | hypothetical LOC292596 | na |
| 1372668_at | similar to LIM domains containing protein 1 | na |
| 1380248_at | similar to semaphorin subclass 4 member G | na |
| 1374790_at | similar to hypothetical protein CAB56184 | na |
| 1373292_at | similar to KIAA0339 protein | na |
| 1374045_at | similar to Conserved oligomeric Golgi complex component 8 | na |
| 1372566_at | similar to mitochondrial carrier homolog 2 | na |
| 1389104_s_at | similar to zinc finger protein RIZ | na |
| 1399125_at | similar to inositol polyphosphate 1-phosphatase | na |
| 1398510_at | similar to KIAA1521 protein | na |
| 1380530_at | similar to mastermind homolog | na |
| 1374743_at | similar to InaD-like protein isoform 3; PDZ domain protein | na |
| | (Drosophila inaD-like); protein associated to tight junctions; | |
| | PALS1-associated tight junction protein; inactivation no after- | |
| | potential D-like protein | |
| 1382200_at | hypothetical LOC287390 | na |
| 1374465_at | similar to ubiquitously-expressed transcript isoform 1 | na |
| 1383443_at | similar to RIKEN cDNA 1110018J18 | na |
| 1385302_at | similar to RIKEN cDNA 2610020H15 | na |
| 1389571_at | similar to signal transducer and activator of transcription 2 | na |
| 1379515_at | similar to neuronal development-associated protein | na |
| 1388638_at | similar to Sirtuin 6 (silent mating type information regulation 2, | na |
| | homolog) 6 | |
| 1376573_at | similar to Rab family small GTPase Rah | na |
| 1385699_at | similar to Dermatomyositis associated with cancer putative | na |
| | autoantigen-1 homolog (DACA-1 homolog) | |
| 1374547_at | similar to RIKEN cDNA A930015D22 | na |
| 1370816_at | nuclear receptor subfamily 1, group D, member 1 | Nr1d1 |
| 1379481_at | poly(A) binding protein, nuclear 1 | Pabpn1 |
| 1387790_at | phosphoribosylaminoimidazole carboxylase, | Paics |
| | phosphoribosylaminoribosylaminoimidazole | |
| | succinocarboxamide synthetase | |
| 1368902_at | p21 (CDKN1A)-activated kinase 3 | Pak3 |
| 1367946_at | PDZ and LIM domain 1 | Pdlim1 |
| 1368655_at | proteoglycan peptide core protein | Pgsg |
| 1376190_at | phosphatidylinositol 3-kinase, regulatory subunit, polypeptide 2 | Pik3r2 |
| 1377077_at | plexin A3 | Plxna3 |
| 1370951_at | ER transmembrane protein Dri 42 | Ppap2b |
| 1388911_at | DNA primase, p58 subunit | Prim2 |
| 1369624_at | prolactin releasing hormone | Prlh |
| 1367851_at | prostaglandin D2 synthase | Ptgds |
| 1369614_at | RAP2B, member of RAS oncogene family | Rap2b |
| 1371583_at | RNA binding motif (RNP1, RRM) protein 3 | Rbm3 |
| 1394401_at | fatty acid elongase 2 | rELO2 |
| 1368144_at | regulator of G-protein signaling protein 2 | Rgs2 |
| 1388027_a_at | reticulon 4 | Rtn4 |
| 1387926_at | sterol-C5-desaturase (fungal ERG3, delta-5-desaturase)-like | Sc5d |
| 1369709_at | spinocerebellar ataxia 1 homolog (human) | Sca1 |
| 1387280_a_at | tumor-associated protein 1 | Slc7a5 |
| 1368599_at | solute carrier family 9, member 2 | Slc9a2 |
| 1398245_at | synuclein, gamma | Sncg |
| 1368439_at | SRY-box containing gene 10 | Sox10 |
| 1370420_at | steroid 5 alpha-reductase 1 | Srd5a1 |
| 1369718_at | TRAP-complex gamma subunit | Ssr3 |
| 1396512_at | synaptogyrin 1 | Syngr1 |
| 1387517_at | synaptotagmin 13 | Syt13 |
| 1390817_at | transcription factor 20 | Tcf20 |
| 1367859_at | transforming growth factor, beta 3 | Tgfb3 |
| 1374257_at | T-cell lymphoma invasion and metastasis 1 | Tiam1 |
| 1387370_at | tropomodulin 1 | Tmod1 |
| 1390217_at | tuberin-like protein 1 | Tulip1 |
| 1369597_at | vesicle-associated membrane protein, associated protein B and C | Vapb |
| 1372578_at | valyl-tRNA synthetase 2 | Vars2 |
| 1370828_at | zinc finger, DHHC domain containing 2 | Zdhhc2 |
| 1390003_at | zinc finger protein 57 | Zfp57 |

Genes and gene fragments identified as changing significantly in animals 0.5 hours following water maze training relative to controls are provided in Table 3 below.

TABLE 3

| Fragment Name | Gene Name | Gene Symbol |
|---|---|---|
| 1392616_at | (ERO1-like (S. cerevisiae), apoptotic protease activating factor | (Apaf1, Cln2, Ero11) |
| | 1, ceroid-lipofuscinosis, neuronal 2) | |
| 1383096_at | (amyloid beta (A4) precursor-like protein 2, sperm membrane | (Aplp2, LOC64312) |
| | protein (YWK-II)) | |
| 1376843_at | (bone morphogenic protein receptor, type II (serine/threonine | (Bmpr2, na) |
| | kinase), similar to Bone morphogenetic protein type II receptor) | |
| 1368072_at | (B-cell translocation gene 3, hypothetical gene supported by | (Btg3, na) |
| | NM_019290) | |
| 1369733_at | (beta-catenin, similar to beta-catenin; catenin beta) | (Catnb, na) |
| 1387436_at | (CDC10 (cell division cycle 10, S. cerevisiae, homolog), similar | (Cdc10, na) |
| | to cell division cycle 10 homolog) | |
| 1369351_at | (contactin 3, hypothetical gene supported by NM_019329) | (Cntn3, na) |
| 1387541_at | (chondroitin sulfate proteoglycan 3, hypothetical gene | (Cspg3, na) |
| | supported by NM_031653) | |
| 1389868_at | (DEAD (Asp-Glu-Ala-Asp) box polypeptide 6, trehalase | (Ddx6, Treh) |
| | (brush-border membrane glycoprotein)) | |
| 1370048_at | (endothelial differentiation, lysophosphatidic acid G-protein- | (Edg2, na) |
| | coupled receptor, 2, hypothetical gene supported by | |
| | NM_053936) | |
| 1398846_at | (eukaryotic translation initiation factor 5, similar to Eukaryotic | (Eif5, na) |
| | translation initiation factor 5 (eIF-5)) | |
| 1369640_at | (gap junction membrane channel protein alpha 1, hypothetical | (Gja1, na) |
| | gene supported by NM_012567) | |
| 1367732_at | (guanine nucleotide binding protein, beta 1, similar to guanine | (Gnb1, na) |
| | nucleotide-binding protein, beta-1 subunit) | |
| 1381677_at | (RNA binding protein HuB, hypothetical gene supported by | (Hub, na) |
| | NM_173309) | |
| 1381030_at | (similar to step II splicing factor SLU7; DNA segment, Chr 11, | (LOC303057, Slu7) |
| | ERATO Doi 730, expressed; DNA segment, Chr 3, Brigham & | |
| | Womens Genetics 0878 expressed, step II splicing factor SLU7 | |
| | (S. cerevisiae)) | |
| 1387071_a_at | (hypothetical gene supported by NM_017212, microtubule- | (Mapt, na) |
| | associated protein tau) | |
| 1370948_a_at | (myristoylated alanine rich protein kinase C substrate, similar to | (Marcks, na) |
| | Myristoylated alanine-rich C-kinase substrate (MARCKS)) | |
| 1373432_at | (myristoylated alanine rich protein kinase C substrate, similar to | (Marcks, na) |
| | Myristoylated alanine-rich C-kinase substrate (MARCKS)) | |
| 1370949_at | (myristoylated alanine rich protein kinase C substrate, similar to | (Marcks, na) |
| | Myristoylated alanine-rich C-kinase substrate (MARCKS)) | |
| 1370500_a_at | (hypothetical gene supported by NM_012720, myelin- | (Mobp, na) |
| | associated oligodendrocytic basic protein) | |
| 1368411_a_at | (hypothetical gene supported by NM_013066, microtubule- | (Mtap2, na) |
| | associated protein 2) | |
| 1371776_at | (hypothetical gene supported by NM_013005, | (Pik3r1, na) |
| | phosphatidylinositol 3-kinase, regulatory subunit, polypeptide | |
| | 1) | |
| 1375378_at | (homolog of mouse quaking QKI (KH domain RNA binding | (Qki, na) |
| | protein), similar to QKI isoform 7B) | |
| 1370745_at | (ribosomal protein L17, similar to 60S RIBOSOMAL | (Rpl17, na) |
| | PROTEIN L17 (L23) (AMINO ACID STARVATION- | |
| | INDUCED PROTEIN) (ASI), similar to Heph protein) | |
| 1388259_at | (SNRPN upstream reading frame, similar to small nuclear | (Snrpn, Snurf, na) |
| | ribonucleoprotein-associated protein N - rat, small nuclear | |
| | ribonucleoprotein N) | |
| 1386909_a_at | (similar to Voltage-dependent anion-selective channel protein 1 | (Vdac1, na) |
| | (VDAC-1) (mVDAC1) (mVDAC5) (Outer mitochondrial | |
| | membrane protein porin 1) (Plasmalemmal porin), voltage- | |
| | dependent anion channel 1) | |
| 1383053_x_at | (similar to zinc finger protein 91 isoform 1, zinc finger protein | (Zfp91, na) |
| | 91) | |
| 1383052_a_at | (similar to zinc finger protein 91 isoform 1, zinc finger protein | (Zfp91, na) |
| | 91) | |
| 1387054_at | ATP-binding cassette, sub-family G (WHITE), member 1 | Abcg1 |
| 1395886_at | ARP3 actin-related protein 3 homolog (yeast) | Actr3 |
| 1368933_at | adenosine deaminase, RNA-specific, B1 | Adarb1 |
| 1387302_at | adenylate cyclase activating polypeptide 1 receptor 1 | Adcyap1r1 |
| 1370043_at | activated leukocyte cell adhesion molecule | Alcam |
| 1387022_at | aldehyde dehydrogenase family 1, member A1 | Aldh1a1 |
| 1380533_at | amyloid beta (A4) precursor protein | App |
| 1374486_at | Rho guanine nucleotide exchange factor (GEF) 11 | Arhgef11 |
| 1375552_at | ADP-ribosylation factor-like 9 | Arl9 |
| 1370611_at | aryl hydrocarbon receptor nuclear translocator 2 | Arnt2 |
| 1393596_at | alpha thalassemia/mental retardation syndrome X-linked | Atrx |
| | (RAD54 homolog, S. cerevisiae) | |
| 1370113_at | inhibitor of apoptosis protein 1 | Birc3 |
| 1369248_a_at | baculoviral IAP repeat-containing 4 | Birc4 |
| 1368899_at | bone morphogenetic protein receptor, type 1A | Bmpr1a |
| 1387882_at | basic transcription element binding protein 1 | Bteb1 |
| 1369649_at | calcium channel, voltage-dependent, alpha2/delta subunit 1 | Cacna2d1 |
| 1375719_s_at | cadherin 13 | Cdh13 |
| 1369425_at | cadherin 13 | Cdh13 |
| 1380547_at | chloride channel 3 | Clcn3 |
| 1369609_at | claudin 11 | Cldn11 |
| 1387420_at | chloride intracellular channel 4 | Clic4 |
| 1369215_a_at | carboxypeptidase D | Cpd |
| 1368584_a_at | complexin 2 | Cplx2 |
| 1369912_at | v-crk sarcoma virus CT10 oncogene homolog (avian) | Crk |
| 1368849_at | casein kinase 1, gamma 3 | Csnk1g3 |
| 1387170_at | casein kinase II, alpha 1 polypeptide | Csnk2a1 |
| 1384339_s_at | casein kinase II, alpha 1 polypeptide | Csnk2a1 |
| 1375591_at | CUG triplet repeat, RNA-binding protein 2 | Cugbp2 |
| 1387328_at | Cytochrome P450, subfamily IIC (mephenytoin 4-hydroxylase) | Cyp2c |
| 1379480_at | diacylglycerol kinase, iota | Dgki |
| 1392196_at | discs, large (Drosophila) homolog 2 (chapsyn-110) | Dlg2 |
| 1398276_at | discs, large (Drosophila) homolog 2 (chapsyn-110) | Dlg2 |
| 1377300_at | membrane protein, palmitoylated 3 (MAGUK p55 subfamily | Dusp3 |
| | member 3) | |
| 1387024_at | dual specificity phosphatase 6 | Dusp6 |
| 1387146_a_at | endothelin receptor type B | Ednrb |
| 1374224_at | eukaryotic translation initiation factor 2 alpha kinase 4 | Eif2ak4 |
| 1368536_at | ectonucleotide pyrophosphatase/phosphodiesterase 2 | Enpp2 |
| 1369703_at | endothelial PAS domain protein 1 | Epas1 |
| 1374320_at | coagulation factor 5 | F5 |
| 1390682_at | rapostlin | Fnbp1 |
| 1387685_at | frequenin homolog (Drosophila) | Freq |
| 1369371_a_at | gamma-aminobutyric acid (GABA) B receptor, 1 | Gabbr1 |
| 1370760_a_at | glutamate decarboxylase 1 | Gad1 |
| 1387659_at | guanine deaminase | Gda |
| 1367633_at | glutamine synthetase 1 | Glul |
| 1387663_at | glia maturation factor, beta | Gmfb |
| 1387095_at | guanine nucleotide binding protein, alpha z subunit | Gnaz |
| 1369410_at | golgi SNAP receptor complex member 1 | Gosr1 |
| 1392125_at | glycosylphosphatidylinositol specific phospholipase D1 | Gpld1 |
| 1368985_at | glutamate receptor, ionotropic, N-methyl D-aspartate 2A | Grin2a |
| 1370125_at | hyaluronan and proteoglycan link protein 1 | Hapln1 |
| 1375335_at | heat shock 90 kDa protein 1, beta | Hspcb |
| 1368870_at | Inhibitor of DNA binding 2, dominant negative helix-loop-helix | Id2 |
| | protein | |
| 1370957_at | interleukin 6 signal transducer | Il6st |
| 1371148_s_at | internexin, alpha | Inexa |
| 1370232_at | isovaleryl coenzyme A dehydrogenase | Ivd |
| 1370975_at | jumonji domain containing 1A | Jmjd1a |
| 1387467_at | potassium inwardly-rectifying channel, subfamily J, member 10 | Kcnj10 |
| 1367948_a_at | kinase insert domain protein receptor | Kdr |
| 1391906_at | kinesin family member 1B | Kif1b |
| 1378509_at | kinesin family member 3a | Kif3a |
| 1382787_at | kinesin family member 5A | Kif5a |
| 1370035_at | Kirsten rat sarcoma viral oncogene homologue 2 (active) | Kras2 |
| 1387423_at | luteinizing hormone/choriogonadotropin receptor | Lhcgr |
| 1375726_at | LIM domain only protein 7 | LMO7 |
| 1387978_at | CTD-binding SR-like protein rA9 | LOC245925 |
| 1377080_at | p75-like apoptosis-inducing death domain protein PLAIDD | LOC246143 |
| 1388002_at | serine/threonine protein kinase TAO1 | LOC286993 |
| 1389876_at | CaM-kinase II inhibitor alpha | LOC287005 |
| 1370853_at | CaM-kinase II inhibitor alpha | LOC287005 |
| 1396036_at | Ral-A exchange factor RalGPS2 | LOC304887 |
| 1387649_at | CaM-KII inhibitory protein | LOC59314 |
| 1370550_at | limbic system-associated membrane protein | Lsamp |
| 1381821_at | MAM domain containing glycosylphosphatidylinositol anchor 2 | Mamdc1 |
| 1369820_at | MCF.2 cell line derived transforming sequence-like | Mcf2l |
| 1370831_at | monoglyceride lipase | Mgll |
| 1368450_at | myosin Va | Myo5a |
| 1376263_at | similar to 1810034B16Rik protein | na |
| 1375459_at | hypothetical gene supported by AF102149 | na |
| 1382024_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1383332_at | similar to nuclear body associated kinase 1a | na |
| 1392598_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1389514_at | similar to hypothetical protein FLJ31810 | na |
| 1396403_at | LOC361748 | na |
| 1382064_at | similar to 4933407C03Rik protein | na |
| 1393881_at | similar to N-terminal aceyltransferase 1 | na |
| 1395273_at | similar to hypothetical protein | na |
| 1385546_at | similar to PC326 protein | na |
| 1382785_at | similar to kinesin family protein KIF1a | na |
| 1395739_at | similar to RIKEN cDNA C920006C10 | na |
| 1379378_at | similar to RIKEN cDNA 1110037D14 gene | na |
| 1391416_at | similar to gene trap ankyrin repeat; serologically defined breast | na |
| | cancer antigen NY-BR-16 | |
| 1398476_at | similar to Vinculin (Metavinculin) | na |
| 1375650_at | similar to bromodomain-containing protein BRD4 short variant | na |
| 1375253_at | similar to nuclear factor, erythroid derived 2,-like 1 | na |
| 1379206_at | similar to RIKEN cDNA 1110001E17 | na |
| 1380235_at | similar to heparan sulfate 2-sulfotransferase | na |
| 1383455_at | similar to Bifunctional aminoacyl-tRNA synthetase | na |
| 1381789_at | similar to lysozyme homolog | na |
| 1377265_at | similar to holocytochrome c-type synthetase | na |
| 1384895_at | similar to KIAA0602 protein | na |
| 1397201_at | similar to KIAA1078 protein | na |
| 1390024_at | similar to osteoclast inhibitory lectin | na |
| 1374449_at | similar to Gene rich cluster, C8 gene | na |
| 1375026_at | similar to CLN6 protein | na |
| 1391117_at | similar to KIAA1183 protein | na |
| 1382561_at | similar to 4921517L17Rik protein | na |
| 1393795_at | similar to mKIAA0569 protein | na |
| 1397405_at | similar to Probable RNA-dependent helicase p72 (DEAD-box | na |
| | protein p72) (DEAD-box protein 17) | |
| 1385491_at | similar to KIAA1183 protein | na |
| 1376619_at | similar to protein tyrosine phosphatase, receptor type, D | na |
| | isoform 3 precursor; protein tyrosine phosphatase, receptor | |
| | type, delta polypeptide; protein tyrosine phosphatase delta | |
| 1398420_at | similar to E3 ubiquitin ligase SMURF2 | na |
| 1383013_at | similar to Krueppel-like factor 13 (Transcription factor BTEB3) | na |
| | (Basic transcription element binding protein 3) (BTE-binding | |
| | protein 3) (RANTES factor of late activated T lymphocytes-1) | |
| | (RFLAT-1) (Transcription factor NSLP1) (Novel Sp1-like zinc | |
| | fi . . . | |
| 1397959_at | similar to RIKEN cDNA D130059P03 gene | na |
| 1377814_at | similar to Nedd4-binding brain specific protein BEAN | na |
| 1391578_at | similar to modulator of estrogen induced transcription | na |
| 1393932_at | similar to RIKEN cDNA 1700010P07 | na |
| 1398062_at | similar to hypothetical protein AL133206 | na |
| 1374047_at | similar to RIKEN cDNA 2400010D15 | na |
| 1378740_at | similar to Ras GTPase-activating protein nGAP (RAS protein | na |
| | activator like 1) | |
| 1391817_at | similar to RIKEN cDNA 6330416G13 gene | na |
| 1382963_at | similar to ABC transporter | na |
| 1391336_at | similar to I-kappa-B-interacting Ras-like protein 2 | na |
| 1372385_at | similar to neuronal tyrosine threonine phosphatase 1 | na |
| 1378690_at | similar to Ly6-B antigen gene | na |
| 1383097_at | similar to RIKEN cDNA 2010012O05 | na |
| 1380172_at | similar to mKIAA0531 protein | na |
| 1391170_at | (similar to Gene trap ROSA 26 antisense, Philippe Soriano, | na |
| | similar to mKIAA1757 protein) | |
| 1375231_a_at | similar to RIKEN cDNA 4930415K17 | na |
| 1377434_at | similar to membrane-type frizzled-related protein | na |
| 1392051_at | similar to hypothetical protein FLJ14681 | na |
| 1381542_at | similar to RIKEN cDNA 1300013G12 | na |
| 1389060_at | similar to SH2 domain containing SOCS box protein SOCS7 | na |
| 1391618_at | similar to KAP3A | na |
| 1395418_at | similar to putative transcription factor ZNF131 | na |
| 1371980_at | similar to TOB3 | na |
| 1398581_at | similar to grey lethal osteopetrosis; grey-lethal; grey lethal | na |
| | osteroperosis | |
| 1376456_at | similar to nicotinamide mononucleotide adenylyltransferase 2 | na |
| | isoform 1; pyridine nucleotide adenylyltransferase 2; | |
| | chromosome 1 open reading frame 15 | |
| 1380446_at | similar to AF-10 protein | na |
| 1393764_at | similar to opioid growth factor receptor | na |
| 1377174_at | similar to SET binding factor 1 (Sbf1) (Myotubularin-related | na |
| | protein 5) | |
| 1375022_at | similar to 2310036I02Rik protein | na |
| 1375177_at | similar to Krueppel-like factor 13 (Transcription factor BTEB3) | na |
| | (Basic transcription element binding protein 3) (BTE-binding | |
| | protein 3) (RANTES factor of late activated T lymphocytes-1) | |
| | (RFLAT-1) (Transcription factor NSLP1) (Novel Sp1-like zinc | |
| | fi . . . | |
| 1383631_at | similar to Oligosaccharyl transferase 3 CG7748-PA | na |
| 1394746_at | similar to guanine nucleotide exchange factor p532 | na |
| 1372781_at | similar to RIKEN cDNA 1810073M12 | na |
| 1397618_at | similar to JNK-associated leucine-zipper protein | na |
| 1379765_at | (similar to nemo-like kinase, similar to nemo-like kinase; likely | na |
| | ortholog of mouse nemo like kinase) | |
| 1396629_at | similar to ORM1-like 3 | na |
| 1383615_a_at | similar to E3 ligase for inhibin receptor | na |
| 1398065_at | similar to hypothetical protein FLJ10159 | na |
| 1377167_at | similar to Phosphoinositol 4-phosphate Adaptor Protein-1 | na |
| 1375278_at | similar to KIAA0517 protein | na |
| 1372517_at | similar to Peptidyl-prolyl cis-trans isomerase like 1 (PPIase) | na |
| | (Rotamase) | |
| 1391747_at | similar to copine I | na |
| 1391075_at | similar to regulator of G-protein signaling Z2 | na |
| 1376645_at | similar to RIKEN cDNA 6330406I15 | na |
| 1391688_at | similar to NF-kappaB inducing kinase | na |
| 1375703_at | similar to MLL5 | na |
| 1377654_at | similar to RIKEN cDNA 1810037C20 | na |
| 1384280_at | similar to nucleolar protein ANKT | na |
| 1375528_at | similar to intracellular membrane-associated calcium- | na |
| | independent phospholipase A2 gamma | |
| 1376085_at | similar to family with sequence similarity 11 member A | na |
| 1376256_at | similar to WD repeat and FYVE domain containing 1 isoform | na |
| | 1; phosphoinositide-binding protein SR1; WD40 and FYVE | |
| | domain containing 1 | |
| 1372536_at | similar to Cabc1 protein | na |
| 1377137_at | similar to tyrosyl-DNA phodphodiesterase 1 | na |
| 1397628_at | similar to RIKEN cDNA 2900092E17 | na |
| 1371349_at | similar to collagen alpha1 type VI-precursor | na |
| 1398958_at | similar to RIKEN cDNA 9030612I22 | na |
| 1373630_at | similar to intermediate filament-like protein MGC: 2625 isoform | na |
| | 2; HOM-TES-103 tumor antigen-like | |
| 1391512_at | similar to myotubularin related protein 1 isoform A | na |
| 1373925_at | similar to putative lipid kinase | na |
| 1392864_at | similar to p190-B | na |
| 1375280_at | similar to 5830417C01Rik protein | na |
| 1394445_at | similar to mKIAA0515 protein | na |
| 1378246_at | similar to T-cell activation protein phosphatase 2C | na |
| 1397194_at | similar to CG5554-PA | na |
| 1382556_a_at | similar to DIP13 alpha | na |
| 1382945_at | similar to cDNA sequence BC006662 | na |
| 1382261_at | similar to CG8841-PA | na |
| 1376299_at | similar to Retinoblastoma-binding protein 2 (RBBP-2) | na |
| 1395343_at | similar to transmembrane protein TM9SF3 | na |
| 1385130_at | similar to fez-like | na |
| 1386535_at | similar to RNA helicase | na |
| 1374743_at | similar to InaD-like protein isoform 3; PDZ domain protein | na |
| | (Drosophila inaD-like); protein associated to tight junctions; | |
| | PALS1-associated tight junction protein; inactivation no after- | |
| | potential D-like protein | |
| 1395940_at | similar to 1-acylglycerolphosphate acyltransferase-epsilon | na |
| 1376449_at | similar to hypothetical protein FLJ12770 | na |
| 1385594_at | similar to mitochondrial ribosomal protein L51; mitochondrial | na |
| | ribosomal protein 64 | |
| 1387204_at | neuronal growth regulator 1 | Negr1 |
| 1370946_at | nuclear factor I/X | Nfix |
| 1387409_x_at | neuroligin 3 | Nlgn3 |
| 1376734_at | nephroblastoma overexpressed gene | Nov |
| 1368972_at | neurotrophic tyrosine kinase, receptor, type 2 | Ntrk2 |
| 1370687_a_at | neurotrophic tyrosine kinase, receptor, type 2 | Ntrk2 |
| 1375765_at | neural visinin-like Ca2+-binding protein type 2 | Nvjp2 |
| 1391625_at | neural Wiskott-Aldrich syndrome protein | N-WASP |
| 1369646_at | opioid receptor-like | Oprl |
| 1368958_at | protein kinase C and casein kinase substrate in neurons 1 | Pacsin1 |
| 1382943_at | protocadherin gamma subfamily C, 3 | Pcdhgc3 |
| 1382303_at | phosphatase and actin regulator 1 | Phactr1 |
| 1370950_at | ER transmembrane protein Dri 42 | Ppap2b |
| 1370951_at | ER transmembrane protein Dri 42 | Ppap2b |
| 1370552_at | protein phosphatase 1F (PP2C domain containing) | Ppm1f |
| 1368240_a_at | protein kinase C, beta 1 | Prkcb1 |
| 1374593_at | protein kinase C, epsilon | Prkce |
| 1367710_at | protease (prosome, macropain) 28 subunit, beta | Psme2 |
| 1367884_at | RAB14, member RAS oncogene family | Rab14 |
| 1391599_at | RAB14, member RAS oncogene family | Rab14 |
| 1370780_at | RAB31, member RAS oncogene family | Rab31 |
| 1375360_at | Ras homolog enriched in brain | Rheb |
| 1388071_x_at | RT1 class Ib, locus Aw2 | RT1-Aw2 |
| 1386889_at | stearoyl-Coenzyme A desaturase 2 | Scd2 |
| 1388253_at | stearoyl-Coenzyme A desaturase 2 | Scd2 |
| 1369092_at | sec22 homolog | Sec22l2 |
| 1368986_at | solute carrier family 17 (sodium-dependent inorganic phosphate | Slc17a7 |
| | cotransporter), member 7 | |
| 1387707_at | solute carrier family 2 (facilitated glucose transporter), member 3 | Slc2a3 |
| 1369756_a_at | solute carrier family 4, member 4 | Slc4a4 |
| 1392349_at | solute carrier family 5 (inositol transporters), member 3 | Slc5a3 |
| 1394205_at | serine/threonine kinase 2 | Slk |
| 1379281_at | uterine sensitization-associated gene 1 protein | Sostdc1 |
| 1390706_at | spectrin beta 2 | Spnb2 |
| 1369718_at | TRAP-complex gamma subunit | Ssr3 |
| 1368842_at | transcription factor 4 | Tcf4 |
| 1369919_at | thyrotroph embryonic factor | Tef |
| 1370691_a_at | thyroid hormone receptor alpha | Thra |
| 1393418_at | tropomodulin 2 | Tmod2 |
| 1369541_at | tropomodulin 2 | Tmod2 |
| 1391535_at | thioredoxin-like (32 kD) | Txnl1 |
| 1369597_at | vesicle-associated membrane protein, associated protein B and C | Vapb |

Genes and gene fragments identified as changing significantly in animals 1.0 hour following water maze training relative to controls are provided in Table 4 below.

TABLE 4

| Fragment Name | Gene Name | Gene Symbol |
|---|---|---|
| 1367732_at | (guanine nucleotide binding protein, beta 1, similar to guanine | (Gnb1, na) |
| | nucleotide-binding protein, beta-1 subunit) | |
| 1367553_x_at | (hemoglobin beta chain complex, similar to Hemoglobin beta | (Hbb, na) |
| | chain, minor-form) | |
| 1371003_at | (microtubule-associated protein 1b, similar to Microtubule- | (Map1b, na) |
| | associated protein 1B (MAP 1B) (Neuraxin)) | |
| 1370948_a_at | (myristoylated alanine rich protein kinase C substrate, similar to | (Marcks, na) |
| | Myristoylated alanine-rich C-kinase substrate (MARCKS)) | |
| 1383052_a_at | (similar to zinc finger protein 91 isoform 1, zinc finger protein | (Zfp91, na) |
| | 91) | |
| 1370842_at | branched chain keto acid dehydrogenase kinase | Bckdk |
| 1367821_a_at | calcium channel, voltage-dependent, P/Q type, alpha 1A | Cacna1a |
| | subunit | |
| 1388187_at | calcium/calmodulin-dependent protein kinase II alpha subunit | Camk2a |
| 1387170_at | casein kinase II, alpha 1 polypeptide | Csnk2a1 |
| 1375462_at | discoidin domain receptor family, member 1 | Ddr1 |
| 1392196_at | discs, large (Drosophila) homolog 2 (chapsyn-110) | Dlg2 |
| 1398276_at | discs, large (Drosophila) homolog 2 (chapsyn-110) | Dlg2 |
| 1387210_at | discs, large homolog 4 (Drosophila) | Dlgh4 |
| 1368146_at | dual specificity phosphatase 1 | Dusp1 |
| 1384687_at | ectodermal-neural cortex 1 | Enc1 |
| 1368536_at | ectonucleotide pyrophosphatase/phosphodiesterase 2 | Enpp2 |
| 1374320_at | coagulation factor 5 | F5 |
| 1387889_at | folate receptor 1 (adult) | Folr1 |
| 1387685_at | frequenin homolog (Drosophila) | Freq |
| 1369371_a_at | gamma-aminobutyric acid (GABA) B receptor, 1 | Gabbr1 |
| 1387095_at | guanine nucleotide binding protein, alpha z subunit | Gnaz |
| 1387670_at | glycerol-3-phosphate dehydrogenase 2 | Gpd2 |
| 1370344_at | heat shock 70 kDa protein 4 | Hspa4 |
| 1367571_a_at | insulin-like growth factor 2 | Igf2 |
| 1370651_a_at | inositol polyphosphate phosphatase-like 1 | Inppl1 |
| 1382787_at | kinesin family member 5A | Kif5a |
| 1370853_at | CaM-kinase II inhibitor alpha | LOC287005 |
| 1368861_a_at | myelin-associated glycoprotein | Mag |
| 1367682_at | midkine | Mdk |
| 1368302_at | homeo box, msh-like 1 | Msx1 |
| 1375648_at | similar to hypothetical protein | na |
| 1376263_at | similar to 1810034B16Rik protein | na |
| 1396173_at | similar to D7H11orf14 protein | na |
| 1382024_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1394668_at | similar to nectin-like protein 1 | na |
| 1395409_at | similar to protein phosphatase 2A, regulatory subunit B (PR 53) | na |
| 1392598_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1378520_at | similar to zinc finger protein mRit1 beta | na |
| 1382785_at | similar to kinesin family protein KIF1a | na |
| 1399118_at | similar to RIKEN cDNA 4121402D02 | na |
| 1375253_at | similar to nuclear factor, erythroid derived 2,-like 1 | na |
| 1385601_at | similar to FSHD region gene 1 | na |
| 1377434_at | similar to membrane-type frizzled-related protein | na |
| 1372361_at | similar to DXImx40e protein | na |
| 1379904_at | similar to hypothetical protein MGC2650 | na |
| 1379221_at | similar to SP1a/RYanodine receptor SPRY (1J970) | na |
| 1373179_at | similar to mKIAA1107 protein | na |
| 1397659_at | similar to hypothetical protein 2810027J07 | na |
| 1391855_at | similar to B-cell CLL/lymphoma 7A | na |
| 1371849_at | similar to hypothetical protein FLJ12442 | na |
| 1383231_at | similar to Smad nuclear interacting protein | na |
| 1375687_at | RAB14, member RAS oncogene family | Rab14 |
| 1368986_at | solute carrier family 17 (sodium-dependent inorganic phosphate | Slc17a7 |
| | cotransporter), member 7 | |
| 1368440_at | solute carrier family 3, member 1 | Slc3a1 |
| 1387280_a_at | tumor-associated protein 1 | Slc7a5 |
| 1368606_at | solute carrier organic anion transporter family, member 1A2 | Slco1a2 |
| 1379281_at | uterine sensitization-associated gene 1 protein | Sostdc1 |
| 1369718_at | TRAP-complex gamma subunit | Ssr3 |
| 1369712_at | serine/threonine kinase 3 | Stk3 |
| 1374257_at | T-cell lymphoma invasion and metastasis 1 | Tiam1 |
| 1369541_at | tropomodulin 2 | Tmod2 |
| 1367598_at | transthyretin | Ttr |

Genes and gene fragments identified as changing significantly in animals 2 hours following water maze training relative to controls are provided in Table 5 below.

TABLE 5

| Fragment Name | Gene Name | Gene Symbol |
|---|---|---|
| 1383096_at | (amyloid beta (A4) precursor-like protein 2, sperm membrane | (Aplp2, LOC64312) |
| | protein (YWK-II)) | |
| 1376843_at | (bone morphogenic protein receptor, type II (serine/threonine | (Bmpr2, na) |
| | kinase), similar to Bone morphogenetic protein type II receptor) | |
| 1369819_at | (bassoon, hypothetical gene supported by NM_019146) | (Bsn, na) |
| 1369733_at | (beta-catenin, similar to beta-catenin; catenin beta) | (Catnb, na) |
| 1373067_at | (beta-catenin, similar to beta-catenin; catenin beta) | (Catnb, na) |
| 1387436_at | (CDC10 (cell division cycle 10, S. cerevisiae, homolog), similar | (Cdc10, na) |
| | to cell division cycle 10 homolog) | |
| 1389868_at | (DEAD (Asp-Glu-Ala-Asp) box polypeptide 6, trehalase | (Ddx6, Treh) |
| | (brush-border membrane glycoprotein)) | |
| 1370048_at | (endothelial differentiation, lysophosphatidic acid G-protein- | (Edg2, na) |
| | coupled receptor, 2, hypothetical gene supported by | |
| | NM_053936) | |
| 1367732_at | (guanine nucleotide binding protein, beta 1, similar to guanine | (Gnb1, na) |
| | nucleotide-binding protein, beta-1 subunit) | |
| 1370948_a_at | (myristoylated alanine rich protein kinase C substrate, similar to | (Marcks, na) |
| | Myristoylated alanine-rich C-kinase substrate (MARCKS)) | |
| 1370949_at | (myristoylated alanine rich protein kinase C substrate, similar to | (Marcks, na) |
| | Myristoylated alanine-rich C-kinase substrate (MARCKS)) | |
| 1368411_a_at | (hypothetical gene supported by NM_013066, microtubule- | (Mtap2, na) |
| | associated protein 2) | |
| 1371776_at | (hypothetical gene supported by NM_013005, | (Pik3r1, na) |
| | phosphatidylinositol 3-kinase, regulatory subunit, polypeptide | |
| | 1) | |
| 1383421_at | (prostaglandin E receptor 1, protein kinase N1) | (Pkn1, Ptger1) |
| 1375378_at | (homolog of mouse quaking QKI (KH domain RNA binding | (Qki, na) |
| | protein), similar to QKI isoform 7B) | |
| 1391078_at | (replication factor C, similar to replication factor C) | (Recc1, na) |
| 1369691_at | (hypothetical gene supported by NM_013119, sodium channel, | (Scn3a, na) |
| | voltage-gated, type III, alpha polypeptide) | |
| 1386909_a_at | (similar to Voltage-dependent anion-selective channel protein 1 | (Vdac1, na) |
| | (VDAC-1) (mVDAC1) (mVDAC5) (Outer mitochondrial | |
| | membrane protein porin 1) (Plasmalemmal porin), voltage- | |
| | dependent anion channel 1) | |
| 1383054_at | (similar to zinc finger protein 91 isoform 1, zinc finger protein | (Zfp91, na) |
| | 91) | |
| 1383053_x_at | (similar to zinc finger protein 91 isoform 1, zinc finger protein | (Zfp91, na) |
| | 91) | |
| 1383052_a_at | (similar to zinc finger protein 91 isoform 1, zinc finger protein | (Zfp91, na) |
| | 91) | |
| 1387302_at | adenylate cyclase activating polypeptide 1 receptor 1 | Adcyap1r1 |
| 1382981_at | Abelson helper integration site 1 | Ahi1 |
| 1371132_a_at | ankyrin 3 (G) | Ank3 |
| 1380533_at | amyloid beta (A4) precursor protein | App |
| 1369159_at | androgen receptor | Ar |
| 1387447_at | ADP-ribosylation factor 3 | Arf3 |
| 1378372_at | arginase 2 | Arg2 |
| 1397853_s_at | Cdc42 guanine nucleotide exchange factor (GEF) 9 | Arhgef9 |
| 1390100_s_at | HLA-B-associated transcript 1A | Bat1a |
| 1369248_a_at | baculoviral IAP repeat-containing 4 | Birc4 |
| 1367821_a_at | calcium channel, voltage-dependent, P/Q type, alpha 1A | Cacna1a |
| | subunit | |
| 1369649_at | calcium channel, voltage-dependent, alpha2/delta subunit 1 | Cacna2d1 |
| 1388187_at | calcium/calmodulin-dependent protein kinase II alpha subunit | Camk2a |
| 1370438_at | C-terminal PDZ domain ligand of neuronal nitric oxide | Capon |
| | synthase | |
| 1369559_a_at | CD47 antigen (Rh-related antigen, integrin-associated signal | Cd47 |
| | transducer) | |
| 1368584_a_at | complexin 2 | Cplx2 |
| 1387170_at | casein kinase II, alpha 1 polypeptide | Csnk2a1 |
| 1384339_s_at | casein kinase II, alpha 1 polypeptide | Csnk2a1 |
| 1392196_at | discs, large (Drosophila) homolog 2 (chapsyn-110) | Dlg2 |
| 1398276_at | discs, large (Drosophila) homolog 2 (chapsyn-110) | Dlg2 |
| 1387210_at | discs, large homolog 4 (Drosophila) | Dlgh4 |
| 1371167_at | membrane protein, palmitoylated 3 (MAGUK p55 subfamily | Dusp3 |
| | member 3) | |
| 1368866_at | GERp95 | Eif2c2 |
| 1368536_at | ectonucleotide pyrophosphatase/phosphodiesterase 2 | Enpp2 |
| 1371007_at | EphA5 | Epha5 |
| 1374320_at | coagulation factor 5 | F5 |
| 1368011_at | ferredoxin reductase | Fdxr |
| 1367700_at | fibromodulin | Fmod |
| 1387889_at | folate receptor 1 (adult) | Folr1 |
| 1387685_at | frequenin homolog (Drosophila) | Freq |
| 1369371_a_at | gamma-aminobutyric acid (GABA) B receptor, 1 | Gabbr1 |
| 1367633_at | glutamine synthetase 1 | Glul |
| 1387095_at | guanine nucleotide binding protein, alpha z subunit | Gnaz |
| 1368985_at | glutamate receptor, ionotropic, N-methyl D-aspartate 2A | Grin2a |
| 1369745_at | glutamate receptor, ionotropic, NMDA2B | Grin2b |
| 1382492_a_at | hydroxysteroid (17-beta) dehydrogenase 8 | Hsd17b8 |
| 1368870_at | Inhibitor of DNA binding 2, dominant negative helix-loop-helix | Id2 |
| | protein | |
| 1375532_at | Inhibitor of DNA binding 2, dominant negative helix-loop-helix | Id2 |
| | protein | |
| 1367571_a_at | insulin-like growth factor 2 | Igf2 |
| 1367648_at | insulin-like growth factor binding protein 2 | Igfbp2 |
| 1387689_at | potassium voltage-gated channel, shaker-related subfamily, beta | Kcnab2 |
| | member 2 | |
| 1369133_a_at | potassium voltage gated channel, Shaw-related subfamily, | Kcnc3 |
| | member 3 | |
| 1387946_at | lectin, galactoside-binding, soluble, 3 binding protein | Lgals3bp |
| 1388002_at | serine/threonine protein kinase TAO1 | LOC286993 |
| 1389876_at | CaM-kinase II inhibitor alpha | LOC287005 |
| 1370853_at | CaM-kinase II inhibitor alpha | LOC287005 |
| 1391491_a_at | similar to MHR23B | LOC298012 |
| 1397568_at | hypothetical protein | LOC362246 |
| 1367682_at | midkine | Mdk |
| 1370831_at | monoglyceride lipase | Mgll |
| 1370301_at | matrix metalloproteinase 2 (72 KDa type IV collagenase) | Mmp2 |
| 1368302_at | homeo box, msh-like 1 | Msx1 |
| 1390423_at | MYC binding protein 2 | Mycbp2 |
| 1376263_at | similar to 1810034B16Rik protein | na |
| 1396173_at | similar to D7H11orf14 protein | na |
| 1382024_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1394668_at | similar to nectin-like protein 1 | na |
| 1383332_at | similar to nuclear body associated kinase 1a | na |
| 1395409_at | similar to protein phosphatase 2A, regulatory subunit B (PR 53) | na |
| 1392598_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1396403_at | LOC361748 | na |
| 1384388_at | similar to gp25L2 protein0 | na |
| 1385546_at | similar to PC326 protein | na |
| 1378520_at | similar to zinc finger protein mRit1 beta | na |
| 1379101_at | similar to DEAD/H box polypeptide 36 protein | na |
| 1375650_at | similar to bromodomain-containing protein BRD4 short variant | na |
| 1375253_at | similar to nuclear factor, erythroid derived 2,-like 1 | na |
| 1377457_a_at | similar to gp250 precursor | na |
| 1379206_at | similar to RIKEN cDNA 1110001E17 | na |
| 1395454_at | similar to grey lethal osteopetrosis; grey-lethal; grey lethal | na |
| | osteroperosis | |
| 1380235_at | similar to heparan sulfate 2-sulfotransferase | na |
| 1389433_at | similar to McKusick-Kaufman syndrome protein; MKKS | na |
| | protein | |
| 1391043_at | similar to C15orf16 protein | na |
| 1385601_at | similar to FSHD region gene 1 | na |
| 1391117_at | similar to KIAA1183 protein | na |
| 1382561_at | similar to 4921517L17Rik protein | na |
| 1393795_at | similar to mKIAA0569 protein | na |
| 1385491_at | similar to KIAA1183 protein | na |
| 1393257_at | similar to CUG triplet repeat RNA-binding protein 1 (CUG- | na |
| | BP1) (RNA-binding protein BRUNOL-2) (Deadenylation | |
| | factor CUG-BP) (Deadenylation factor EDEN-BP) (Brain | |
| | protein F41) | |
| 1398420_at | similar to E3 ubiquitin ligase SMURF2 | na |
| 1385044_at | similar to TGF-beta induced apotosis protein 2 | na |
| 1382963_at | similar to ABC transporter | na |
| 1385227_at | similar to Zinc finger transcription factor Trps1 (Zinc finger | na |
| | protein GC79) (Tricho-rhino-phalangeal syndrome type I | |
| | protein) | |
| 1375538_at | similar to Vinculin (Metavinculin) | na |
| 1374004_at | similar to RIKEN cDNA D030028O16 | na |
| 1391170_at | (similar to Gene trap ROSA 26 antisense, Philippe Soriano, | na |
| | similar to mKIAA1757 protein) | |
| 1379645_at | similar to polybromo-1D | na |
| 1377434_at | similar to membrane-type frizzled-related protein | na |
| 1381542_at | similar to RIKEN cDNA 1300013G12 | na |
| 1389060_at | similar to SH2 domain containing SOCS box protein SOCS7 | na |
| 1373790_at | similar to Aph1a-pending protein | na |
| 1391618_at | similar to KAP3A | na |
| 1395418_at | similar to putative transcription factor ZNF131 | na |
| 1379123_at | similar to Type I transmembrane receptor (seizure-related | na |
| | protein) | |
| 1393620_at | similar to Sestrin 3 | na |
| 1388299_at | similar to Cytosolic acyl coenzyme A thioester hydrolase, | na |
| | inducible (Long chain acyl-CoA thioester hydrolase) (Long | |
| | chain acyl-CoA hydrolase) (CTE-I) (LACH2) (ACH2) | |
| 1380446_at | similar to AF-10 protein | na |
| 1393764_at | similar to opioid growth factor receptor | na |
| 1375177_at | similar to Krueppel-like factor 13 (Transcription factor BTEB3) | na |
| | (Basic transcription element binding protein 3) (BTE-binding | |
| | protein 3) (RANTES factor of late activated T lymphocytes-1) | |
| | (RFLAT-1) (Transcription factor NSLP1) (Novel Sp1-like zinc | |
| | fi . . . | |
| 1374094_at | similar to 1500031K13Rik protein | na |
| 1397618_at | similar to JNK-associated leucine-zipper protein | na |
| 1393564_at | similar to zinc finger DAZ interacting protein 3 | na |
| 1371256_at | similar to protein tyrosine phosphatase 20 | na |
| 1384480_at | similar to RIKEN cDNA 1810048J11 | na |
| 1372361_at | similar to DXImx40e protein | na |
| 1376146_at | similar to RIKEN cDNA 2310033P09 | na |
| 1375703_at | similar to MLL5 | na |
| 1384728_at | similar to checkpoint suppressor 1 | na |
| 1389705_at | similar to recombination activating gene 1 gene activation | na |
| 1383897_at | similar to apoptosis-inducing factor (AIF)-homologous | na |
| | mitochondrion-associated inducer of death; p53-responsive | |
| | gene 3 | |
| 1398472_at | similar to Twinkle | na |
| 1382191_at | similar to Protein inhibitor of activated STAT protein 1 | na |
| | (DEAD/H box binding protein 1) | |
| 1376861_at | similar to RIKEN cDNA 1810018L05 | na |
| 1379317_a_at | similar to RRP5 protein homolog (Programmed cell death | na |
| | protein 11) | |
| 1370946_at | nuclear factor I/X | Nfix |
| 1370669_a_at | phosphodiesterase 10A | Pde10a |
| 1369882_at | prodynorphin | Pdyn |
| 1382303_at | phosphatase and actin regulator 1 | Phactr1 |
| 1382307_at | protein phosphatase 1, regulatory (inhibitor) subunit 12A | Ppp1r12a |
| 1385314_at | RAB14, member RAS oncogene family | Rab14 |
| 1375687_at | RAB14, member RAS oncogene family | Rab14 |
| 1370758_at | RAB15, member RAS onocogene family | Rab15 |
| 1396207_at | radixin | Rdx |
| 1388027_a_at | reticulon 4 | Rtn4 |
| 1387926_at | sterol-C5-desaturase (fungal ERG3, delta-5-desaturase)-like | Sc5d |
| 1368986_at | solute carrier family 17 (sodium-dependent inorganic phosphate | Slc17a7 |
| | cotransporter), member 7 | |
| 1387707_at | solute carrier family 2 (facilitated glucose transporter), member 3 | Slc2a3 |
| 1368440_at | solute carrier family 3, member 1 | Slc3a1 |
| 1368082_at | solute carrier family 4, member 2 | Slc4a2 |
| 1392349_at | solute carrier family 5 (inositol transporters), member 3 | Slc5a3 |
| 1369715_at | solute carrier family 6 (neurotransmitter transporter, GABA), | Slc6a11 |
| | member 11 | |
| 1387280_a_at | tumor-associated protein 1 | Slc7a5 |
| 1368606_at | solute carrier organic anion transporter family, member 1A2 | Slco1a2 |
| 1371004_at | sortilin 1 | Sort1 |
| 1379281_at | uterine sensitization-associated gene 1 protein | Sostdc1 |
| 1369718_at | TRAP-complex gamma subunit | Ssr3 |
| 1367762_at | somatostatin | Sst |
| 1368842_at | transcription factor 4 | Tcf4 |
| 1392382_at | transforming growth factor, beta 2 | Tgfb2 |
| 1374257_at | T-cell lymphoma invasion and metastasis 1 | Tiam1 |
| 1369541_at | tropomodulin 2 | Tmod2 |
| 1369692_at | tenascin R | Tnr |
| 1367598_at | transthyretin | Ttr |
| 1370556_at | vesicle-associated membrane protein 1 | Vamp1 |
| 1369597_at | vesicle-associated membrane protein, associated protein B and C | Vapb |

Genes and gene fragments identified as changing significantly in animals 3 hours following water maze training relative to controls are provided in Table 6 below.

TABLE 6

| Fragment Name | Gene Name | Gene Symbol |
|---|---|---|
| 1376843_at | (bone morphogenic protein receptor, type II (serine/threonine | (Bmpr2, na) |
| | kinase), similar to Bone morphogenetic protein type II receptor) | |
| 1370810_at | (cyclin D2, similar to G1/S-specific cyclin D2 (VIN-1 proto- | (Ccnd2, na) |
| | oncogene)) | |
| 1381030_at | (similar to step II splicing factor SLU7; DNA segment, Chr 11, | (LOC303057, Slu7) |
| | ERATO Doi 730, expressed; DNA segment, Chr 3, Brigham & | |
| | Womens Genetics 0878 expressed, step II splicing factor SLU7 | |
| | (S. cerevisiae)) | |
| 1398588_at | (similar to step II splicing factor SLU7; DNA segment, Chr 11, | (LOC303057, Slu7) |
| | ERATO Doi 730, expressed; DNA segment, Chr 3, Brigham & | |
| | Womens Genetics 0878 expressed, step II splicing factor SLU7 | |
| | (S. cerevisiae)) | |
| 1387071_a_at | (hypothetical gene supported by NM_017212, microtubule- | (Mapt, na) |
| | associated protein tau) | |
| 1383053_x_at | (similar to zinc finger protein 91 isoform 1, zinc finger protein | (Zfp91, na) |
| | 91) | |
| 1377105_at | (similar to zinc finger protein 91 isoform 1, zinc finger protein (Zfp91, na) | |
| | 91) | |
| 1369341_a_at | activin receptor interacting protein 1 | Acvrip1 |
| 1387837_at | adenomatosis polyposis coli | Apc |
| 1387199_a_at | Cdc42 guanine nucleotide exchange factor (GEF) 9 | Arhgef9 |
| 1375552_at | ADP-ribosylation factor-like 9 | Arl9 |
| 1387317_at | arginine vasopressin | Avp |
| 1369248_a_at | baculoviral IAP repeat-containing 4 | Birc4 |
| 1369649_at | calcium channel, voltage-dependent, alpha2/delta subunit 1 | Cacna2d1 |
| 1387428_at | calcium channel, voltage-dependent, beta 1 subunit | Cacnb1 |
| 1387133_at | calbindin 2 | Calb2 |
| 1398251_a_at | calcium/calmodulin-dependent protein kinase II beta subunit | Camk2b |
| 1375719_s_at | cadherin 13 | Cdh13 |
| 1370596_a_at | SH3 domain binding protein CR16 | Cr16 |
| 1384339_s_at | casein kinase II, alpha 1 polypeptide | Csnk2a1 |
| 1370053_at | discs, large (Drosophila) homolog-associated protein 1 | Dlgap1 |
| 1387210_at | discs, large homolog 4 (Drosophila) | Dlgh4 |
| 1382778_at | dual specificity phosphatase 6 | Dusp6 |
| 1384687_at | ectodermal-neural cortex 1 | Enc1 |
| 1368536_at | ectonucleotide pyrophosphatase/phosphodiesterase 2 | Enpp2 |
| 1372564_at | v-ets erythroblastosis virus E26 oncogene homolog 2 (avian) | Ets2 |
| 1368473_at | gap junction membrane channel protein alpha 5 | Gja5 |
| 1368952_at | G protein-coupled receptor 51 | Gpr51 |
| 1368401_at | glutamate receptor, ionotropic, 2 | Gria2 |
| 1368985_at | glutamate receptor, ionotropic, N-methyl D-aspartate 2A | Grin2a |
| 1370344_at | heat shock 70 kDa protein 4 | Hspa4 |
| 1375335_at | heat shock 90 kDa protein 1, beta | Hspcb |
| 1371148_s_at | internexin, alpha | Inexa |
| 1369133_a_at | potassium voltage gated channel, Shaw-related subfamily, | Kcnc3 |
| | member 3 | |
| 1390615_at | karyopherin alpha 1 (importin alpha 5) | Kpna1 |
| 1368573_at | karyopherin (importin) beta 1 | Kpnb1 |
| 1379410_at | lin-10 protein homolog | Lin10 |
| 1367865_at | limkain b1 | Lkap |
| 1380233_x_at | dopamine responsive protein | LOC171057 |
| 1371020_at | RIM binding protein 2 | LOC266780 |
| 1370442_at | thymosin beta-like protein | LOC286978 |
| 1367984_at | CTD-binding SR-like rA1 | LOC56081 |
| 1368664_at | dnaj-like protein | LOC56764 |
| 1395920_at | similar to RIKEN cDNA 2310036D22 | MGC94135 |
| 1372587_at | endomucin | MGC94175 |
| 1370831_at | monoglyceride lipase | Mgll |
| 1382186_a_at | similar to RIKEN cDNA 2610029K21 | na |
| 1377834_at | similar to enhancer of polycomb homolog 1 isoform 2; | na |
| | enhancer of polycomb homolog 1, (Drosophila) | |
| 1382024_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1383332_at | similar to nuclear body associated kinase 1a | na |
| 1392598_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1396403_at | LOC361748 | na |
| 1382064_at | similar to 4933407C03Rik protein | na |
| 1385235_at | similar to hypothetical protein | na |
| 1393881_at | similar to N-terminal aceyltransferase 1 | na |
| 1374874_at | similar to RIKEN cDNA 5730509K17 gene | na |
| 1377095_at | similar to ataxin 2-binding protein 1 isoform 1; | na |
| | hexaribonucleotide binding protein 1 | |
| 1375650_at | similar to bromodomain-containing protein BRD4 short variant | na |
| 1377457_a_at | similar to gp250 precursor | na |
| 1371654_at | similar to RIKEN cDNA 2510039O18 | na |
| 1393933_at | similar to gp250 precursor | na |
| 1386031_at | similar to RIKEN cDNA 4930557O20 | na |
| 1386817_at | similar to SERINE/THREONINE-PROTEIN KINASE | na |
| | PCTAIRE-3 | |
| 1371836_at | similar to Rab5c protein | na |
| 1393795_at | similar to mKIAA0569 protein | na |
| 1380329_at | similar to transmembrane protein TMP10 | na |
| 1385491_at | similar to KIAA1183 protein | na |
| 1372438_at | similar to Nit protein 2 | na |
| 1378904_at | similar to hypothetical protein A930013N06 | na |
| 1398420_at | similar to E3 ubiquitin ligase SMURF2 | na |
| 1383013_at | similar to Krueppel-like factor 13 (Transcription factor BTEB3) | na |
| | (Basic transcription element binding protein 3) (BTE-binding | |
| | protein 3) (RANTES factor of late activated T lymphocytes-1) | |
| | (RFLAT-1) (Transcription factor NSLP1) (Novel Spl-like zinc | |
| | finger) | |
| 1397959_at | similar to RIKEN cDNA D130059P03 gene | na |
| 1391578_at | similar to modulator of estrogen induced transcription | na |
| 1383466_at | similar to dJ862K6.2.2 (splicing factor, arginine/serine-rich 6 | na |
| | (SRP55-2) (isoform 2)) | |
| 1384125_at | similar to MLL5 | na |
| 1394412_at | similar to chromosome 11 open reading frame 25 | na |
| 1392422_at | similar to Disrupted in bipolar disorder 1 homolog | na |
| 1390488_a_at | similar to Serine/threonine kinase 38 | na |
| 1380172_at | similar to mKIAA0531 protein | na |
| 1394243_at | similar to spermine synthase | na |
| 1381542_at | similar to RIKEN cDNA 1300013G12 | na |
| 1391618_at | similar to KAP3A | na |
| 1390048_at | similar to splicing coactivator subunit SRm300; RNA binding | na |
| | protein; AT-rich element binding factor | |
| 1379765_at | (similar to nemo-like kinase, similar to nemo-like kinase; likely | na |
| | ortholog of mouse nemo like kinase) | |
| 1383615_a_at | similar to E3 ligase for inhibin receptor | na |
| 1398065_at | similar to hypothetical protein FLJ10159 | na |
| 1392576_at | similar to C33A12.3-like | na |
| 1385967_at | similar to Interleukin enhancer-binding factor 1 (Cellular | na |
| | transcription factor ILF-1) | |
| 1391075_at | similar to regulator of G-protein signaling Z2 | na |
| 1376523_at | similar to retinoblastoma-binding protein 1 isoform I; | na |
| | retinoblastoma-binding protein 1 | |
| 1391523_at | similar to RIKEN cDNA 4631422C05; similar to alanine | na |
| | aminotransferase 2; glutamic-pyruvate transaminase 2 | |
| 1394155_at | similar to RIKEN cDNA 1500011D06 | na |
| 1373737_at | similar to hypothetical protein | na |
| 1377016_at | similar to RIKEN cDNA 5730592L21 | na |
| 1383827_at | similar to tousled-like kinase 1 | na |
| 1395699_at | similar to sudD, suppressor of bimD6 homolog | na |
| 1397513_at | hypothetical LOC294883 | na |
| 1376108_at | LOC362299 | na |
| 1370946_at | nuclear factor I/X | Nfix |
| 1370687_a_at | neurotrophic tyrosine kinase, receptor, type 2 | Ntrk2 |
| 1391625_at | neural Wiskott-Aldrich syndrome protein | N-WASP |
| 1368958_at | protein kinase C and casein kinase substrate in neurons 1 | Pacsin1 |
| 1368438_at | phosphodiesterase 10A | Pde10a |
| 1370669_a_at | phosphodiesterase 10A | Pde10a |
| 1370052_at | 3-phosphoinositide dependent protein kinase-1 | Pdpk1 |
| 1382303_at | phosphatase and actin regulator 1 | Phactr1 |
| 1374593_at | protein kinase C, epsilon | Prkce |
| 1367884_at | RAB14, member RAS oncogene family | Rab14 |
| 1370758_at | RAB15, member RAS onocogene family | Rab15 |
| 1391347_at | RAB8B, member RAS oncogene family | Rab8b |
| 1369780_at | RAS protein-specific guanine nucleotide-releasing factor 2 | Rasgrf2 |
| 1368564_at | solute carrier family 17 (sodium-dependent inorganic phosphate | Slc17a6 |
| | cotransporter), member 6 | |
| 1369756_a_at | solute carrier family 4, member 4 | Slc4a4 |
| 1387280_a_at | tumor-associated protein 1 | Slc7a5 |
| 1385074_at | SWI/SNF related, matrix associated, actin dependent regulator | Smarca2 |
| | of chromatin, subfamily a, member 2 | |
| 1383520_at | serine-arginine-rich splicing regulatory protein 86 | Srrp86 |
| 1368842_at | transcription factor 4 | Tcf4 |
| 1370691_a_at | thyroid hormone receptor alpha | Thra |
| 1369164_a_at | transient receptor potential cation channel, subfamily C, | Trpc4 |
| | member 4 | |
| 1396170_at | WW domain binding protein 4 (formin binding protein 21) | Wbp4 |
| 1368641_at | wingless-type MMTV integration site family, member 4 | Wnt4 |

Genes and gene fragments identified as changing significantly in animals 6 hours following water maze training relative to controls are provided in Table 7 below.

TABLE 7

| Fragment Name | Gene Name | Gene Symbol |
|---|---|---|
| 1383096_at | (amyloid beta (A4) precursor-like protein 2, sperm membrane | (Aplp2, LOC64312) |
| | protein (YWK-II)) | |
| 1399162_a_at | (damage-specific DNA binding protein 1, similar to DNA | (Ddb1, na) |
| | damage binding protein 1 (Damage-specific DNA binding | |
| | protein 1) (DDB p127 subunit) (DDBa) (UV-damaged DNA- | |
| | binding protein 1) (UV-DDB 1) (Xeroderma pigmentosum | |
| | group E complementing protein) (XPCe) (X | |
| 1368411_a_at | (hypothetical gene supported by NM_013066, microtubule- | (Mtap2, na) |
| | associated protein 2) | |
| 1386961_at | (hypothetical gene supported by NM_031715, | (Pfkm, na) |
| | phosphofructokinase, muscle) | |
| 1371776_at | (hypothetical gene supported by NM_013005, | (Pik3r1, na) |
| | phosphatidylinositol 3-kinase, regulatory subunit, polypeptide | |
| | 1) | |
| 1387538_at | acetyl-coenzyme A carboxylase | Acac |
| 1387302_at | adenylate cyclase activating polypeptide 1 receptor 1 | Adcyap1r1 |
| 1369402_at | activity-dependent neuroprotective protein | Adnp |
| 1371132_a_at | ankyrin 3 (G) | Ank3 |
| 1380533_at | amyloid beta (A4) precursor protein | App |
| 1387447_at | ADP-ribosylation factor 3 | Arf3 |
| 1393596_at | alpha thalassemia/mental retardation syndrome X-linked | Atrx |
| | (RAD54 homolog, S. cerevisiae) | |
| 1369256_at | beta-site APP cleaving enzyme | Bace |
| 1387882_at | basic transcription element binding protein 1 | Bteb1 |
| 1388187_at | calcium/calmodulin-dependent protein kinase II alpha subunit | Camk2a |
| 1369538_at | cyclin-dependent kinase 5, regulatory subunit 1 (p35) | Cdk5r |
| 1387170_at | casein kinase II, alpha 1 polypeptide | Csnk2a1 |
| 1370123_a_at | cortactin isoform B | Cttn |
| 1391406_at | degenerative spermatocyte homolog (Drosophila) | Degs |
| 1387210_at | discs, large homolog 4 (Drosophila) | Dlgh4 |
| 1369813_at | cysteine string protein | Dnajc5 |
| 1369540_at | EF hand calcium binding protein 1 | Efcbp1 |
| 1368536_at | ectonucleotide pyrophosphatase/phosphodiesterase 2 | Enpp2 |
| 1369703_at | endothelial PAS domain protein 1 | Epas1 |
| 1374320_at | coagulation factor 5 | F5 |
| 1387606_at | fibroblast growth factor 2 | Fgf2 |
| 1383019_at | FMS-like tyrosine kinase 1 | Flt1 |
| 1387685_at | frequenin homolog (Drosophila) | Freq |
| 1370760_a_at | glutamate decarboxylase 1 | Gad1 |
| 1369462_at | glutamate decarboxylase 2 | Gad2 |
| 1387095_at | guanine nucleotide binding protein, alpha z subunit | Gnaz |
| 1369036_at | glutamate receptor, ionotropic, kainate 2 | Grik2 |
| 1370344_at | heat shock 70 kDa protein 4 | Hspa4 |
| 1369860_a_at | 5-hydroxytryptamine (serotonin) receptor 2C | Htr2c |
| 1387467_at | potassium inwardly-rectifying channel, subfamily J, member 10 | Kcnj10 |
| 1391906_at | kinesin family member 1B | Kif1b |
| 1382787_at | kinesin family member 5A | Kif5a |
| 1388748_at | lysosomal-associated protein transmembrane 4 alpha | Laptm4a |
| 1392254_at | microtubule-associated protein 4 | LOC367171 |
| 1367797_at | multiple endocrine neoplasia 1 | Men1 |
| 1382024_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1397675_at | similar to Wbscr1 | na |
| 1378666_at | similar to KIAA1424 protein | na |
| 1391043_at | similar to C15orf16 protein | na |
| 1376569_at | similar to Kruppel-like factor 2 (Lung kruppel-like factor) | na |
| 1382561_at | similar to 4921517L17Rik protein | na |
| 1397405_at | similar to Probable RNA-dependent helicase p72 (DEAD-box | na |
| | protein p72) (DEAD-box protein 17) | |
| 1381386_at | similar to ring finger protein 10 | na |
| 1398062_at | similar to hypothetical protein AL133206 | na |
| 1378740_at | similar to Ras GTPase-activating protein nGAP (RAS protein | na |
| | activator like 1) | |
| 1392178_at | similar to neuronal transmembrane protein Slitrk1 | na |
| 1397484_at | similar to Cytosolic acyl coenzyme A thioester hydrolase, | na |
| | inducible (Long chain acyl-CoA thioester hydrolase) (Long | |
| | chain acyl-CoA hydrolase) (CTE-I) (LACH2) (ACH2) | |
| 1372876_at | similar to Selenide, water dikinase 2 (Selenophosphate | na |
| | synthetase 2) (Selenium donor protein 2) | |
| 1383925_at | similar to D4Wsu114e protein | na |
| 1377434_at | similar to membrane-type frizzled-related protein | na |
| 1378572_at | similar to TOLLIP protein | na |
| 1377580_at | similar to KIAA0738 gene product | na |
| 1391618_at | similar to KAP3A | na |
| 1395791_at | similar to mKIAA1402 protein | na |
| 1383592_at | similar to RIKEN cDNA 1200015K23 | na |
| 1382939_at | similar to nuclear pore complex-associated intranuclear coiled- | na |
| | coil protein TPR | |
| 1398065_at | similar to hypothetical protein FLJ10159 | na |
| 1374139_at | similar to cerebellar degeneration-related 2 | na |
| 1375193_at | similar to Kunitz-type protease inhibitor 1 precursor | na |
| | (Hepatocyte growth factor activator inhibitor type 1) (HAI-1) | |
| 1379911_at | similar to Dapk1 protein | na |
| 1389216_at | similar to Ubiquitin carboxyl-terminal hydrolase 24 (Ubiquitin | na |
| | thiolesterase 24) (Ubiquitin-specific processing protease 24) | |
| | (Deubiquitinating enzyme 24) | |
| 1375528_at | similar to intracellular membrane-associated calcium- | na |
| | independent phospholipase A2 gamma | |
| 1398654_at | similar to WDR9 protein, form A | na |
| 1384773_at | similar to dendritic cell-derived ubiquitin-like protein | na |
| 1382290_at | similar to CG9643-PA | na |
| 1392864_at | similar to p190-B | na |
| 1383082_at | similar to PLU1 | na |
| 1395719_at | similar to RIKEN cDNA 2300002G02 | na |
| 1379485_at | similar to eukaryotic translation initiation factor 3, subunit 10 | na |
| | theta, 150/170 kDa; eukaryotic translation initiation factor 3, | |
| | subunit 10 (theta, 170 kD); Eukaryotic translation initiation | |
| | factor 3, subunit 10, 170 kD; eukaryotic translation initia . . . | |
| 1398649_at | similar to neuronal transmembrane protein Slitrk3 | na |
| 1381063_at | similar to adenylyl cyclase type 9 | na |
| 1378309_at | similar to Ubiquitin carboxyl-terminal hydrolase 20 (Ubiquitin | na |
| | thiolesterase 20) (Ubiquitin-specific processing protease 20) | |
| | (Deubiquitinating enzyme 20) | |
| 1395379_at | similar to U2af1-rs2 | na |
| 1382385_at | similar to proteasome 26S ATPase subunit 6 | na |
| 1393841_at | similar to hypothetical protein FLJ31810 | na |
| 1384328_at | similar to TOM1 | na |
| 1375494_a_at | neuroligin 3 | Nlgn3 |
| 1370063_at | nuclear receptor subfamily 2, group F, member 2 | Nr2f2 |
| 1387639_at | neural visinin-like Ca2+-binding protein type 2 | Nvjp2 |
| 1392890_at | platelet-activating factor acetylhydrolase, isoform Ib, alpha | Pafah1b1 |
| | subunit 45 kDa | |
| 1382943_at | protocadherin gamma subfamily C, 3 | Pcdhgc3 |
| 1369172_at | phosphodiesterase 1A, calmodulin-dependent | Pde1a |
| 1369882_at | prodynorphin | Pdyn |
| 1369794_a_at | 6-phosphofructo-2-kinase/fructose-2,6-biphosphatase 3 | Pfkfb3 |
| 1368267_at | protein-O-mannosyltransferase 1 | Pomt1 |
| 1368213_at | P450 (cytochrome) oxidoreductase | Por |
| 1370552_at | protein phosphatase 1F (PP2C domain containing) | Ppm1f |
| 1369297_at | protein phosphatase 2 (formerly 2A), regulatory subunit B (PR | Ppp2r2c |
| | 52), gamma isoform | |
| 1370488_a_at | protein tyrosine phosphatase, receptor type, D | Ptprd |
| 1370758_at | RAB15, member RAS onocogene family | Rab15 |
| 1370780_at | RAB31, member RAS oncogene family | Rab31 |
| 1388071_x_at | RT1 class Ib, locus Aw2 | RT1-Aw2 |
| 1387707_at | solute carrier family 2 (facilitated glucose transporter), member 3 | Slc2a3 |
| 1368082_at | solute carrier family 4, member 2 | Slc4a2 |
| 1387280_a_at | tumor-associated protein 1 | Slc7a5 |
| 1368606_at | solute carrier organic anion transporter family, member 1A2 | Slco1a2 |
| 1379281_at | uterine sensitization-associated gene 1 protein | Sostdc1 |
| 1369718_at | TRAP-complex gamma subunit | Ssr3 |
| 1369919_at | thyrotroph embryonic factor | Tef |
| 1367598_at | transthyretin | Ttr |

Genes and gene fragments identified as changing significantly in animals 12 hours following water maze training relative to controls are provided in Table 8 below.

TABLE 8

| Fragment Name | Gene Name | Gene Symbol |
|---|---|---|
| 1384239_at | (ribosomal protein L31, similar to ribosomal protein L31) | (Rpl31, na) |
| 1370812_at | Bcl2-like 1 | Bcl2l1 |
| 1387428_at | calcium channel, voltage-dependent, beta 1 subunit | Cacnb1 |
| 1370185_at | contactin associated protein 1 | Cntnap1 |
| 1370026_at | crystallin, alpha B | Cryab |
| 1382574_at | casein kinase 1, gamma 3 | Csnk1g3 |
| 1391406_at | degenerative spermatocyte homolog (Drosophila) | Degs |
| 1369124_at | 5-hydroxytryptamine (serotonin) receptor 2A | Htr2a |
| 1367571_a_at | insulin-like growth factor 2 | Igf2 |
| 1369793_a_at | melanoma cell adhesion molecule | Mcam |
| 1367682_at | midkine | Mdk |
| 1390165_at | similar to RIKEN cDNA 5830404H04 | MGC72630 |
| 1385101_a_at | Unknown (protein for MGC: 73017) | MGC73017 |
| 1385471_at | similar to Probable ATP-dependent RNA helicase DDX27 | na |
| | (DEAD-box protein 27) | |
| 1372577_at | similar to actin related protein ⅔ complex, subunit 4; actin | na |
| | related protein ⅔ complex, subunit 4 (20 kDa) | |
| 1389980_at | similar to Protein HSPC163 | na |
| 1389043_at | similar to aarF domain containing kinase 4 | na |
| 1393918_at | similar to mitochondrial protein of bilaterial origin like | na |
| | (2H598C) | |
| 1371573_at | similar to large subunit ribosomal protein L36a | na |
| 1371313_at | similar to 60S ribosomal protein L23a | na |
| 1389077_at | similar to CGI-141 protein; EST AA407874 | na |
| 1388578_at | similar to membrane targeting translocation | na |
| | system protein like (2A562) | |
| 1374733_at | similar to 4632415H16Rik protein | na |
| 1385835_at | similar to tumor endothelial marker 7 precursor | na |
| 1383250_at | similar to KIAA0266 gene product | na |
| 1382385_at | similar to proteasome 26S ATPase subunit 6 | na |
| 1382752_at | similar to 53BP1 protein | na |
| 1383292_at | similar to inner centromere protein-B | na |
| 1370687_a_at | neurotrophic tyrosine kinase, receptor, type 2 | Ntrk2 |
| 1369150_at | pyruvate dehydrogenase kinase, isoenzyme 4 | Pdk4 |
| 1377061_at | RhoGAP involved in beta-catenin-N-cadherin and NMDA | RICS |
| | receptor signaling | |
| 1387098_at | RNA polymerase 1-4 | Rpo1-4 |
| 1373914_at | stem cell growth factor | Scgf |
| 1368082_at | solute carrier family 4, member 2 | Slc4a2 |
| 1369020_at | solute carrier family 5 (sodium iodide symporter), member 5 | Slc5a5 |
| 1368186_a_at | spleen tyrosine kinase | Syk |
| 1373922_at | TPR domain, ankyrin-repeat and coiled-coil-containing | Tanc |

Genes and gene fragments identified as changing significantly in animals 24 hours following water maze training relative to controls are provided in Table 9 below.

TABLE 9

| Fragment Name | Gene Name | Gene Symbol |
|---|---|---|
| 1367617_at | (aldolase A, similar to Fructose-bisphosphate aldolase A | (Aldoa, na) |
| | (Muscle-type aldolase)) | |
| 1383096_at | (amyloid beta (A4) precursor-like protein 2, sperm membrane | (Aplp2, LOC64312) |
| | protein (YWK-II)) | |
| 1369937_at | (calmodulin 1, hypothetical gene supported by NM_031969; | (Calm1, na) |
| | AF178845) | |
| 1369936_at | (calmodulin 1, hypothetical gene supported by NM_031969; | (Calm1, na) |
| | AF178845) | |
| 1387772_at | (calmodulin 1, hypothetical gene supported by NM_031969; | (Calm1, na) |
| | AF178845) | |
| 1373470_at | (beta-catenin, similar to beta-catenin; catenin beta) | (Catnb, na) |
| 1386921_at | (carboxypeptidase E, similar to Carboxypeptidase H precursor | (Cpe, na) |
| | (CPH) (Carboxypeptidase E) (CPE) (Enkephalin convertase) | |
| | (Prohormone processing carboxypeptidase)) | |
| 1369939_at | (cytochrome c, somatic, similar to Cytochrome c, somatic) | (Cycs, na) |
| 1399162_a_at | (damage-specific DNA binding protein 1, similar to DNA | (Ddb1, na) |
| | damage binding protein 1 (Damage-specific DNA binding | |
| | protein 1) (DDB p127 subunit) (DDBa) (UV-damaged DNA- | |
| | binding protein 1) (UV-DDB 1) (Xeroderma pigmentosum | |
| | group E complementing protein) (XPCe) (X | |
| 1388110_at | (eukaryotic translation elongation factor 1 alpha 1, eukaryotic | (Eef1a1, Eef1a2, na) |
| | translation elongation factor 1 alpha 2, hypothetical | |
| | LOC293924, similar to Elongation factor 1-alpha 1 (EF-1- | |
| | alpha-1) (Elongation factor 1 A-1) (eEF1A-1) (Elongation | |
| | factor Tu) (EF-Tu)) | |
| 1370109_s_at | (eukaryotic translation elongation factor 1 alpha 1, eukaryotic | (Eef1a1, Eef1a2, na) |
| | translation elongation factor 1 alpha 2, hypothetical | |
| | LOC293924, similar to Elongation factor 1-alpha 1 (EF-1- | |
| | alpha-1) (Elongation factor 1 A-1) (eEF1A-1) (Elongation | |
| | factor Tu) (EF-Tu)) | |
| 1398829_at | (FK506 binding protein 2, FK506-binding protein 1a) | (Fkbp1a, Fkbp2) |
| 1398828_at | (FK506 binding protein 2, FK506-binding protein 1a) | (Fkbp1a, Fkbp2) |
| 1367565_a_at | (ferritin, heavy polypeptide 1, similar to ferritin, heavy | (Fth1, na) |
| | polypeptide 1; Ferritin subunit H) | |
| 1367557_s_at | (glyceraldehyde-3-phosphate dehydrogenase, similar to | (Gapd, na) |
| | glyceraldehyde-3-phosphate dehydrogenase, similar to | |
| | glyceraldehyde-3-phosphate dehydrogenase (phosphorylating) | |
| | (EC 1.2.1.12) - mouse, similar to glyceraldehyde-3-phosphate- | |
| | dehydrogenase (EC 1.2.1.12) | |
| 1368353_at | (glial fibrillary acidic protein, hypothetical gene supported by | (Gfap, na) |
| | L27219; NM_017009) | |
| 1370240_x_at | (hemoglobin alpha, adult chain 1, similar to hemoglobin alpha | (Hba-a1, na) |
| | chain) | |
| 1370239_at | (hemoglobin alpha, adult chain 1, similar to hemoglobin alpha | (Hba-a1, na) |
| | chain) | |
| 1371102_x_at | (hemoglobin beta chain complex, similar to Hemoglobin beta | (Hbb, na) |
| | chain, minor-form) | |
| 1398240_at | (heat shock protein 8, similar to Heat shock cognate 71 kDa | (Hspa8, na) |
| | protein) | |
| 1381677_at | (RNA binding protein HuB, hypothetical gene supported by | (Hub, na) |
| | NM_173309) | |
| 1395357_at | (microtubule-associated protein 1b, similar to Microtubule- | (Map1b, na) |
| | associated protein 1B (MAP 1B) (Neuraxin)) | |
| 1373363_at | (microtubule-associated protein 1b, similar to Microtubule- | (Map1b, na) |
| | associated protein 1B (MAP 1B) (Neuraxin)) | |
| 1387071_a_at | (hypothetical gene supported by NM_017212, microtubule- | (Mapt, na) |
| | associated protein tau) | |
| 1370948_a_at | (myristoylated alanine rich protein kinase C substrate, similar to | (Marcks, na) |
| | Myristoylated alanine-rich C-kinase substrate (MARCKS)) | |
| 1373432_at | (myristoylated alanine rich protein kinase C substrate, similar to | (Marcks, na) |
| | Myristoylated alanine-rich C-kinase substrate (MARCKS)) | |
| 1368151_at | (hypothetical LOC293831, matrin 3) | (Matr3, na) |
| 1388372_at | similar to 60S RIBOSOMAL PROTEIN L35 | (MGC72958, na) |
| 1370434_a_at | (hypothetical gene supported by NM_012720, myelin- | (Mobp, na) |
| | associated oligodendrocytic basic protein) | |
| 1388152_at | (hypothetical gene supported by NM_013066, microtubule- | (Mtap2, na) |
| | associated protein 2) | |
| 1370517_at | (hypothetical gene supported by NM_153735, neuronal | (Nptx1, Ntsr2, na) |
| | pentraxin 1, neurotensin receptor 2) | |
| 1370941_at | (platelet derived growth factor receptor, alpha polypeptide, | (Pdgfra, na) |
| | similar to Alpha platelet-derived growth factor receptor | |
| | precursor (PDGF-R-alpha)) | |
| 1388311_at | (T-cell activation protein, similar to Ab2-450) | (Pgr1, na) |
| 1388332_at | (ras-related C3 botulinum toxin substrate 1 (rho family, small | (Rac1, na) |
| | GTP binding protein Rac1), similar to ras-related C3 botulinum | |
| | toxin substrate 1 isoform Rac1b; rho family, small GTP binding | |
| | protein Rac1) | |
| 1367625_at | (hypothetical LOC291740, ribosomal protein L10, similar to | (Rpl10, na) |
| | 60S ribosomal protein L10 (QM protein homolog)) | |
| 1398871_at | (ribosomal protein L17, similar to 60S RIBOSOMAL | (Rpl17, na) |
| | PROTEIN L17 (L23) (AMINO ACID STARVATION- | |
| | INDUCED PROTEIN) (ASI), similar to Heph protein) | |
| 1367610_at | (ribosomal protein L19, similar to 60S ribosomal protein L19) | (Rpl19, na) |
| 1398830_at | (ribosomal protein L28, similar to ribosomal protein L28) | (Rpl28, na) |
| 1371300_at | (ribosomal protein L3, similar to 60S RIBOSOMAL PROTEIN | (Rpl3, na) |
| | L3 (L4)) | |
| 1367634_at | (ribosomal protein L31, similar to ribosomal protein L31) | (Rpl31, na) |
| 1398760_at | (ribosomal protein L35a, similar to ribosomal protein L35a) | (Rpl35a, na) |
| 1398789_at | (ribosomal protein L37, similar to ribosomal protein L37) | (Rpl37, na) |
| 1398761_at | (ribosomal protein L5, similar to 60S RIBOSOMAL PROTEIN | (Rpl5, na) |
| | L5, similar to ribosomal protein L5) | |
| 1371301_at | (ribosomal protein L9, similar to 60S RIBOSOMAL PROTEIN | (Rpl9, na) |
| | L9, similar to ribosomal protein L9) | |
| 1371307_at | (ribosomal protein, large, P1, similar to 60S ACIDIC | (Rplp1, na) |
| | RIBOSOMAL PROTEIN P1) | |
| 1371340_at | (ribosomal protein, large P2, similar to 60S ACIDIC | (Rplp2, na) |
| | RIBOSOMAL PROTEIN P2) | |
| 1372845_at | (ribonuclease P 21 kDa subunit (human), tripartite motif protein | (Rpp21, Trim39) |
| | 39) | |
| 1367630_at | (ribosomal protein S11, similar to 40S ribosomal protein S11) | (Rps11, na) |
| 1371318_at | (ribosomal protein S16, similar to 40S ribosomal protein S16) | (Rps16, na) |
| 1367645_at | (ribosomal protein S17, similar to 40S RIBOSOMAL | (Rps17, na) |
| | PROTEIN S17) | |
| 1388296_at | (ribosomal protein S18, similar to ribosomal protein S18, | (Rps18, na) |
| | similar to ribosomal protein S18, cytosolic [validated] - rat) | |
| 1375219_a_at | (ribosomal protein S2, similar to 40S ribosomal protein S2) | (Rps2, na) |
| 1370242_at | (ribosomal protein S23, similar to ribosomal protein S23) | (Rps23, na) |
| 1367685_at | (ribosomal protein S27a, similar to ribosomal protein S27a) | (Rps27a, na) |
| 1398751_at | (hypothetical LOC298661, ribosomal protein S7) | (Rps7, na) |
| 1375170_at | (S100 calcium binding protein A11 (calizzarin), similar to | (S100a11, na) |
| | endothelial monocyte-activating polypeptide) | |
| 1368098_a_at | (SNRPN upstream reading frame, similar to small nuclear | (Snrpn, Snurf, na) |
| | ribonucleoprotein-associated protein N - rat, small nuclear | |
| | ribonucleoprotein N) | |
| 1387195_at | (similar to suppression of tumorigenicity 14; suppression of | (St14, na) |
| | tumorigenicity 14 (colon carcinoma, matriptase, epithin), | |
| | suppression of tumorigenicity 14) | |
| 1390196_at | (similar to Synapsin I, synapsin I) | (Syn1, na) |
| 1369251_a_at | (similar to Synapsin I, synapsin I) | (Syn1, na) |
| 1367583_at | (similar to Translationally controlled tumor protein (TCTP) | (Tpt1, na) |
| | (p23) (21 kDa polypeptide) (p21) (Lens epithelial protein), | |
| | tumor protein, translationally-controlled 1) | |
| 1367579_a_at | (hypothetical gene supported by NM_022298; NM_022298; | (Tuba1, na) |
| | V01227, tubulin, alpha 1) | |
| 1387862_at | (similar to 14-3-3 PROTEIN TAU (14-3-3 PROTEIN THETA), | (Ywhaq, na) |
| | similar to 14-3-3 protein tau (14-3-3 protein theta) (14-3-3 | |
| | protein T-cell) (HS1 protein), tyrosine 3- | |
| | monooxygenase/tryptophan 5-monooxygenase activation | |
| | protein, theta polypeptide) | |
| 1398836_s_at | actin, beta | Actb |
| 1398835_at | actin, beta | Actb |
| 1378566_at | adenylate cyclase 1 | Adcy1 |
| 1387861_at | amino-terminal enhancer of split | Aes |
| 1386998_at | aldolase C, fructose-biphosphate | Aldoc |
| 1367775_at | alpha-methylacyl-CoA racemase | Amacr |
| 1370862_at | apolipoprotein E | Apoe |
| 1371571_at | amyloid beta (A4) precursor protein | App |
| 1388997_at | ADP-ribosylation factor 3 | Arf3 |
| 1370611_at | aryl hydrocarbon receptor nuclear translocator 2 | Arnt2 |
| 1386911_at | ATPase, Na+K+ transporting, alpha 2 | Atp1a2 |
| 1367814_at | ATPase, Na+/K+ transporting, beta 1 polypeptide | Atp1b1 |
| 1386937_at | ATPase, Na+/K+ transporting, beta 1 polypeptide | Atp1b1 |
| 1370378_at | mitochondrial H+-ATP synthase alpha subunit | Atp5a1 |
| 1370275_at | ATP synthase, H+ transporting, mitochondrial F1 complex, beta | Atp5b |
| | polypeptide | |
| 1367620_at | ATP synthase, H+ transporting, mitochondrial F0 complex, | Atp5g3 |
| | subunit c (subunit 9) isoform 3 | |
| 1371402_at | ATPase, H+ transporting, lysosomal (vacuolar proton pump), | Atp6b2 |
| | beta 56/58 kDa, isoform 2 | |
| 1398755_at | ATPase, H+ transporting, lysosomal (vacuolar proton pump) 16 kDa | Atp6l |
| 1387317_at | arginine vasopressin | Avp |
| 1387234_at | alpha-2-glycoprotein 1, zinc | Azgp1 |
| 1398350_at | brain abundant, membrane attached signal protein 1 | Basp1 |
| 1370812_at | Bcl2-like 1 | Bcl2l1 |
| 1370246_at | calmodulin 2 | Calm2 |
| 1370873_at | calmodulin 3 | Calm3 |
| 1388187_at | calcium/calmodulin-dependent protein kinase II alpha subunit | Camk2a |
| 1398827_at | CD 81 antigen | Cd81 |
| 1370184_at | cofilin 1 | Cfl1 |
| 1368287_at | chimerin (chimaerin) 1 | Chn1 |
| 1367784_a_at | clusterin | Clu |
| 1367607_at | cytochrome c oxidase subunit IV isoform 1 | Cox4i1 |
| 1367757_at | cytochrome oxidase subunit VIc | Cox6c |
| 1371387_at | cytochrome c oxidase subunit VIIb | Cox7b |
| 1388113_at | cytochrome c oxidase, subunit VIIIa | Cox8a |
| 1398357_at | complexin 1 | Cplx1 |
| 1368584_a_at | complexin 2 | Cplx2 |
| 1370596_a_at | SH3 domain binding protein CR16 | Cr16 |
| 1369908_at | corticotropin releasing hormone binding protein | Crhbp |
| 1369912_at | v-crk sarcoma virus CT10 oncogene homolog (avian) | Crk |
| 1370855_at | cystatin C | Cst3 |
| 1384392_at | cytochrome P450, family 26, subfamily b, polypeptide 1 | Cyp26b1 |
| 1370507_at | disks large-associated protein 4 | Dap4 |
| 1388177_at | dendrin | Ddn |
| 1375462_at | discoidin domain receptor family, member 1 | Ddr1 |
| 1391406_at | degenerative spermatocyte homolog (Drosophila) | Degs |
| 1368292_at | dynamin 1 | Dnm1 |
| 1376183_at | dipeptidylpeptidase 6 | Dpp6 |
| 1371694_at | dihydropyrimidinase-like 2 | Dpysl2 |
| 1370183_at | dual-specificity tyrosine-(Y)-phosphorylation regulated kinase | Dyrk1a |
| | 1A | |
| 1370003_at | eukaryotic translation elongation factor 2 | Eef2 |
| 1388666_at | ectodermal-neural cortex 1 | Enc1 |
| 1368536_at | ectonucleotide pyrophosphatase/phosphodiesterase 2 | Enpp2 |
| 1369703_at | endothelial PAS domain protein 1 | Epas1 |
| 1388358_at | electron-transfer-flavoprotein, beta polypeptide | Etfb |
| 1374320_at | coagulation factor 5 | F5 |
| 1368114_at | fibroblast growth factor 13 | Fgf13 |
| 1367700_at | fibromodulin | Fmod |
| 1387889_at | folate receptor 1 (adult) | Folr1 |
| 1384723_at | alpha3-fucosyltransferase 11 | Fut11 |
| 1367632_at | glutamine synthetase 1 | Glu1 |
| 1369897_s_at | GNAS complex locus | Gnas |
| 1369410_at | golgi SNAP receptor complex member 1 | Gosr1 |
| 1386955_at | glycoprotein Ib (platelet), beta polypeptide | Gp1bb |
| 1387746_at | gustatory receptor 43 | Gust43 |
| 1388608_x_at | hemoglobin alpha, adult chain 1 | Hba-a1 |
| 1387387_at | hippocalcin | Hpca |
| 1369562_at | neural visinin-like Ca2+-binding protein type 3 | Hpcal1 |
| 1384295_at | heat shock protein hsp70-related protein | Hspa14 |
| 1370344_at | heat shock 70 kDa protein 4 | Hspa4 |
| 1388850_at | heat shock protein 1, alpha | Hspca |
| 1372701_at | heat shock protein 1, alpha | Hspca |
| 1375335_at | heat shock 90 kDa protein 1, beta | Hspcb |
| 1375336_at | heat shock 90 kDa protein 1, beta | Hspcb |
| 1369809_at | 5-hydroxytryptamine (serotonin) receptor 1A | Htr1a |
| 1367787_at | islet cell autoantigen 1, 69 kDa | Ica1 |
| 1368123_at | insulin-like growth factor 1 receptor | Igf1r |
| 1367571_a_at | insulin-like growth factor 2 | Igf2 |
| 1369359_at | interleukin 9 receptor | Il9r |
| 1387061_at | junction plakoglobin | Jup |
| 1387467_at | potassium inwardly-rectifying channel, subfamily J, member 10 | Kcnj10 |
| 1388034_at | kinesin family member 1B | Kif1b |
| 1387657_at | kinesin family member 3C | Kif3c |
| 1378449_at | keratinocytes proline-rich protein | Kprp |
| 1375726_at | LIM domain only protein 7 | LMO7 |
| 1398880_at | pR-ET2 encoded oncodevelopmental protein | Loc192269 |
| 1377080_at | p75-like apoptosis-inducing death domain protein PLAIDD | LOC246143 |
| 1374307_at | CaM-kinase II inhibitor alpha | LOC287005 |
| 1387646_a_at | Max protein | Max |
| 1387341_a_at | myelin basic protein | Mbp |
| 1368810_a_at | myelin basic protein | Mbp |
| 1367653_a_at | malate dehydrogenase 1, NAD (soluble) | Mdh1 |
| 1367682_at | midkine | Mdk |
| 1398324_at | similar to 60S ribosomal protein L18a | MGC72957 |
| 1388955_at | putative eps protein | MGEPS |
| 1368302_at | homeo box, msh-like 1 | Msx1 |
| 1370124_at | metallothionein 3 | Mt3 |
| 1387732_at | transcription termination factor, mitochondrial | Mterf |
| 1393407_at | similar to F-box protein FWD2 | na |
| 1385713_at | similar to NAKAP95 | na |
| 1391224_at | similar to 4921517L17Rik protein | na |
| 1374516_at | similar to RIKEN cDNA 5830457O10 | na |
| 1380235_at | similar to heparan sulfate 2-sulfotransferase | na |
| 1389433_at | similar to McKusick-Kaufman syndrome protein; MKKS | na |
| | protein | |
| 1377855_at | similar to RIKEN cDNA 4921537D05 | na |
| 1381564_at | similar to glomulin, FKBP associated protein; FKBP-associated | na |
| | protein | |
| 1395364_at | similar to interferon alpha/beta receptor | na |
| 1382175_at | similar to Wilms tumour 1-associating protein isoform 2; | na |
| | putative pre-mRNA splicing regulator female-lethal(2D); | |
| | WTAP protein; PNAS-132; WT1-associated protein | |
| 1382561_at | similar to 4921517L17Rik protein | na |
| 1373152_at | similar to RIKEN cDNA 2310046G15 gene | na |
| 1397405_at | similar to Probable RNA-dependent helicase p72 (DEAD-box | na |
| | protein p72) (DEAD-box protein 17) | |
| 1388530_at | similar to small protein effector 1 of Cdc42 | na |
| 1371761_at | similar to ribosomal protein L34; 60S ribosomal protein L34 | na |
| 1374490_at | similar to thyroid hormone receptor interactor 12; thyroid | na |
| | receptor interacting protein 12 | |
| 1372751_at | similar to oriLyt TD-element binding protein 7 | na |
| 1371991_at | similar to RIKEN cDNA B230312A22 | na |
| 1377704_at | similar to KIAA1585 protein | na |
| 1385806_at | similar to PAP-1 | na |
| 1382122_at | similar to ets variant gene 5 | na |
| 1381654_at | similar to CG12467-PA | na |
| 1396040_at | hypothetical LOC292874 | na |
| 1373582_at | similar to CLIP-170-related protein | na |
| 1371320_at | similar to E25B protein | na |
| 1371474_at | similar to Mtch1 protein | na |
| 1375632_at | similar to tweety homolog 2 | na |
| 1371393_at | similar to calsyntenin-1 protein | na |
| 1398893_at | similar to Nedd4 WW domain-binding protein 5 | na |
| 1374401_at | similar to sorting nexin 2 | na |
| 1388310_at | (similar to Chain, Human Translation Initiation Factor Eifl, | na |
| | Nmr, 29 Structures, similar to Protein translation factor SUI1 | |
| | homolog) | |
| 1372953_at | similar to neurocalcin delta | na |
| 1377434_at | similar to membrane-type frizzled-related protein | na |
| 1376499_at | similar to MR1-interacting protein | na |
| 1371507_at | similar to 4921517L17Rik protein | na |
| 1388303_at | similar to 60S ribosomal protein L26 | na |
| 1398578_at | similar to leukocyte receptor cluster (LRC) member 8 | na |
| 1391618_at | similar to KAP3A | na |
| 1373266_at | similar to downregulated in renal cell carcinoma | na |
| 1376709_at | similar to RIKEN cDNA 4933419D20 | na |
| 1389904_at | similar to actin-related protein 2 | na |
| 1372437_at | similar to S-phase kinase-associated protein 1A isoform b; | na |
| | organ of Corti protein 2; transcription elongation factor B | |
| | (SIII), polypeptide 1-like; RNA polymerase II elongation | |
| | factor-like protein OCP2; cyclin A/CDK2-associated p19 | |
| 1379123_at | similar to Type I transmembrane receptor (seizure-related | na |
| | protein) | |
| 1371319_at | similar to E25B protein | na |
| 1390385_at | similar to D-glucuronyl C5-epimerase | na |
| 1398326_at | similar to Nur77 downstream protein 2 | na |
| 1375248_at | similar to Kruppel-like factor 2 (Lung kruppel-like factor) | na |
| 1390937_at | similar to chromosome 14 open reading frame 50 | na |
| 1391432_at | similar to CG12050-PA | na |
| 1398664_at | similar to RIKEN cDNA 9130427A09 | na |
| 1374484_at | similar to RIKEN cDNA 2610033C09 | na |
| 1391580_at | similar to Nit protein 2 | na |
| 1395616_at | similar to Ab2-008 | na |
| 1397333_at | similar to RIKEN cDNA A830094I09 gene | na |
| 1397708_at | similar to 3110009E18Rik protein | na |
| 1373881_at | similar to GDP-dissociation inhibitor | na |
| 1372847_at | similar to DC11 protein | na |
| 1389561_at | similar to RIKEN cDNA 1810021J13 | na |
| 1376904_at | similar to hypothetical protein MGC33486 | na |
| 1389695_at | similar to cDNA sequence BC004044; clone MGC: 7673 | na |
| 1372207_at | similar to hypothetical protein | na |
| 1383829_at | similar to HMG-box transcription factor BBX | na |
| 1393909_at | similar to KIAA1841 protein | na |
| 1398573_at | similar to Zinc finger protein 306 (Zinc finger protein zfp47) | na |
| | (Zf47) | |
| 1374605_at | similar to hypothetical protein MGC33214 | na |
| 1393198_at | similar to RIKEN cDNA 5330414D10 | na |
| 1382739_at | similar to Gpr19 protein | na |
| 1390604_s_at | similar to RIKEN cDNA 4930471O16 | na |
| 1373238_at | similar to SPT3-associated factor 42 | na |
| 1383337_at | similar to Hypothetical protein KIAA0182 | na |
| 1393593_at | similar to RIKEN cDNA F830029L24 | na |
| 1393291_at | similar to CG4502-PA | na |
| 1371344_at | (similar to 60S RIBOSOMAL PROTEIN L27A, similar to 60S | na |
| | ribosomal protein L27a) | |
| 1383389_at | similar to RIKEN cDNA C230075L19 gene | na |
| 1371642_at | similar to translation initiation factor eIF-4A II - mouse | na |
| 1389059_at | similar to Lyl-1 protein (Lymphoblastic leukemia derived | na |
| | sequence 1) | |
| 1371297_at | (similar to 60S ribosomal protein L7a (Surfeit locus protein 3) | na |
| | (PLA-X polypeptide), similar to Rpl7a protein) | |
| 1372249_at | similar to Ac1254 | na |
| 1397579_x_at | similar to RIKEN cDNA 2310011J03 | na |
| 1373385_at | similar to mahogunin, ring finger 1; mahoganoid | na |
| 1376245_x_at | similar to MR1-interacting protein | na |
| 1374431_at | similar to ATPase, H+ transporting, V1 subunit A, isoform 1 | na |
| 1372532_at | similar to M-RdgB2 retinal degeneration protein B subtype 2 | na |
| 1371767_at | similar to SMARCD1 protein | na |
| 1371497_at | similar to RIKEN cDNA 2610205E22 | na |
| 1385862_at | similar to RIKEN cDNA 4930438O05 | na |
| 1385073_at | similar to hypothetical protein DJ667H12.2 | na |
| 1394523_at | similar to RIKEN cDNA 1100001H23 | na |
| 1379056_at | similar to RIKEN cDNA 2810427I04; DNA segment, Chr 8, | na |
| | ERATO Doi 590, expressed | |
| 1371849_at | similar to hypothetical protein FLJ12442 | na |
| 1367956_at | neurochondrin | Ncdn |
| 1370229_at | N-myc downstream regulated 4 | Ndr4 |
| 1387121_a_at | N-myc downstream-regulated gene 2 | Ndrg2 |
| 1370946_at | nuclear factor I/X | Nfix |
| 1391455_at | conserved nuclear protein Nhn1 | Nhn1 |
| 1387099_at | natriuretic peptide receptor 2 | Npr2 |
| 1370211_at | neurogranin | Nrgn |
| 1369690_at | N-ethylmaleimide sensitive factor | Nsf |
| 1369689_at | N-ethylmaleimide sensitive factor | Nsf |
| 1370687_a_at | neurotrophic tyrosine kinase, receptor, type 2 | Ntrk2 |
| 1372241_at | ornithine decarboxylase antizyme 1 | Oaz1 |
| 1369008_a_at | olfactomedin 1 | Olfm1 |
| 1387927_a_at | olfactomedin 1 | Olfm1 |
| 1392890_at | platelet-activating factor acetylhydrolase, isoform Ib, alpha | Pafah1b1 |
| | subunit 45 kDa | |
| 1386891_at | phosphatidylethanolamine binding protein | Pbp |
| 1368956_at | protocadherin 8 | Pcdh8 |
| 1388551_at | protocadherin gamma subfamily C, 3 | Pcdhgc3 |
| 1382943_at | protocadherin gamma subfamily C, 3 | Pcdhgc3 |
| 1367835_at | proprotein convertase subtilisin/kexin type 1 inhibitor | Pcsk1n |
| 1369882_at | prodynorphin | Pdyn |
| 1369794_a_at | 6-phosphofructo-2-kinase/fructose-2,6-biphosphatase 3 | Pfkfb3 |
| 1386864_at | phosphoglycerate mutase 1 | Pgam1 |
| 1388318_at | phosphoglycerate kinase 1 | Pgk1 |
| 1367926_at | prohibitin | Phb |
| 1369976_at | dynein, cytoplasmic, light chain 1 | Pin |
| 1387112_at | proteolipid protein | Plp |
| 1387929_at | PMF32 protein | Pmf31 |
| 1398850_at | peptidylprolyl isomerase A | Ppia |
| 1368277_at | protein phosphatase 3, catalytic subunit, alpha isoform | Ppp3ca |
| 1373479_at | protein phosphatase 3, catalytic subunit, alpha isoform | Ppp3ca |
| 1372982_at | protein phospatase 3, regulatory subunit B, alpha isoform, type 1 | Ppp3r1 |
| 1374466_at | protein kinase C, epsilon | Prkce |
| 1370156_at | prion protein | Prnp |
| 1386892_at | parathymosin | Ptms |
| 1375163_at | RAB11B, member RAS oncogene family | Rab11b |
| 1392681_at | RAB2, member RAS oncogene family | Rab2 |
| 1385052_at | RAB3A interacting protein (rabin3)-like 1 | Rab3il1 |
| 1368660_at | cAMP-regulated guanine nucleotide exchange factor I (cAMP- | Rapgef3 |
| | GEFI) | |
| 1396575_at | RA and RhoGAP domain containing protein | RARhoGAP |
| 1399027_at | plysia ras-related homolog A2 | Rhoa |
| 1398885_at | ribosomal protein L23 | Rpl23 |
| 1398774_at | ribosomal protein L30 | Rpl30 |
| 1370866_at | ribosomal protein L41 | Rpl41 |
| 1368211_at | ribosomal protein S14 | Rps14 |
| 1386874_at | ribosomal protein S15 | Rps15 |
| 1398775_at | ribosomal protein S15a | Rps15a |
| 1367639_a_at | ribosomal protein S2 | Rps2 |
| 1398852_at | ribosomal protein S21 | Rps21 |
| 1369966_a_at | ribosomal protein S24 | Rps24 |
| 1387890_at | ribosomal protein S29 | Rps29 |
| 1371299_at | ribosomal protein S3 | Rps3 |
| 1367606_at | ribosomal protein S3a | Rps3a |
| 1367573_at | ribosomal protein S6 | Rps6 |
| 1388071_x_at | RT1 class Ib, locus Aw2 | RT1-Aw2 |
| 1368097_a_at | reticulon 1 | Rtn1 |
| 1386903_at | S100 protein, beta polypeptide | S100b |
| 1369709_at | spinocerebellar ataxia 1 homolog (human) | Sca1 |
| 1386889_at | stearoyl-Coenzyme A desaturase 2 | Scd2 |
| 1367668_a_at | stearoyl-Coenzyme A desaturase 2 | Scd2 |
| 1388253_at | stearoyl-Coenzyme A desaturase 2 | Scd2 |
| 1372180_at | syndecan 3 | Sdc3 |
| 1387016_a_at | stromal cell derived factor receptor 1 | Sdfr1 |
| 1370907_at | sialyltransferase 1 | Siat1 |
| 1370803_at | SNAP25 interacting protein 30 | Sip30 |
| 1371205_at | solute carrier family 14 (urea transporter), member 2 | Slc14a2 |
| 1368986_at | solute carrier family 17 (sodium-dependent inorganic phosphate | Slc17a7 |
| | cotransporter), member 7 | |
| 1368565_at | solute carrier family 1 (glial high affinity glutamate | Slc1a3 |
| | transporter), member 3 | |
| 1388112_at | solute carrier family 25 (mitochondrial adenine nucleotide | Slc25a4 |
| | translocator) member 4 | |
| 1369756_a_at | solute carrier family 4, member 4 | Slc4a4 |
| 1369715_at | solute carrier family 6 (neurotransmitter transporter, GABA), | Slc6a11 |
| | member 11 | |
| 1368606_at | solute carrier organic anion transporter family, member 1A2 | Slco1a2 |
| 1370143_at | smoothened homolog (Drosophila) | Smo |
| 1387073_at | synaptosomal-associated protein | Snap25 |
| 1367977_at | synuclein, alpha | Snca |
| 1371568_at | synuclein, beta | Sncb |
| 1379281_at | uterine sensitization-associated gene 1 protein | Sostdc1 |
| 1386865_at | SPARC-like 1 (mast9, hevin) | Sparcl1 |
| 1395014_at | spastic paraplegia 7 homolog (human) | Spg7 |
| 1383410_at | signal recognition particle 54 kDa | Srp54 |
| 1369718_at | TRAP-complex gamma subunit | Ssr3 |
| 1388811_at | synapsin II | Syn2 |
| 1373896_at | synaptotagmin 1 | Syt1 |
| 1369058_at | synaptotagmin 3 | Syt3 |
| 1390817_at | transcription factor 20 | Tcf20 |
| 1388307_at | tumor differentially expressed 1, like | Tde2 |
| 1371702_at | transmembrane 4 superfamily member 2 | Tm4sf2 |
| 1387883_a_at | thymosin beta-4 | Tmsb4x |
| 1371241_x_at | tropomyosin 1, alpha | Tpm1 |
| 1387617_at | tropomyosin 3, gamma | Tpm3 |
| 1367598_at | transthyretin | Ttr |
| 1373546_at | putative UA20 protein | Ua20 |
| 1398754_at | ubiquitin A-52 residue ribosomal protein fusion product 1 | Uba52 |
| 1386852_x_at | polyubiquitin | Ubb |
| 1370274_at | polyubiquitin | Ubb |
| 1387975_at | UDP-glucose ceramide glucosyltransferase | Ugcg |
| 1370238_at | upregulated during skeletal muscle growth 5 | Usmg5 |
| 1374394_at | WW domain binding protein 2 | Wbp2 |
| 1398800_at | tyrosine 3-monooxygenase/tryptophan 5-monooxygenase | Ywhab |
| | activation protein, beta polypeptide | |
| 1398851_at | tyrosine 3-monooxygenase/tryptophan 5-monooxygenase | Ywhae |
| | activation protein, epsilon polypeptide | |
| 1386866_at | tyrosine 3-monooxgenase/tryptophan 5-monooxgenase | Ywhag |
| | activation protein, gamma polypeptide | |
| 1367693_at | tyrosine 3-monooxygenase/tryptophan 5-monooxygenase | Ywhah |
| | activation protein, eta polypeptide | |
| 1387774_at | tyrosine 3-monooxygenase/tryptophan 5-monooxygenase | Ywhaz |
| | activation protein, zeta polypeptide | |

Genes and gene fragments identified as changing significantly in animals at time 0 following passive avoidance training relative to controls are provided in Table 10 below.

TABLE 10

| Fragment Name | Gene Name | Gene Symbol |
|---|---|---|
| 1368202_a_at | (disabled homolog 2, mitogen-responsive phosphoprotein | (Dab2, na) |
| | (Drosophila), hypothetical gene supported by NM_024159) | |
| 1371245_a_at | (hemoglobin beta chain complex, similar to Hemoglobin beta | (Hbb, na) |
| | chain, minor-form) | |
| 1370500_a_at | (hypothetical gene supported by NM_012720, myelin- | (Mobp, na) |
| | associated oligodendrocytic basic protein) | |
| 1381220_at | activin receptor IIA | Acvr2 |
| 1380533_at | amyloid beta (A4) precursor protein | App |
| 1386991_a_at | bcl-2 associated death agonist | Bad |
| 1382993_at | Bcl-2 binding component 3 | Bbc3 |
| 1370438_at | C-terminal PDZ domain ligand of neuronal nitric oxide | Capon |
| | synthase | |
| 1367940_at | chemokine orphan receptor 1 | Cmkor1 |
| 1368536_at | ectonucleotide pyrophosphatase/phosphodiesterase 2 | Enpp2 |
| 1377821_at | v-erb-b2 erythroblastic leukemia viral oncogene homolog 3 | Erbb3 |
| | (avian) | |
| 1374320_at | coagulation factor 5 | F5 |
| 1387889_at | folate receptor 1 (adult) | Folr1 |
| 1370212_at | homer, neuronal immediate early gene, 3 | Homer3 |
| 1367571_a_at | insulin-like growth factor 2 | Igf2 |
| 1367648_at | insulin-like growth factor binding protein 2 | Igfbp2 |
| 1382787_at | kinesin family member 5A | Kif5a |
| 1367682_at | midkine | Mdk |
| 1368450_at | myosin Va | Myo5a |
| 1377434_at | similar to membrane-type frizzled-related protein | na |
| 1392598_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1397959_at | similar to RIKEN cDNA D130059P03 gene | na |
| 1382024_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1383726_at | hypothetical LOC294715 | na |
| 1373790_at | similar to Aph1a-pending protein | na |
| 1371908_at | similar to NTF2-related export protein NXT1 | na |
| 1382130_at | similar to bA99E24.1.1 (protocadherin 19 (KIAA1313) protein) | na |
| 1390710_x_at | similar to gp250 precursor | na |
| 1373531_at | similar to cleavage and polyadenylation specificity factor 1 | na |
| 1380329_at | similar to transmembrane protein TMP10 | na |
| 1389482_at | similar to EST AA792894 | na |
| 1376250_at | similar to nuclear FMRP interacting protein 1 | na |
| 1371935_at | similar to transmembrane protein 9 | na |
| 1383171_at | similar to house-keeping protein | na |
| 1377338_at | similar to Rad1p | na |
| 1398581_at | similar to grey lethal osteopetrosis; grey-lethal; grey lethal | na |
| | osteroperosis | |
| 1383554_at | similar to hypothetical protein FLJ23516; gene related to | na |
| | anergy in lymphocytes | |
| 1375638_at | similar to serum deprivation response | na |
| 1382489_at | similar to WD repeat domain 11 protein | na |
| 1375533_at | similar to Hypothetical protein MGC54805 | na |
| 1384728_at | similar to checkpoint suppressor 1 | na |
| 1390697_at | similar to hypothetical protein FLJ20514 | na |
| 1371586_at | similar to mitochondrial ribosomal protein L48 | na |
| 1373535_at | similar to mena protein | na |
| 1373712_at | similar to zinc finger protein RIZ | na |
| 1395338_at | similar to leucine rich protein mLRP130 | na |
| 1388837_at | LOC363025 | na |
| 1376734_at | nephroblastoma overexpressed gene | Nov |
| 1398271_at | piccolo (presynaptic cytomatrix protein) | Pclo |
| 1383294_at | prodynorphin | Pdyn |
| 1369333_a_at | Rim2 protein | Rims2 |
| 1379281_at | uterine sensitization-associated gene 1 protein | Sostdc1 |
| 1372368_at | stress 70 protein chaperone, microsome-associated, 60 kD | Stch |
| | human homolog | |
| 1394802_at | synaptotagmin 7 | Syt7 |
| 1387013_at | kidney-specific membrane protein | Tmem27 |
| 1367598_at | transthyretin | Ttr |
| 1368858_at | UDP-glucuronosyltransferase 8 | Ugt8 |
| 1368839_at | Wolfram syndrome 1 | Wfs1 |
| 1393153_at | zinc finger protein 161 | Zfp161 |
| 1388590_at | zinc ribbon domain containing, 1 | Znrd1 |

Genes and gene fragments identified as changing significantly in animals 0.5 hours following passive avoidance training relative to controls are provided in Table 11 below.

TABLE 11

| Fragment Name | Gene Name | Gene Symbol |
|---|---|---|
| 1367999_at | (aldehyde dehydrogenase 2, hypothetical gene supported by | (Aldh2, na) |
| | NM_032416) | |
| 1372299_at | (cyclin-dependent kinase inhibitor 1C, p57, similar to p57kip2) | (Cdkn1c, na) |
| 1389868_at | (DEAD (Asp-Glu-Ala-Asp) box polypeptide 6, trehalase | (Ddx6, Treh) |
| | (brush-border membrane glycoprotein)) | |
| 1370704_at | (hypothetical gene supported by NM_013192, potassium | (Kcnj6, na) |
| | inwardly-rectifying channel, subfamily J, member 6) | |
| 1389734_x_at | (MHC class I RT1.O type 149 processed pseudogene, RT1 | (LOC360231, RT1-Aw2, RT1-T24-1) |
| | class Ib, locus Aw2, histocompatibility 2, T region locus 24) | |
| 1368263_a_at | (hypothetical gene supported by NM_012720, myelin- | (Mobp, na) |
| | associated oligodendrocytic basic protein) | |
| 1388114_at | (myosin regulatory light chain, similar to Myosin regulatory | (Mrlcb, na) |
| | light chain 2-A, smooth muscle isoform (Myosin RLC-A)) | |
| 1370428_x_at | (RT1 class I, A3, RT1 class Ib, locus Aw2, RT1 class Ib, locus | (RT1-A3, RT1-Aw2, RT1-Cl) |
| | Cl) | |
| 1387839_at | (RT1 class Ib gene, H2-TL-like, grc region (N1), RT1 class Ib | (RT1-N1, RT1-N2) |
| | gene, H2-TL-like, grc region(N2)) | |
| 1383053_x_at | (similar to zinc finger protein 91 isoform 1, zinc finger protein | (Zfp91, na) |
| | 91) | |
| 1367702_at | acetyl-coenzyme A dehydrogenase, medium chain | Acadm |
| 1369998_at | ADP-ribosylation factor 6 | Arf6 |
| 1386991_a_at | bcl-2 associated death agonist | Bad |
| 1388187_at | calcium/calmodulin-dependent protein kinase II alpha subunit | Camk2a |
| 1370185_at | contactin associated protein 1 | Cntnap1 |
| 1368536_at | ectonucleotide pyrophosphatase/phosphodiesterase 2 | Enpp2 |
| 1374320_at | coagulation factor 5 | F5 |
| 1387889_at | folate receptor 1 (adult) | Folr1 |
| 1367844_at | GTP-binding protein (G-alpha-i2) | Gnai2 |
| 1367571_a_at | insulin-like growth factor 2 | Igf2 |
| 1369322_at | potassium voltage-gated channel, Isk-related family, member 2 | Kcne2 |
| 1393260_at | WDNM1 homolog | LOC360228 |
| 1367682_at | midkine | Mdk |
| 1368302_at | homeo box, msh-like 1 | Msx1 |
| 1372457_at | mitochondrial tumor suppressor 1 | Mtus1 |
| 1377434_at | similar to membrane-type frizzled-related protein | na |
| 1396173_at | similar to D7H11orf14 protein | na |
| 1380172_at | similar to mKIAA0531 protein | na |
| 1392598_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1382024_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1383726_at | hypothetical LOC294715 | na |
| 1373790_at | similar to Aph1a-pending protein | na |
| 1377053_at | similar to RIKEN cDNA 4930444A02 | na |
| 1378997_at | similar to putative | na |
| 1381992_at | similar to RIKEN cDNA 2310042D10 | na |
| 1383615_a_at | similar to E3 ligase for inhibin receptor | na |
| 1373452_at | similar to RNA 3-terminal phosphate cyclase-like protein | na |
| 1375538_at | similar to Vinculin (Metavinculin) | na |
| 1392330_at | similar to N6-DNA-methyltransferase isoform 1 | na |
| 1383829_at | similar to HMG-box transcription factor BBX | na |
| 1393416_at | similar to RAS protein activator like 1 (GAP1 like) | na |
| 1372775_at | similar to Brd3 protein | na |
| 1385349_at | similar to centrin 4 | na |
| 1391998_at | similar to CG10042-PA | na |
| 1396278_at | similar to RIKEN cDNA 4933439F10 | na |
| 1374503_at | similar to pre B-cell leukemia transcription factor 3 | na |
| 1373238_at | similar to SPT3-associated factor 42 | na |
| 1386854_at | similar to PD2 protein | na |
| 1390251_at | similar to KIAA1822 protein | na |
| 1375211_at | similar to Ribonuclease 6 precursor | na |
| 1371822_at | similar to DNA-directed RNA polymerase III 47 kDa | na |
| | polypeptide (RNA polymerase C subunit 4) (RPC4) (RPC53) | |
| | (BN51 protein) | |
| 1378606_at | similar to hypothetical protein MGC37079 | na |
| 1391224_at | similar to 4921517L17Rik protein | na |
| 1399129_at | similar to hypothetical protein | na |
| 1376523_at | similar to retinoblastoma-binding protein 1 isoform I; | na |
| | retinoblastoma-binding protein 1 | |
| 1384280_at | similar to nucleolar protein ANKT | na |
| 1372400_at | similar to KIAA0695 protein | na |
| 1394010_at | similar to HBV pX associated protein 8 large isoform | na |
| 1378138_at | similar to chromosome 20 open reading frame 23; sorting nexin | na |
| | 23 | |
| 1390796_at | similar to glycoprotein IIb - rat | na |
| 1376734_at | nephroblastoma overexpressed gene | Nov |
| 1368939_a_at | neurotrophic tyrosine kinase, receptor, type 3 | Ntrk3 |
| 1368958_at | protein kinase C and casein kinase substrate in neurons 1 | Pacsin1 |
| 1370789_a_at | prolactin receptor | Prlr |
| 1369564_at | rad and gem related GTP binding protein 2 | Rem2 |
| 1367957_at | regulator of G-protein signalling 3 | Rgs3 |
| 1388071_x_at | RT1 class Ib, locus Aw2 | RT1-Aw2 |
| 1389089_at | RT1 class I, locus Ke4 | RT1-Ke4 |
| 1396464_at | SH3 domain protein 2A | Sh3gl2 |
| 1368606_at | solute carrier organic anion transporter family, member 1A2 | Slco1a2 |
| 1368322_at | superoxide dismutase 3, extracellular | Sod3 |
| 1379281_at | uterine sensitization-associated gene 1 protein | Sostdc1 |
| 1370518_a_at | syntaxin binding protein 1 | Stxbp1 |
| 1369627_at | synaptic vesicle glycoprotein 2 b | Sv2b |
| 1370228_at | Transferrin | Tf |
| 1367823_at | tissue inhibitor of metalloproteinase 2 | Timp2 |
| 1387013_at | kidney-specific membrane protein | Tmem27 |
| 1367598_at | transthyretin | Ttr |
| 1368839_at | Wolfram syndrome 1 | Wfs1 |
| 1387836_at | prenylated SNARE protein | Ykt6 |

Genes and gene fragments identified as changing significantly in animals 1 hour following passive avoidance training relative to controls are provided in Table 12 below.

TABLE 12

| Fragment Name | Gene Name | Gene Symbol |
|---|---|---|
| 1372299_at | (cyclin-dependent kinase inhibitor 1C, p57, similar to p57kip2) | (Cdkn1c, na) |
| 1389868_at | (DEAD (Asp-Glu-Ala-Asp) box polypeptide 6, trehalase | (Ddx6, Treh) |
| | (brush-border membrane glycoprotein)) | |
| 1370976_at | (Ras-GTPase-activating protein SH3-domain binding protein, | (G3bp, na) |
| | similar to ras-GTPase-activating protein SH3-domain binding | |
| | protein) | |
| 1371245_a_at | (hemoglobin beta chain complex, similar to Hemoglobin beta | (Hbb, na) |
| | chain, minor-form) | |
| 1386878_at | (insulin-like growth factor binding protein 2, similar to Low- | (Igfbp2, na) |
| | density lipoprotein receptor-related protein 10) | |
| 1387441_at | (neuregulin 1, potassium channel, subfamily K, member 3) | (Kcnk3, Nrg1) |
| 1389734_x_at | (MHC class I RT1.O type 149 processed pseudogene, RT1 | (LOC360231, RT1-Aw2, |
| | class Ib, locus Aw2, histocompatibility 2, T region locus 24) | RT1-T24-1) |
| 1367658_at | (SH3/ankyrin domain gene 3, hypothetical gene supported by | (Shank3, na) |
| | NM_021676) | |
| 1367853_at | (hypothetical gene supported by NM_031798, solute carrier | (Slc12a2, na) |
| | family 12, member 2) | |
| 1368587_at | apolipoprotein C-I | Apoc1 |
| 1387651_at | aquaporin 1 | Aqp1 |
| 1369077_at | N-acylsphingosine amidohydrolase (acid ceramidase) | Asah |
| 1368701_at | ATPase, Na+/K+ transporting, alpha 3 polypeptide | Atp1a3 |
| 1388237_s_at | ATPase, Ca++ transporting, plasma membrane 3 | Atp2b3 |
| 1383819_at | COP9 (constitutive photomorphogenic) homolog, subunit 2 | Cops2 |
| | (Arabidopsis thaliana) | |
| 1389974_at | casein kinase II, alpha 1 polypeptide | Csnk2a1 |
| 1370507_at | disks large-associated protein 4 | Dap4 |
| 1391406_at | degenerative spermatocyte homolog (Drosophila) | Degs |
| 1370053_at | discs, large (Drosophila) homolog-associated protein 1 | Dlgap1 |
| 1368536_at | ectonucleotide pyrophosphatase/phosphodiesterase 2 | Enpp2 |
| 1369453_at | Epsin 1 | Epn1 |
| 1374320_at | coagulation factor 5 | F5 |
| 1373829_at | fibroblast growth factor receptor 2 | Fgfr2 |
| 1370132_at | FK506 binding protein 1b | Fkbp1b |
| 1367700_at | fibromodulin | Fmod |
| 1387889_at | folate receptor 1 (adult) | Folr1 |
| 1383047_at | growth arrest specific 6 | Gas6 |
| 1370912_at | heat shock 70 kD protein 1A | Hspa1a |
| 1369124_at | 5-hydroxytryptamine (serotonin) receptor 2A | Htr2a |
| 1369860_a_at | 5-hydroxytryptamine (serotonin) receptor 2C | Htr2c |
| 1367571_a_at | insulin-like growth factor 2 | Igf2 |
| 1367648_at | insulin-like growth factor binding protein 2 | Igfbp2 |
| 1370545_at | potassium voltage-gated channel, shaker-related subfamily, | Kcna1 |
| | member 1 | |
| 1387946_at | lectin, galactoside-binding, soluble, 3 binding protein | Lgals3bp |
| 1390372_at | mitogen activated protein kinase kinase kinase 12 | Map3k12 |
| 1374933_at | melanoma cell adhesion molecule | Mcam |
| 1367682_at | midkine | Mdk |
| 1367568_a_at | matrix Gla protein | Mgp |
| 1368302_at | homeo box, msh-like 1 | Msx1 |
| 1372457_at | mitochondrial tumor suppressor 1 | Mtus1 |
| 1377434_at | similar to membrane-type frizzled-related protein | na |
| 1382024_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1371504_at | similar to 60S ribosomal protein L37a | na |
| 1376931_at | similar to Hepatocellular carcinoma-associated antigen 58 | na |
| | homolog | |
| 1383158_at | similar to HIV-1 Rev binding protein | na |
| 1391595_at | similar to acheron; death-associated LA motif protein | na |
| 1374094_at | similar to 1500031K13Rik protein | na |
| 1373790_at | similar to Aph1a-pending protein | na |
| 1383239_at | similar to RIKEN cDNA 4930572I07 gene | na |
| 1373257_at | similar to protein phosphatase 1, regulatory (inhibitory) subunit | na |
| | 1C; thymocyte ARPP; DNA segment, Chr 9, Brigham & | |
| | Womens Genetics 1012 expressed | |
| 1376299_at | similar to Retinoblastoma-binding protein 2 (RBBP-2) | na |
| 1376285_at | similar to Ced6-pending protein | na |
| 1376861_at | similar to RIKEN cDNA 1810018L05 | na |
| 1371649_at | similar to up-regulated gene 4 | na |
| 1388316_at | hypothetical LOC296207 | na |
| 1376518_at | similar to class I cytokine receptor | na |
| 1397386_at | similar to putative transcription factor ALF-4 | na |
| 1371549_at | similar to RIKEN cDNA 0710008A13 | na |
| 1377631_at | similar to alpha-3 type IX collagen | na |
| 1372839_at | similar to mitochondrial ribosomal protein S35; mitochondrial | na |
| | ribosomal protein S34 | |
| 1375954_at | similar to 8 KDa amlexanox-binding protein | na |
| 1375452_at | similar to Meningioma expressed antigen 6 (coiled-coil proline- | na |
| | rich) | |
| 1378355_a_at | similar to solute carrier family 24 (sodium/potassium/calcium | na |
| | exchanger), member 3 | |
| 1379123_at | similar to Type I transmembrane receptor (seizure-related | na |
| | protein) | |
| 1390141_at | similar to 2410004L15Rik protein | na |
| 1374442_at | similar to Splicing factor, arginine/serine-rich 9 (Pre-mRNA | na |
| | splicing factor SRp30C) | |
| 1371382_at | similar to FLJ00343 protein | na |
| 1376904_at | similar to hypothetical protein MGC33486 | na |
| 1380929_at | similar to hypothetical protein FLJ34512 | na |
| 1385511_at | similar to Seizure 6-like protein precursor | na |
| 1380548_at | similar to estrogen-related receptor beta like 1; huntingtin | na |
| | interacting protein-1 interacting protein; HIP1 protein interactor | |
| 1374313_at | similar to RIKEN cDNA 6430517J16 | na |
| 1376962_at | similar to retinoic acid-responsive protein; STRA6 | na |
| 1372860_at | similar to phospholysine phosphohistidine inorganic | na |
| | pyrophosphate phosphata (5M590) | |
| 1370946_at | nuclear factor I/X | Nfix |
| 1391455_at | conserved nuclear protein Nhn1 | Nhn1 |
| 1378010_at | origin recognition complex, subunit 4 | Orc4 |
| 1394871_at | solute carrier family 31 (copper transporters), member 1 | Slc31a1 |
| 1388534_at | solute carrier family 31 (copper transporters), member 1 | Slc31a1 |
| 1368082_at | solute carrier family 4, member 2 | Slc4a2 |
| 1368606_at | solute carrier organic anion transporter family, member 1A2 | Slco1a2 |
| 1368322_at | superoxide dismutase 3, extracellular | Sod3 |
| 1379281_at | uterine sensitization-associated gene 1 protein | Sostdc1 |
| 1396512_at | synaptogyrin 1 | Syngr1 |
| 1370228_at | Transferrin | Tf |
| 1387013_at | kidney-specific membrane protein | Tmem27 |
| 1367655_at | thymosin, beta 10 | Tmsb10 |
| 1367598_at | transthyretin | Ttr |
| 1373546_at | putative UA20 protein | Ua20 |
| 1373807_at | vascular endothelial growth factor | Vegf |
| 1368839_at | Wolfram syndrome 1 | Wfs1 |
| 1369705_at | X transporter protein 3 | Xtrp3 |
| 1399110_at | zinc finger protein 297 | Znf297 |

Genes and gene fragments identified as changing significantly in animals 2 hours following passive avoidance training relative to controls are provided in Table 13 below.

TABLE 13

| Fragment Name | Gene Name | Gene Symbol |
|---|---|---|
| 1367617_at | (aldolase A, similar to Fructose-bisphosphate aldolase A | (Aldoa, na) |
| | (Muscle-type aldolase)) | |
| 1387772_at | (calmodulin 1, hypothetical gene supported by NM_031969; | (Calm1, na) |
| | AF178845) | |
| 1369937_at | (calmodulin 1, hypothetical gene supported by NM_031969; | (Calm1, na) |
| | AF178845) | |
| 1369936_at | (calmodulin 1, hypothetical gene supported by NM_031969; | (Calm1, na) |
| | AF178845) | |
| 1373470_at | (beta-catenin, similar to beta-catenin; catenin beta) | (Catnb, na) |
| 1372299_at | (cyclin-dependent kinase inhibitor 1C, p57, similar to p57kip2) | (Cdkn1c, na) |
| 1386921_at | (carboxypeptidase E, similar to Carboxypeptidase H precursor | (Cpe, na) |
| | (CPH) (Carboxypeptidase E) (CPE) (Enkephalin convertase) | |
| | (Prohormone processing carboxypeptidase)) | |
| 1369939_at | (cytochrome c, somatic, similar to Cytochrome c, somatic) | (Cycs, na) |
| 1389868_at | (DEAD (Asp-Glu-Ala-Asp) box polypeptide 6, trehalase | (Ddx6, Treh) |
| | (brush-border membrane glycoprotein)) | |
| 1370109_s_at | (eukaryotic translation elongation factor 1 alpha 1, eukaryotic | (Eef1a1, Eef1a2, na) |
| | translation elongation factor 1 alpha 2, hypothetical | |
| | LOC293924, similar to Elongation factor 1-alpha 1 (EF-1- | |
| | alpha-1) (Elongation factor 1 A-1) (eEF1A-1) (Elongation | |
| | factor Tu) (EF-Tu)) | |
| 1388110_at | (eukaryotic translation elongation factor 1 alpha 1, eukaryotic | (Eef1a1, Eef1a2, na) |
| | translation elongation factor 1 alpha 2, hypothetical | |
| | LOC293924, similar to Elongation factor 1-alpha 1 (EF-1- | |
| | alpha-1) (Elongation factor 1 A-1) (eEF1A-1) (Elongation | |
| | factor Tu) (EF-Tu)) | |
| 1387570_at | (endo-alpha-mannosidase, hypothetical gene supported by | (Enman, na) |
| | NM_080785) | |
| 1367575_at | (enolase 1, alpha, similar to Alpha enolase (2-phospho-D- | (Eno1, na) |
| | glycerate hydro-lyase) (Non-neural enolase) (NNE) (Enolase | |
| | 1)) | |
| 1398829_at | (FK506 binding protein 2, FK506-binding protein 1a) | (Fkbp1a, Fkbp2) |
| 1398828_at | (FK506 binding protein 2, FK506-binding protein 1a) | (Fkbp1a, Fkbp2) |
| 1367565_a_at | (ferritin, heavy polypeptide 1, similar to ferritin, heavy | (Fth1, na) |
| | polypeptide 1; Ferritin subunit H) | |
| 1367557_s_at | (glyceraldehyde-3-phosphate dehydrogenase, similar to | (Gapd, na) |
| | glyceraldehyde-3-phosphate dehydrogenase, similar to | |
| | glyceraldehyde-3-phosphate dehydrogenase (phosphorylating) | |
| | (EC 1.2.1.12) - mouse, similar to glyceraldehyde-3-phosphate- | |
| | dehydrogenase (EC 1.2.1.12) | |
| 1368353_at | (glial fibrillary acidic protein, hypothetical gene supported by | (Gfap, na) |
| | L27219; NM_017009) | |
| 1372002_at | (gap junction membrane channel protein alpha 1, hypothetical | (Gja1, na) |
| | gene supported by NM_012567) | |
| 1375705_at | (guanine nucleotide binding protein, beta 1, similar to guanine | (Gnb1, na) |
| | nucleotide-binding protein, beta-1 subunit) | |
| 1381996_at | (glycoprotein m6b, hypothetical gene supported by | (Gpm6b, na) |
| | NM_138846) | |
| 1371245_a_at | (hemoglobin beta chain complex, similar to Hemoglobin beta | (Hbb, na) |
| | chain, minor-form) | |
| 1367553_x_at | (hemoglobin beta chain complex, similar to Hemoglobin beta | (Hbb, na) |
| | chain, minor-form) | |
| 1371102_x_at | (hemoglobin beta chain complex, similar to Hemoglobin beta | (Hbb, na) |
| | chain, minor-form) | |
| 1398240_at | (heat shock protein 8, similar to Heat shock cognate 71 kDa | (Hspa8, na) |
| | protein) | |
| 1381030_at | (similar to step II splicing factor SLU7; DNA segment, Chr 11, | (LOC303057, Slu7) |
| | ERATO Doi 730, expressed; DNA segment, Chr 3, Brigham & | |
| | Womens Genetics 0878 expressed, step II splicing factor SLU7 | |
| | (S. cerevisiae)) | |
| 1371003_at | (microtubule-associated protein 1b, similar to Microtubule- | (Map1b, na) |
| | associated protein 1B (MAP 1B) (Neuraxin)) | |
| 1373363_at | (microtubule-associated protein 1b, similar to Microtubule- | (Map1b, na) |
| | associated protein 1B (MAP 1B) (Neuraxin)) | |
| 1382522_at | (hypothetical LOC293831, matrin 3) | (Matr3, na) |
| 1367609_at | (macrophage migration inhibitory factor, similar to macrophage | (Mif, na) |
| | migration inhibitory factor) | |
| 1370434_a_at | (hypothetical gene supported by NM_012720, myelin- | (Mobp, na) |
| | associated oligodendrocytic basic protein) | |
| 1388152_at | (hypothetical gene supported by NM_013066, microtubule- | (Mtap2, na) |
| | associated protein 2) | |
| 1378264_at | (nuclear autoantigenic sperm protein, similar to Nasp protein) | (Nasp, na) |
| 1370724_a_at | (hypothetical gene supported by AB060652; NM_012988, | (Nfia, na) |
| | nuclear factor I/A) | |
| 1370517_at | (hypothetical gene supported by NM_153735, neuronal | (Nptx1, Ntsr2, na) |
| | pentraxin 1, neurotensin receptor 2) | |
| 1368875_a_at | (hypothetical gene supported by NM_053846, neurexin 2) | (Nrxn2, na) |
| 1398841_at | (ras-related protein, similar to Ras-related protein Rab-1A | (Rab1, na) |
| | (YPT1-related protein)) | |
| 1372707_at | (RAB6, member RAS oncogene family, similar to RAB6, | (Rab6, na) |
| | member RAS oncogene family) | |
| 1367625_at | (hypothetical LOC291740, ribosomal protein L10, similar to | (Rpl10, na) |
| | 60S ribosomal protein L10 (QM protein homolog)) | |
| 1367610_at | (ribosomal protein L19, similar to 60S ribosomal protein L19) | (Rpl19, na) |
| 1398830_at | (ribosomal protein L28, similar to ribosomal protein L28) | (Rpl28, na) |
| 1371300_at | (ribosomal protein L3, similar to 60S RIBOSOMAL PROTEIN | (Rpl3, na) |
| | L3 (L4)) | |
| 1367634_at | (ribosomal protein L31, similar to ribosomal protein L31) | (Rpl31, na) |
| 1398760_at | (ribosomal protein L35a, similar to ribosomal protein L35a) | (Rpl35a, na) |
| 1398789_at | (ribosomal protein L37, similar to ribosomal protein L37) | (Rpl37, na) |
| 1371301_at | (ribosomal protein L9, similar to 60S RIBOSOMAL PROTEIN | (Rpl9, na) |
| | L9, similar to ribosomal protein L9) | |
| 1371307_at | (ribosomal protein, large, P1, similar to 60S ACIDIC | (Rplp1, na) |
| | RIBOSOMAL PROTEIN P1) | |
| 1371340_at | (ribosomal protein, large P2, similar to 60S ACIDIC | (Rplp2, na) |
| | RIBOSOMAL PROTEIN P2) | |
| 1367630_at | (ribosomal protein S11, similar to 40S ribosomal protein S11) | (Rps11, na) |
| 1388296_at | (ribosomal protein S18, similar to ribosomal protein S18, | (Rps18, na) |
| | similar to ribosomal protein S18, cytosolic [validated] - rat) | |
| 1375219_a_at | (ribosomal protein S2, similar to 40S ribosomal protein S2) | (Rps2, na) |
| 1370242_at | (ribosomal protein S23, similar to ribosomal protein S23) | (Rps23, na) |
| 1367685_at | (ribosomal protein S27a, similar to ribosomal protein S27a) | (Rps27a, na) |
| 1398751_at | (hypothetical LOC298661, ribosomal protein S7) | (Rps7, na) |
| 1368098_a_at | (SNRPN upstream reading frame, similar to small nuclear | (Snrpn, Snurf, na) |
| | ribonucleoprotein-associated protein N - rat, small nuclear | |
| | ribonucleoprotein N) | |
| 1367603_at | (similar to triosephosphate isomerase 1, triosephosphate | (Tpi1, na) |
| | isomerase 1) | |
| 1367583_at | (similar to Translationally controlled tumor protein (TCTP) | (Tpt1, na) |
| | (p23) (21 kDa polypeptide) (p21) (Lens epithelial protein), | |
| | tumor protein, translationally-controlled 1) | |
| 1367579_a_at | (hypothetical gene supported by NM_022298; NM_022298; | (Tuba1, na) |
| | V01227, tubulin, alpha 1) | |
| 1387862_at | (similar to 14-3-3 PROTEIN TAU (14-3-3 PROTEIN THETA), | (Ywhaq, na) |
| | similar to 14-3-3 protein tau (14-3-3 protein theta) (14-3-3 | |
| | protein T-cell) (HS1 protein), tyrosine 3- | |
| | monooxygenase/tryptophan 5-monooxygenase activation | |
| | protein, theta polypeptide) | |
| 1383054_at | (similar to zinc finger protein 91 isoform 1, zinc finger protein | (Zfp91, na) |
| | 91) | |
| 1383053_x_at | (similar to zinc finger protein 91 isoform 1, zinc finger protein | (Zfp91, na) |
| | 91) | |
| 1383052_a_at | (similar to zinc finger protein 91 isoform 1, zinc finger protein | (Zfp91, na) |
| | 91) | |
| 1398835_at | actin, beta | Actb |
| 1398836_s_at | actin, beta | Actb |
| 1378566_at | adenylate cyclase 1 | Adcy1 |
| 1370584_a_at | adenosine A1 receptor | Adora1 |
| 1387861_at | amino-terminal enhancer of split | Aes |
| 1398753_at | aldo-keto reductase family 1, member A1 | Akr1a1 |
| 1386998_at | aldolase C, fructose-biphosphate | Aldoc |
| 1369063_at | acidic (leucine-rich) nuclear phosphoprotein 32 family, member A | Anp32a |
| 1389307_at | amyloid beta (A4) precursor-like protein 1 | Aplp1 |
| 1370862_at | apolipoprotein E | Apoe |
| 1371571_at | amyloid beta (A4) precursor protein | App |
| 1372190_at | aquaporin 4 | Aqp4 |
| 1388997_at | ADP-ribosylation factor 3 | Arf3 |
| 1368266_at | arginase 1 | Arg1 |
| 1394318_at | cyclic AMP phosphoprotein, 19 kDa | Arpp19 |
| 1386911_at | ATPase, Na+K+ transporting, alpha 2 | Atp1a2 |
| 1367814_at | ATPase, Na+/K+ transporting, beta 1 polypeptide | Atp1b1 |
| 1386937_at | ATPase, Na+/K+ transporting, beta 1 polypeptide | Atp1b1 |
| 1370275_at | ATP synthase, H+ transporting, mitochondrial F1 complex, beta | Atp5b |
| | polypeptide | |
| 1370918_a_at | ATP synthase, H+ transporting, mitochondrial F1 complex, | Atp5c1 |
| | gamma polypeptide 1 | |
| 1370284_at | ATP synthase, H+ transporting, mitochondrial F1 complex, | Atp5e |
| | epsilon subunit | |
| 1367620_at | ATP synthase, H+ transporting, mitochondrial F0 complex, | Atp5g3 |
| | subunit c (subunit 9) isoform 3 | |
| 1398755_at | ATPase, H+ transporting, lysosomal (vacuolar proton pump) 16 kDa | Atp61 |
| 1387127_at | attractin | Atrn |
| 1386991_a_at | bcl-2 associated death agonist | Bad |
| 1398350_at | brain abundant, membrane attached signal protein 1 | Basp1 |
| 1368509_at | Bardet-Biedl syndrome 2 (human) | Bbs2 |
| 1387133_at | calbindin 2 | Calb2 |
| 1370246_at | calmodulin 2 | Calm2 |
| 1370873_at | calmodulin 3 | Calm3 |
| 1387032_at | cholecystokinin | Cck |
| 1387038_at | copper chaperone for superoxide dismutase | Ccs |
| 1398827_at | CD 81 antigen | Cd81 |
| 1370184_at | cofilin 1 | Cfl1 |
| 1387235_at | chromogranin A | Chga |
| 1368287_at | chimerin (chimaerin) 1 | Chn1 |
| 1376800_at | chimerin (chimaerin) 2 | Chn2 |
| 1367740_at | creatine kinase, brain | Ckb |
| 1390566_a_at | creatine kinase, mitochondrial 1, ubiquitous | Ckmt1 |
| 1367607_at | cytochrome c oxidase subunit IV isoform 1 | Cox4i1 |
| 1370861_at | cytochrome c oxidase, subunit VIa, polypeptide 1 | Cox6a1 |
| 1367757_at | cytochrome oxidase subunit VIc | Cox6c |
| 1371387_at | cytochrome c oxidase subunit VIIb | Cox7b |
| 1388113_at | cytochrome c oxidase, subunit VIIIa | Cox8a |
| 1369912_at | v-crk sarcoma virus CT10 oncogene homolog (avian) | Crk |
| 1389974_at | casein kinase II, alpha 1 polypeptide | Csnk2a1 |
| 1370855_at | cystatin C | Cst3 |
| 1371465_at | cortactin isoform B | Cttn |
| 1388177_at | dendrin | Ddn |
| 1368292_at | dynamin 1 | Dnm1 |
| 1371694_at | dihydropyrimidinase-like 2 | Dpysl2 |
| 1368867_at | GERp95 | Eif2c2 |
| 1368866_at | GERp95 | Eif2c2 |
| 1372735_at | eukaryotic translation initiation factor 3 subunit k | eIF3k |
| 1382168_at | ELAV (embryonic lethal, abnormal vision, Drosophila)-like 3 | Elavl3 |
| | (Hu antigen C) | |
| 1388666_at | ectodermal-neural cortex 1 | Enc1 |
| 1382710_at | ectodermal-neural cortex 1 | Enc1 |
| 1370341_at | enolase 2, gamma | Eno2 |
| 1368536_at | ectonucleotide pyrophosphatase/phosphodiesterase 2 | Enpp2 |
| 1374320_at | coagulation factor 5 | F5 |
| 1371316_at | Finkel-Biskis-Reilly murine sarcoma virusubiquitously | Fau |
| | expressed | |
| 1370132_at | FK506 binding protein 1b | Fkbp1b |
| 1397587_at | glucose 6 phosphatase, catalytic, 3 | G6pc3 |
| 1371057_at | gamma-aminobutyric acid A receptor, alpha 5 | Gabra5 |
| 1369903_at | gamma-aminobutyric acid receptor, subunit beta 3 | Gabrb3 |
| 1367930_at | growth associated protein 43 | Gap43 |
| 1383047_at | growth arrest specific 6 | Gas6 |
| 1375613_at | GATA binding protein 6 | Gata6 |
| 1367632_at | glutamine synthetase 1 | Glul |
| 1370091_at | heterotrimeric guanine nucleotide-binding protein alpha q | Gnaq |
| | subunit | |
| 1369897_s_at | GNAS complex locus | Gnas |
| 1369410_at | golgi SNAP receptor complex member 1 | Gosr1 |
| 1375438_at | golgi SNAP receptor complex member 2 | Gosr2 |
| 1371363_at | glycerol-3-phosphate dehydrogenase 1 (soluble) | Gpd1 |
| 1371392_at | glucose phosphate isomerase | Gpi |
| 1373773_at | glycoprotein m6a | Gpm6a |
| 1389132_at | huntingtin interacting protein 1 | Hip1 |
| 1387387_at | hippocalcin | Hpca |
| 1372701_at | heat shock protein 1, alpha | Hspca |
| 1375336_at | heat shock 90 kDa protein 1, beta | Hspcb |
| 1367571_a_at | insulin-like growth factor 2 | Igf2 |
| 1370975_at | jumonji domain containing 1A | Jmjd1a |
| 1369741_at | potassium inwardly-rectifying channel, subfamily J, member 3 | Kcnj3 |
| 1369848_at | potassium channel, alpha subunit (kv9.2 gene) | Kcns2 |
| 1382787_at | kinesin family member 5A | Kif5a |
| 1370770_s_at | kit ligand | Kitl |
| 1388856_at | kit ligand | Kitl |
| 1370886_a_at | kinesin light chain 1 | Klc1 |
| 1368504_at | lysosomal membrane glycoprotein 1 | Lamp1 |
| 1388024_s_at | lamina-associated polypeptide 1C | Lap1c |
| 1370218_at | lactate dehydrogenase B | Ldhb |
| 1374307_at | CaM-kinase II inhibitor alpha | LOC287005 |
| 1393606_at | hypothetical protein LK44 | LOC362219 |
| 1389127_at | MAD homolog 3 (Drosophila) | Madh3 |
| 1387834_at | megakaryocyte-associated tyrosine kinase | Matk |
| 1368810_a_at | myelin basic protein | Mbp |
| 1387341_a_at | myelin basic protein | Mbp |
| 1367682_at | midkine | Mdk |
| 1381174_at | similar to Hypothetical protein KIAA0141 | MGC72566 |
| 1371564_at | Unknown (protein for MGC: 72933) | MGC72933 |
| 1398324_at | similar to 60S ribosomal protein L18a | MGC72957 |
| 1376315_at | similar to putative alpha-mannosidase | MGC93956 |
| 1370129_at | meningioma expressed antigen 5 (hyaluronidase) | Mgea5 |
| 1373002_at | similar to 28S ribosomal protein S9, mitochondrial precursor | Mrps9 |
| | (MRP-S9) | |
| 1370124_at | metallothionein 3 | Mt3 |
| 1371841_at | myotrophin | Mtpn |
| 1375632_at | similar to tweety homolog 2 | na |
| 1391416_at | similar to gene trap ankyrin repeat; serologically defined breast | na |
| | cancer antigen NY-BR-16 | |
| 1371320_at | similar to E25B protein | na |
| 1371334_at | similar to integral membrane protein 2C | na |
| 1392974_at | similar to WASP family 1 | na |
| 1371393_at | similar to calsyntenin-1 protein | na |
| 1388299_at | similar to Cytosolic acyl coenzyme A thioester hydrolase, | na |
| | inducible (Long chain acyl-CoA thioester hydrolase) (Long | |
| | chain acyl-CoA hydrolase) (CTE-I) (LACH2) (ACH2) | |
| 1392598_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1371347_at | similar to RIKEN cDNA B230114J08 | na |
| 1367536_at | similar to MMRP19 | na |
| 1371344_at | (similar to 60S RIBOSOMAL PROTEIN L27A, similar to 60S | na |
| | ribosomal protein L27a) | |
| 1388632_at | similar to Ras-related protein Rab-6B | na |
| 1371321_at | similar to Uroplakin Ia (UPIa) (UPKa) | na |
| 1398893_at | similar to Nedd4 WW domain-binding protein 5 | na |
| 1371474_at | similar to Mtch1 protein | na |
| 1377615_at | similar to hypothetical protein | na |
| 1372249_at | similar to Ac1254 | na |
| 1376253_at | similar to lymphocyte antigen 6H | na |
| 1382064_at | similar to 4933407C03Rik protein | na |
| 1393881_at | similar to N-terminal aceyltransferase 1 | na |
| 1391170_at | (similar to Gene trap ROSA 26 antisense, Philippe Soriano, | na |
| | similar to mKIAA1757 protein) | |
| 1377993_at | similar to C321D2.5 (novel protein) | na |
| 1392972_at | similar to Triple functional domain protein (PTPRF interacting | na |
| | protein) | |
| 1372338_at | similar to RIKEN cDNA 1500034J20 | na |
| 1382875_at | similar to KIAA1838 protein | na |
| 1371507_at | similar to 4921517L17Rik protein | na |
| 1371504_at | similar to 60S ribosomal protein L37a | na |
| 1389904_at | similar to actin-related protein 2 | na |
| 1371573_at | similar to large subunit ribosomal protein L36a | na |
| 1371761_at | similar to ribosomal protein L34; 60S ribosomal protein L34 | na |
| 1370903_a_at | similar to Ndr3 protein | na |
| 1371319_at | similar to E25B protein | na |
| 1393409_at | similar to amisyn | na |
| 1374856_at | similar to RIKEN cDNA 4930556P03 | na |
| 1388303_at | similar to 60S ribosomal protein L26 | na |
| 1379401_a_at | similar to 5730501N20Rik protein | na |
| 1371312_at | (similar to ethanol induced 6, similar to septin 10 isoform 1) | na |
| 1373266_at | similar to downregulated in renal cell carcinoma | na |
| 1371767_at | similar to SMARCD1 protein | na |
| 1389199_at | similar to Ab2-095 | na |
| 1383726_at | hypothetical LOC294715 | na |
| 1396996_at | similar to Ubiquitin carboxyl-terminal hydrolase 1 (Ubiquitin | na |
| | thiolesterase 1) (Ubiquitin-specific processing protease 1) | |
| | (Deubiquitinating enzyme 1) (hUBP) | |
| 1389600_at | similar to Coatomer gamma-2 subunit (Gamma-2 coat protein) | na |
| | (Gamma-2 COP) | |
| 1390940_at | similar to mKIAA0569 protein | na |
| 1381967_at | similar to RNA-binding region (RNP1, RRM) containing 2; | na |
| | splicing factor (CC1.3); coactivator of activating protein-1 and | |
| | estrogen receptors | |
| 1376861_at | similar to RIKEN cDNA 1810018L05 | na |
| 1378978_a_at | similar to KIAA1205 protein | na |
| 1376120_at | similar to pancreatitis-induced protein 49 | na |
| 1392051_at | similar to hypothetical protein FLJ14681 | na |
| 1396803_at | similar to Tho2 | na |
| 1371297_at | (similar to 60S ribosomal protein L7a (Surfeit locus protein 3) | na |
| | (PLA-X polypeptide), similar to Rpl7a protein) | |
| 1376780_at | similar to RIKEN cDNA 2610204K14 | na |
| 1391855_at | similar to B-cell CLL/lymphoma 7A | na |
| 1391434_at | similar to FKSG24 | na |
| 1390103_at | similar to PHD finger protein 2 | na |
| 1373756_at | similar to BM88 antigen | na |
| 1391309_at | similar to Gliacolin | na |
| 1384323_at | similar to proteasome 26S ATPase subunit 6 | na |
| 1379645_at | similar to polybromo-1D | na |
| 1384255_at | similar to Gamma-soluble NSF attachment protein (SNAP- | na |
| | gamma) (N-ethylmaleimide-sensitive factor attachment protein, | |
| | gamma) | |
| 1379292_at | similar to 5730420B22Rik protein | na |
| 1394093_at | similar to ATP-dependent chromatin remodeling protein | na |
| | SNF2H | |
| 1376811_a_at | similar to cleavage and polyadenylation specific factor 6, 68 kD | na |
| | subunit; pre-mRNA cleavage factor Im (68 kD); cleavage and | |
| | polyadenylation specific factor 6, 68 kD subunit; pre-mRNA | |
| | cleavage factor I, 68 kD subunit | |
| 1375675_at | similar to muscleblind-like protein 2 isoform 1; muscleblind- | na |
| | like protein MBLL39 | |
| 1371642_at | similar to translation initiation factor eIF-4A II-mouse | na |
| 1384009_at | similar to nuclear prelamin A recognition factor isoform a | na |
| 1389564_at | similar to cyclin L1; cyclin L ania-6a | na |
| 1377926_at | similar to Centaurin gamma 2 | na |
| 1383023_at | similar to Ubiquitin-conjugating enzyme E2 H (Ubiquitin- | na |
| | protein ligase H) (Ubiquitin carrier protein H) (UBCH2) (E2- | |
| | 20K) | |
| 1393383_at | similar to Rho-GDI3 gene product | na |
| 1378433_at | similar to RIKEN cDNA 0610012A05 | na |
| 1379101_at | similar to DEAD/H box polypeptide 36 protein | na |
| 1376137_at | similar to evectin-2 | na |
| 1374596_at | similar to RIKEN cDNA 1810043G02; DNA segment, Chr 10, | na |
| | Johns Hopkins University 13, expressed | |
| 1378259_at | similar to Tardbp protein | na |
| 1376702_at | similar to KIAA0027 protein | na |
| 1372071_at | similar to 8D6 antigen | na |
| 1377940_at | hypothetical LOC287534 | na |
| 1371988_at | similar to Man9-mannosidase | na |
| 1374500_at | similar to RIKEN cDNA E230011A21 gene | na |
| 1374864_at | similar to sprouty-2 | na |
| 1392246_at | similar to RA175 | na |
| 1373814_at | similar to mKIAA1002 protein | na |
| 1374547_at | similar to RIKEN cDNA A930015D22 | na |
| 1380435_at | similar to Shoc2 protein | na |
| 1389693_at | similar to hypothetical protein 3010020C06 | na |
| 1371738_at | similar to hypothetical protein FLJ10375 | na |
| 1379729_at | similar to cleavage and polyadenylation specific factor 6, 68 kD | na |
| | subunit; pre-mRNA cleavage factor Im (68 kD); cleavage and | |
| | polyadenylation specific factor 6, 68 kD subunit; pre-mRNA | |
| | cleavage factor I, 68 kD subunit | |
| 1376916_at | similar to zinc finger and BTB domain containing 2 | na |
| 1378979_x_at | similar to KIAA1205 protein | na |
| 1388535_at | similar to erythroid differentiation-related factor 1 | na |
| 1389556_at | similar to KAP3A | na |
| 1385073_at | similar to hypothetical protein DJ667H12.2 | na |
| 1389646_at | similar to rabkinesin-6 | na |
| 1383354_a_at | similar to 5730501N20Rik protein | na |
| 1395479_at | similar to BRIX | na |
| 1393866_at | similar to Tnfrsf25 protein | na |
| 1394438_at | similar to ARP1 actin-related protein 1 homolog A; actin- | na |
| | related protein 1 alpha; actin-related protein 1 homolog A | |
| | (yeast); Arp1 contractin alpha; Arp1 centractin alpha | |
| 1380235_at | similar to heparan sulfate 2-sulfotransferase | na |
| 1373538_at | similar to Ubiquitin carboxyl-terminal hydrolase 1 (Ubiquitin | na |
| | thiolesterase 1) (Ubiquitin-specific processing protease 1) | |
| | (Deubiquitinating enzyme 1) (hUBP) | |
| 1399118_at | similar to RIKEN cDNA 4121402D02 | na |
| 1388315_at | similar to neuronal protein 15.6 | na |
| 1367956_at | neurochondrin | Ncdn |
| 1370229_at | N-myc downstream regulated 4 | Ndr4 |
| 1387121_a_at | N-myc downstream-regulated gene 2 | Ndrg2 |
| 1387204_at | neuronal growth regulator 1 | Negr1 |
| 1388167_at | nuclear factor I/B | Nfib |
| 1370059_at | neurofilament, light polypeptide | Nfl |
| 1369999_a_at | neuronatin | Nnat |
| 1387099_at | natriuretic peptide receptor 2 | Npr2 |
| 1376362_at | neuronal pentraxin receptor | Nptxr |
| 1370211_at | neurogranin | Nrgn |
| 1391625_at | neural Wiskott-Aldrich syndrome protein | N-WASP |
| 1369008_a_at | olfactomedin 1 | Olfm1 |
| 1387927_a_at | olfactomedin 1 | Olfm1 |
| 1392480_at | poly(A) binding protein, nuclear 1 | Pabpn1 |
| 1386891_at | phosphatidylethanolamine binding protein | Pbp |
| 1388551_at | protocadherin gamma subfamily C, 3 | Pcdhgc3 |
| 1368145_at | Purkinje cell protein 4 | Pcp4 |
| 1367835_at | proprotein convertase subtilisin/kexin type 1 inhibitor | Pcsk1n |
| 1369070_at | peroxisomal biogenesis factor 12 | Pex12 |
| 1386864_at | phosphoglycerate mutase 1 | Pgam1 |
| 1388318_at | phosphoglycerate kinase 1 | Pgk1 |
| 1369976_at | dynein, cytoplasmic, light chain 1 | Pin |
| 1388251_at | protein kinase C, lambda | Pkcl |
| 1369105_a_at | protein kinase (cAMP dependent, catalytic) inhibitor beta | Pkib |
| 1387112_at | proteolipid protein | Plp |
| 1398850_at | peptidylprolyl isomerase A | Ppia |
| 1368277_at | protein phosphatase 3, catalytic subunit, alpha isoform | Ppp3ca |
| 1373479_at | protein phosphatase 3, catalytic subunit, alpha isoform | Ppp3ca |
| 1372982_at | protein phospatase 3, regulatory subunit B, alpha isoform, type 1 | Ppp3r1 |
| 1370585_a_at | protein kinase C, beta 1 | Prkcb1 |
| 1369089_at | protein kinase C, gamma | Prkcc |
| 1370156_at | prion protein | Prnp |
| 1367851_at | prostaglandin D2 synthase | Ptgds |
| 1383117_at | peroxisomal membrane protein 4 | Pxmp4 |
| 1392681_at | RAB2, member RAS oncogene family | Rab2 |
| 1370539_at | RAB8B, member RAS oncogene family | Rab8b |
| 1369780_at | RAS protein-specific guanine nucleotide-releasing factor 2 | Rasgrf2 |
| 1370130_at | plysia ras-related homolog A2 | Rhoa |
| 1368211_at | ribosomal protein S14 | Rps14 |
| 1386874_at | ribosomal protein S15 | Rps15 |
| 1398775_at | ribosomal protein S15a | Rps15a |
| 1367639_a_at | ribosomal protein S2 | Rps2 |
| 1398852_at | ribosomal protein S21 | Rps21 |
| 1387890_at | ribosomal protein S29 | Rps29 |
| 1371299_at | ribosomal protein S3 | Rps3 |
| 1367606_at | ribosomal protein S3a | Rps3a |
| 1367573_at | ribosomal protein S6 | Rps6 |
| 1368097_a_at | reticulon 1 | Rtn1 |
| 1368888_a_at | reticulon 4 | Rtn4 |
| 1386903_at | S100 protein, beta polypeptide | S100b |
| 1387926_at | sterol-C5-desaturase (fungal ERG3, delta-5-desaturase)-like | Sc5d |
| 1367668_a_at | stearoyl-Coenzyme A desaturase 2 | Scd2 |
| 1388253_at | stearoyl-Coenzyme A desaturase 2 | Scd2 |
| 1383435_at | sodium channel, voltage-gated, type III, beta | Scn3b |
| 1387016_a_at | stromal cell derived factor receptor 1 | Sdfr1 |
| 1367593_at | selenoprotein W, muscle 1 | Sepw1 |
| 1367802_at | serum/glucocorticoid regulated kinase | Sgk |
| 1370803_at | SNAP25 interacting protein 30 | Sip30 |
| 1368987_at | solute carrier family 17 (sodium-dependent inorganic phosphate | Slc17a7 |
| | cotransporter), member 7 | |
| 1368565_at | solute carrier family 1 (glial high affinity glutamate | Slc1a3 |
| | transporter), member 3 | |
| 1371889_at | solute carrier family 22 (organic cation transporter), member 17 | Slc22a17 |
| 1388112_at | solute carrier family 25 (mitochondrial adenine nucleotide | Slc25a4 |
| | translocator) member 4 | |
| 1370848_at | solute carrier family 2 (facilitated glucose transporter), member 1 | Slc2a1 |
| 1369680_at | solute carrier family 2 (facilitated glucose transporter), member | Slc2a13 |
| | 13 | |
| 1370159_at | SWI/SNF related, matrix associated, actin dependent regulator | Smarcd2 |
| | of chromatin, subfamily d, member 2 | |
| 1387073_at | synaptosomal-associated protein | Snap25 |
| 1373865_at | synaptosomal-associated protein, 91 kDa homolog (mouse) | Snap91 |
| 1371568_at | synuclein, beta | Sncb |
| 1398245_at | synuclein, gamma | Sncg |
| 1379281_at | uterine sensitization-associated gene 1 protein | Sostdc1 |
| 1386865_at | SPARC-like 1 (mast9, hevin) | Sparcl1 |
| 1382649_at | Sjogren syndrome antigen B | Ssb |
| 1369718_at | TRAP-complex gamma subunit | Ssr3 |
| 1370224_at | signal transducer and activator of transcription 3 | Stat3 |
| 1372368_at | stress 70 protein chaperone, microsome-associated, 60 kD | Stch |
| | human homolog | |
| 1388811_at | synapsin II | Syn2 |
| 1396512_at | synaptogyrin 1 | Syngr1 |
| 1368276_at | synaptophysin | Syp |
| 1373896_at | synaptotagmin 1 | Syt1 |
| 1388307_at | tumor differentially expressed 1, like | Tde2 |
| 1370228_at | Transferrin | Tf |
| 1374257_at | T-cell lymphoma invasion and metastasis 1 | Tiam1 |
| 1371702_at | transmembrane 4 superfamily member 2 | Tm4sf2 |
| 1393418_at | tropomodulin 2 | Tmod2 |
| 1387883_a_at | thymosin beta-4 | Tmsb4x |
| 1370288_a_at | tropomyosin 1, alpha | Tpm1 |
| 1387617_at | tropomyosin 3, gamma | Tpm3 |
| 1367976_at | tripeptidyl peptidase II | Tpp2 |
| 1384163_at | transformation related protein 53 inducible nuclear protein 1 | Trp53inp1 |
| 1367598_at | transthyretin | Ttr |
| 1371618_s_at | tubulin, beta 3 | Tubb3 |
| 1398754_at | ubiquitin A-52 residue ribosomal protein fusion product 1 | Uba52 |
| 1370274_at | polyubiquitin | Ubb |
| 1386852_x_at | polyubiquitin | Ubb |
| 1370238_at | upregulated during skeletal muscle growth 5 | Usmg5 |
| 1368839_at | Wolfram syndrome 1 | Wfs1 |
| 1398800_at | tyrosine 3-monooxygenase/tryptophan 5-monooxygenase | Ywhab |
| | activation protein, beta polypeptide | |
| 1398851_at | tyrosine 3-monooxygenase/tryptophan 5-monooxygenase | Ywhae |
| | activation protein, epsilon polypeptide | |
| 1386866_at | tyrosine 3-monooxgenase/tryptophan 5-monooxgenase | Ywhag |
| | activation protein, gamma polypeptide | |
| 1367693_at | tyrosine 3-monooxygenase/tryptophan 5-monooxygenase | Ywhah |
| | activation protein, eta polypeptide | |

Genes and gene fragments identified as changing significantly in animals 3 hours following passive avoidance training relative to controls are provided in Table 14 below.

TABLE 14

| Fragment Name | Gene Name | Gene Symbol |
|---|---|---|
| 1369827_at | (calsyntenin 3, hypothetical gene supported by NM_134376) | (Cstn3, na) |
| 1395237_at | (eukaryotic translation initiation factor 5B, similar to | (Eif5b, na) |
| | Translation initiation factor IF-2) | |
| 1387302_at | adenylate cyclase activating polypeptide 1 receptor 1 | Adcyap1r1 |
| 1368868_at | A kinase (PRKA) anchor protein (gravin) 12 | Akap12 |
| 1387673_a_at | annexin A6 | Anxa6 |
| 1368677_at | brain derived neurotrophic factor | Bdnf |
| 1387170_at | casein kinase II, alpha 1 polypeptide | Csnk2a1 |
| 1370956_at | decorin | Dcn |
| 1370533_at | discs, large (Drosophila) homolog-associated protein 3 | Dlgap3 |
| 1368536_at | ectonucleotide pyrophosphatase/phosphodiesterase 2 | Enpp2 |
| 1369371_a_at | gamma-aminobutyric acid (GABA) B receptor, 1 | Gabbr1 |
| 1369167_at | glial cell line derived neurotrophic factor family receptor alpha 2 | Gfra2 |
| 1390384_at | dolichyl-phosphate (UDP-N-acetylglucosamine) N- | H2afx |
| | acetylglucosaminephosphotransferase 1 (GlcNAc-1-P | |
| | transferase) | |
| 1367571_a_at | insulin-like growth factor 2 | Igf2 |
| 1387366_at | interleukin enhancer binding factor 3 | Ilf3 |
| 1368819_at | integrin beta 1 | Itgb1 |
| 1387689_at | potassium voltage-gated channel, shaker-related subfamily, beta | Kcnab2 |
| | member 2 | |
| 1387657_at | kinesin family member 3C | Kif3c |
| 1370853_at | CaM-kinase II inhibitor alpha | LOC287005 |
| 1376253_at | similar to lymphocyte antigen 6H | na |
| 1382024_at | (similar to DnaJ (Hsp40) homolog, subfamily B, member 6, | na |
| | similar to DnaJ homolog subfamily B member 6 (Heat shock | |
| | protein J2) (HSJ-2) (MRJ) (mDj4), similar to mDj4) | |
| 1397632_at | similar to neuronal transmembrane protein Slitrk1 | na |
| 1395835_at | similar to CGI-130 protein | na |
| 1376780_at | similar to RIKEN cDNA 2610204K14 | na |
| 1390103_at | similar to PHD finger protein 2 | na |
| 1383966_at | similar to NBL4 | na |
| 1373920_at | similar to 106 kDa O-GlcNAc transferase-interacting protein | na |
| 1372378_at | similar to RIKEN cDNA 4930429A22 | na |
| 1375907_at | similar to CGI-18 protein | na |
| 1380001_at | similar to pinin; DNA segment, Chr 12, ERATO Doi 512, | na |
| | expressed | |
| 1388833_at | similar to DNA polymerase epsilon p17 subunit (DNA | na |
| | polymerase epsilon subunit 3) (Chromatin accessibility | |
| | complex 17) (HuCHRAC17) (CHRAC-17) | |
| 1376137_at | similar to evectin-2 | na |
| 1376494_at | similar to DNA polymerase epsilon p17 subunit (DNA | na |
| | polymerase epsilon subunit 3) (Chromatin accessibility | |
| | complex 17) (HuCHRAC17) (CHRAC-17) | |
| 1384331_at | similar to RIKEN cDNA 1700127B04 | na |
| 1373243_at | similar to RIKEN cDNA 1110011E12 | na |
| 1376562_at | similar to misshapen/NIK-related kinase isoform 2; GCK | na |
| | family kinase MINK; serine/threonine protein kinase | |
| 1378218_at | similar to transcription factor CP2; Transcription factor CP2, | na |
| | alpha globin | |
| 1395363_at | similar to methionine-tRNA synthetase; methionine tRNA | na |
| | ligase; methionyl-tRNA synthetase | |
| 1391919_at | similar to hypothetical protein LOC256536 | na |
| 1383912_at | similar to headcase homolog; hHDC for homolog of Drosophila | na |
| | headcase | |
| 1372920_at | similar to proline dehydrogenase; PRODH | na |
| 1375193_at | similar to Kunitz-type protease inhibitor 1 precursor | na |
| | (Hepatocyte growth factor activator inhibitor type 1) (HAI-1) | |
| 1372377_at | similar to DEAD-box protein abstrakt homolog | na |
| 1368895_at | neuroligin 2 | Nlgn2 |
| 1387929_at | PMF32 protein | Pmf31 |
| 1371869_at | proteasome (prosome, macropain) subunit, alpha type 7 | Psma7 |
| 1367851_at | prostaglandin D2 synthase | Ptgds |
| 1370806_at | all-trans-13,14-dihydroretinol saturase | Rmt7 |
| 1393731_at | ring finger protein 40 | Rnf40 |
| 1370883_at | RT1 class II, locus Da | RT1-Da |
| 1391946_at | selectin, platelet | Selp |
| 1367802_at | serum/glucocorticoid regulated kinase | Sgk |
| 1368440_at | solute carrier family 3, member 1 | Slc3a1 |
| 1388831_at | solute carrier family 9 (sodium/hydrogen exchanger), isoform 3 | Slc9a3r2 |
| | regulator 2 | |
| 1368104_at | tetraspan 2 | Tspan2 |
| 1367598_at | transthyretin | Ttr |
| 1369705_at | X transporter protein 3 | Xtrp3 |

Genes and gene fragments identified as changing significantly in animals 6 hours following passive avoidance training relative to controls are provided in Table 15 below.

TABLE 15

| Fragment Name | Gene Name | Gene Symbol |
|---|---|---|
| 1369122_at | (Bcl2-associated X protein, similar to bcl2-associated X | (Bax, na) |
| | protein) | |
| 1388947_at | (eukaryotic translation initiation factor 5B, similar to | (Eif5b, na) |
| | Translation initiation factor IF-2) | |
| 1389088_at | activity-dependent neuroprotective protein | Adnp |
| 1388237_s_at | ATPase, Ca++ transporting, plasma membrane 3 | Atp2b3 |
| 1370528_at | casein kinase 1, delta | Csnk1d |
| 1370123_a_at | cortactin isoform B | Cttn |
| 1385517_at | general transcription factor II I | Gtf2i |
| 1371442_at | hypoxia up-regulated 1 | Hyou1 |
| 1387652_at | insulin degrading enzyme | Ide |
| 1387394_at | interleukin 2 receptor, beta chain | Il2rb |
| 1367796_at | N-acetylglucosaminyltransferase I | Mgat1 |
| 1388699_at | similar to mannosidase 2, alpha B1 | MGC72561 |
| 1376336_at | similar to RNA polymerase II transcriptional regulation | na |
| | mediator (Med6, S. cerevisiae, homolog of) | |
| 1391462_at | similar to emopamil binding related protein EBRP | na |
| 1379304_at | similar to hypothetical protein FLJ13902 | na |
| 1374078_at | similar to BRAF35/HDAC2 complex | na |
| 1391855_at | similar to B-cell CLL/lymphoma 7A | na |
| 1392705_at | similar to RIKEN cDNA 6430520C02 | na |
| 1383336_at | similar to pinin; DNA segment, Chr 12, ERATO Doi 512, | na |
| | expressed | |
| 1382306_at | similar to Ariadne-1 protein homolog (ARI-1) (Ubiquitin- | na |
| | conjugating enzyme E2-binding protein 1) (UbcH7-binding | |
| | protein) (UbcM4-interacting protein) (HHARI) (H7-AP2) | |
| | (HUSSY-27) (MOP-6) | |
| 1376961_at | similar to mKIAA1246 protein | na |
| 1375907_at | similar to CGI-18 protein | na |
| 1374831_at | similar to cDNA sequence BC023151 | na |
| 1381009_at | similar to KIAA0789 protein | na |
| 1374983_at | similar to cDNA sequence BC012312 | na |
| 1373980_at | similar to RIKEN cDNA 2410004J02 | na |
| 1379664_at | similar to KIAA0853 protein | na |
| 1376243_at | similar to RIKEN cDNA 2300003F07 | na |
| 1373737_at | similar to hypothetical protein | na |
| 1372620_at | similar to acidic (leucine-rich) nuclear phosphoprotein 32 | na |
| | family, member E; leucine-rich acidic nuclear protein like | |
| 1391658_at | similar to archain 1 | na |
| 1376645_at | similar to RIKEN cDNA 6330406I15 | na |
| 1376516_at | similar to RIKEN cDNA E030034P13 | na |
| 1382041_at | similar to 1-acylglycerol-3-phosphate-gamma | na |
| 1384342_at | nischarin | Nisch |
| 1387091_at | peptidyl arginine deiminase, type II | Padi2 |
| 1387213_at | proprotein convertase subtilisin/kexin type 4 | Pcsk4 |
| 1367926_at | prohibitin | Phb |
| 1378030_at | phosphatidylinositol binding clathrin assembly protein | Picalm |
| 1388220_at | POU domain, class 2, transcription factor 3 | Pou2f3 |
| 1393480_at | protein phosphatase 1, regulatory (inhibitor) subunit 2 | Ppp1r2 |
| 1373471_at | ring finger protein 166 | Rnf166 |
| 1382055_at | rhotekin | Rtkn |
| 1368372_at | steroid sulfatase | Sts |
| 1388718_at | tropomodulin 1 | Tmod1 |
| 1369164_a_at | transient receptor potential cation channel, subfamily C, | Trpc4 |
| | member 4 | |
| 1369330_at | unc-13 homolog A (C. elegans) | Unc13a |
| 1370343_at | XPA binding protein 2 | Xab2 |

Genes and gene fragments identified as changing significantly in animals 12 hours following passive avoidance training relative to controls are provided in Table 16 below.

TABLE 16

| Fragment Name | Gene Name | Gene Symbol |
|---|---|---|
| 1381509_at | (ovarian carcinoma antigen CA125, similar to Nbr1) | (Ca125, na) |
| 1370948_a_at | (myristoylated alanine rich protein kinase C substrate, similar to | (Marcks, na) |
| | Myristoylated alanine-rich C-kinase substrate (MARCKS)) | |
| 1370113_at | inhibitor of apoptosis protein 1 | Birc3 |
| 1387009_at | calpain 1 | Capn1 |
| 1383985_at | cadherin 15 | Cdh15 |
| 1368636_at | cytochrome P450, family 27, subfamily b, polypeptide 1 | Cyp27b1 |
| 1392196_at | discs, large (Drosophila) homolog 2 (chapsyn-110) | Dlg2 |
| 1397587_at | glucose 6 phosphatase, catalytic, 3 | G6pc3 |
| 1391805_at | similar to KIAA1900 protein | na |
| 1384480_at | similar to RIKEN cDNA 1810048J11 | na |
| 1371999_at | similar to dJ862K6.2.2 (splicing factor, arginine/serine-rich 6 | na |
| | (SRP55-2)(isoform 2)) | |
| 1396082_at | similar to BTB and kelch domain containing 3 | na |
| 1372902_at | similar to putative mannosyltransferase | na |
| 1374921_at | similar to helicase-like protein NHL isoform 2 | na |
| 1395761_at | similar to semaphorin A | na |
| 1389551_at | similar to Lactamase, beta 2 | na |
| 1368820_at | nuclear transcription factor-Y gamma | Nfyc |
| 1397797_at | tigger transposable element derived 3 homolog | Tigd3 |

Genes and gene fragments identified as changing significantly in animals 24 hours following passive avoidance training relative to controls are provided in Table 17 below.

TABLE 17

| Fragment Name | Gene Name | Gene Symbol |
|---|---|---|
| 1391538_at | (apoptotic protease activating factor 1, cysteine-sulfinate | (Apaf1, Csad) |
| | decarboxylase) | |
| 1370913_at | (Best5 protein, hypothetical gene supported by NM_138881) | (Best5, na) |
| 1369937_at | (calmodulin 1, hypothetical gene supported by NM_031969; | (Calm1, na) |
| | AF178845) | |
| 1382913_at | (brain specific cortactin-binding protein CBP90, similar to | (Cbp90, na) |
| | cortactin binding protein 2; cortactin-binding protein 2; | |
| | chromosome 7 open reading frame 8) | |
| 1397200_at | (chromodomain helicase DNA binding protein 4, similar to | (Chd4, na) |
| | chromodomain helicase DNA binding protein 4; Mi-2b) | |
| 1367818_at | (coenzyme Q3 homolog, methyltransferase (yeast), hypothetical | (Coq3, na) |
| | gene supported by NM_019187) | |
| 1375127_at | (cytochrome c oxidase, subunit Va, similar to CYTOCHROME | (Cox5a, na) |
| | C OXIDASE POLYPEPTIDE VA, MITOCHONDRIAL | |
| | PRECURSOR) | |
| 1386921_at | (carboxypeptidase E, similar to Carboxypeptidase H precursor | (Cpe, na) |
| | (CPH) (Carboxypeptidase E) (CPE) (Enkephalin convertase) | |
| | (Prohormone processing carboxypeptidase)) | |
| 1395925_s_at | (BMP/retinoic acid-inducible neural-specific protein, discs, | (Dlgh3, LOC140610) |
| | large homolog 3 (Drosophila)) | |
| 1367565_a_at | (ferritin, heavy polypeptide 1, similar to ferritin, heavy | (Fth1, na) |
| | polypeptide 1; Ferritin subunit H) | |
| 1371245_a_at | (hemoglobin beta chain complex, similar to Hemoglobin beta | (Hbb, na) |
| | chain, minor-form) | |
| 1398240_at | (heat shock protein 8, similar to Heat shock cognate 71 kDa | (Hspa8, na) |
| | protein) | |
| 1387770_at | (interferon, alpha-inducible protein 27-like, putative ISG12(a) | (Ifi271, isg12(a)) |
| | protein) | |
| 1388152_at | (hypothetical gene supported by NM_013066, microtubule- | (Mtap2, na) |
| | associated protein 2) | |
| 1387571_at | (hypothetical gene supported by NM_031130, nuclear receptor | (Nr2f1, na) |
| | subfamily 2, group F, member 1) | |
| 1368993_at | (hypothetical gene supported by NM_020088, odd Oz/ten-m | (Odz2, na) |
| | homolog 2 (Drosophila)) | |
| 1368508_at | (proteasome (prosome, macropain) subunit, alpha type 3, | (Psma3, Psma3l) |
| | proteasome subunit alpha type 3-like) | |
| 1370428_x_at | (RT1 class I, A3, RT1 class Ib, locus Aw2, RT1 class Ib, locus | (RT1-A3, RT1-Aw2, RT1-Cl) |
| | Cl) | |
| 1370188_at | (similar to splicing factor, arginine/serine-rich (transformer 2 | (Sfrs10, na) |
| | Drosophila homolog) 10, splicing factor, arginine/serine-rich 10 | |
| | (transformer 2 homolog, Drosophila)) | |
| 1368098_a_at | (SNRPN upstream reading frame, similar to small nuclear | (Snrpn, Snurf, na) |
| | ribonucleoprotein-associated protein N - rat, small nuclear | |
| | ribonucleoprotein N) | |
| 1369085_s_at | (SNRPN upstream reading frame, similar to small nuclear | (Snrpn, Snurf, na) |
| | ribonucleoprotein-associated protein N - rat, small nuclear | |
| | ribonucleoprotein N) | |
| 1370807_at | (similar to vacuole Membrane Protein 1, vacuole Membrane | (Vmp1, na) |
| | Protein 1) | |
| 1387862_at | (similar to 14-3-3 PROTEIN TAU (14-3-3 PROTEIN THETA), | (Ywhaq, na) |
| | similar to 14-3-3 protein tau (14-3-3 protein theta) (14-3-3 | |
| | protein T-cell) (HS1 protein), tyrosine 3- | |
| | monooxygenase/tryptophan 5-monooxygenase activation | |
| | protein, theta polypeptide) | |
| 1372552_at | DMT1-associated protein | Acbd3 |
| 1397375_at | acyl-CoA synthetase long-chain family member 5 | Acsl5 |
| 1398835_at | actin, beta | Actb |
| 1398836_s_at | actin, beta | Actb |
| 1387811_at | angiotensinogen | Agt |
| 1387022_at | aldehyde dehydrogenase family 1, member A1 | Aldh1a1 |
| 1368003_at | aldehyde dehydrogenase family 1, subfamily A2 | Aldh1a2 |
| 1369063_at | acidic (leucine-rich) nuclear phosphoprotein 32 family, member A | Anp32a |
| 1370862_at | apolipoprotein E | Apoe |
| 1380533_at | amyloid beta (A4) precursor protein | App |
| 1371571_at | amyloid beta (A4) precursor protein | App |
| 1387651_at | aquaporin 1 | Aqp1 |
| 1368317_at | aquaporin 7 | Aqp7 |
| 1388997_at | ADP-ribosylation factor 3 | Arf3 |
| 1387447_at | ADP-ribosylation factor 3 | Arf3 |
| 1369998_at | ADP-ribosylation factor 6 | Arf6 |
| 1370275_at | ATP synthase, H+ transporting, mitochondrial F1 complex, beta | Atp5b |
| | polypeptide | |
| 1398755_at | ATPase, H+ transporting, lysosomal (vacuolar proton pump) 16 kDa | Atp6l |
| 1387184_at | axin2 | Axin2 |
| 1386991_a_at | bcl-2 associated death agonist | Bad |
| 1398350_at | brain abundant, membrane attached signal protein 1 | Basp1 |
| 1372710_at | blocked early in transport 1 homolog (S. cerevisiae) | Bet1 |
| 1392077_at | chromosome 11 open reading frame 8 | C11orf8 |
| 1377640_at | calcitonin receptor-like | Calcrl |
| 1370246_at | calmodulin 2 | Calm2 |
| 1370873_at | calmodulin 3 | Calm3 |
| 1368101_at | calmodulin 3 | Calm3 |
| 1388187_at | calcium/calmodulin-dependent protein kinase II alpha subunit | Camk2a |
| 1399033_at | core binding factor beta | Cbfb |
| 1371953_at | cyclin G2 | Ccng2 |
| 1395508_at | chaperonin containing TCP1, subunit 5 (epsilon) | Cct5 |
| 1367784_a_at | clusterin | Clu |
| 1389891_at | procollagen, type XI, alpha 2 | Col11a2 |
| 1372801_at | COMM domain containing 10 | Commd10 |
| 1367454_at | coatomer protein complex, subunit beta 2 (beta prime) | Copb2 |
| 1398357_at | complexin 1 | Cplx1 |
| 1368849_at | casein kinase 1, gamma 3 | Csnk1g3 |
| 1370855_at | cystatin C | Cst3 |
| 1387720_at | calsyntenin 2 | Cstn2 |
| 1387880_at | CUG triplet repeat, RNA-binding protein 2 | Cugbp2 |
| 1384392_at | cytochrome P450, family 26, subfamily b, polypeptide 1 | Cyp26b1 |
| 1390738_at | DAMP-1 protein | Damp1 |
| 1398795_at | aspartyl-tRNA synthetase | Dars |
| 1382348_at | dihydroorotate dehydrogenase | Dhodh |
| 1370328_at | dickkopf homolog 3 (Xenopus laevis) | Dkk3 |
| 1387274_at | distal-less homeobox 5 | Dlx5 |
| 1368292_at | dynamin 1 | Dnm1 |
| 1371694_at | dihydropyrimidinase-like 2 | Dpysl2 |
| 1375357_at | dystonia 1, torsion (autosomal dominant; torsin A) | Dyt1 |
| 1369519_at | endothelin 1 | Edn1 |
| 1371527_at | epithelial membrane protein 1 | Emp1 |
| 1368536_at | ectonucleotide pyrophosphatase/phosphodiesterase 2 | Enpp2 |
| 1388358_at | electron-transfer-flavoprotein, beta polypeptide | Etfb |
| 1368114_at | fibroblast growth factor 13 | Fgf13 |
| 1371179_a_at | fibroblast growth factor receptor 2 | Fgfr2 |
| 1368693_at | FGR | Fgr |
| 1373757_at | FLN29 gene product | Fln29 |
| 1367700_at | fibromodulin | Fmod |
| 1387889_at | folate receptor 1 (adult) | Folr1 |
| 1378239_at | glucosidase, alpha; acid (Pompe disease, glycogen storage | Gaa |
| | disease type II) | |
| 1369371_a_at | gamma-aminobutyric acid (GABA) B receptor, 1 | Gabbr1 |
| 1369048_at | gamma-aminobutyric acid A receptor, delta | Gabrd |
| 1383047_at | growth arrest specific 6 | Gas6 |
| 1373386_at | gap junction membrane channel protein beta 2 | Gjb2 |
| 1367633_at | glutamine synthetase 1 | Glu1 |
| 1369897_s_at | GNAS complex locus | Gnas |
| 1374062_x_at | G protein-coupled receptor kinase 5 | Gprk5 |
| 1370813_at | glutathione S-transferase, mu 5 | Gstm5 |
| 1387775_at | general transcription factor IIa, 2 (12 kD subunit) | Gtf2a2 |
| 1369319_at | glutamate transporter EAAC1 interacting protein | Gtrap3-18 |
| 1369097_s_at | guanylate cyclase 1, soluble, beta 3 | Gucy1b3 |
| 1388608_x_at | hemoglobin alpha, adult chain 1 | Hba-a1 |
| 1395561_at | heterogeneous nuclear ribonucleoprotein A2/B1 | Hnrpa2b1 |
| 1387387_at | hippocalcin | Hpca |
| 1389384_at | hormone-regulated proliferation-associated 20 kDa protein | HRPAP20 |
| 1372701_at | heat shock protein 1, alpha | Hspca |
| 1375336_at | heat shock 90 kDa protein 1, beta | Hspcb |
| 1375335_at | heat shock 90 kDa protein 1, beta | Hspcb |
| 1369860_a_at | 5-hydroxytryptamine (serotonin) receptor 2C | Htr2c |
| 1370665_at | hypoxia up-regulated 1 | Hyou1 |
| 1381350_at | inhibitor of DNA binding 4 | Idb4 |
| 1394022_at | inhibitor of DNA binding 4 | Idb4 |
| 1371186_at | integrin, alpha 6 | Itga6 |
| 1374404_at | v-jun sarcoma virus 17 oncogene homolog (avian) | Jun |
| 1370773_a_at | Kv channel-interacting protein 2 | Kcnip2 |
| 1375190_at | potassium channel tetramerisation domain containing 13 | Kctd13 |
| 1387327_at | KH domain containing, RNA binding, signal transduction | Khdrbs2 |
| | associated 2 | |
| 1391906_at | kinesin family member 1B | Kif1b |
| 1389157_at | lectin, galactoside-binding, soluble, 2 (galectin 2) | Lgals2 |
| 1387946_at | lectin, galactoside-binding, soluble, 3 binding protein | Lgals3bp |
| 1381190_at | LIM domain only protein 7 | LMO7 |
| 1370358_at | voltage-gated Ca channel | LOC246215 |
| 1370442_at | thymosin beta-like protein | LOC286978 |
| 1370853_at | CaM-kinase II inhibitor alpha | LOC287005 |
| 1384878_at | N-myristoyltransferase 2 | LOC291318 |
| 1393260_at | WDNM1 homolog | LOC360228 |
| 1397415_at | atlastin-like | LOC362750 |
| 1368700_at | 130 kDa-Ins(1, 4, 5)P3 binding protein | LOC84587 |
| 1395316_at | melanoma antigen, family H, 1 | Mageh1 |
| 1387920_at | mannosidase, alpha, class 2C, member 1 | Man2c1 |
| 1377831_at | monoamine oxidase A | Maoa |
| 1376525_at | MAP2 RNA trans-acting protein MARTA1 | Marta1 |
| 1367682_at | midkine | Mdk |
| 1372457_at | mitochondrial tumor suppressor 1 | Mtus1 |
| 1387283_at | myxovirus (influenza virus) resistance 2 | Mx2 |
| 1370158_at | myosin heavy chain 10, non-muscle | Myh10 |
| 1368450_at | myosin Va | Myo5a |
| 1374058_at | similar to D7H11orf15 protein | na |
| 1377434_at | similar to membrane-type frizzled-related protein | na |
| 1383448_at | similar to interferon stimulated gene factor 3 gamma | na |
| 1382561_at | similar to 4921517L17Rik protein | na |
| 1371320_at | similar to E25B protein | na |
| 1371334_at | similar to integral membrane protein 2C | na |
| 1371393_at | similar to calsyntenin-1 protein | na |
| 1383564_at | similar to interferon regulatory factor 7 | na |
| 1380682_at | similar to KIAA2009 protein | na |
| 1371474_at | similar to Mtch1 protein | na |
| 1377615_at | similar to hypothetical protein | na |
| 1388576_at | similar to D5Wsu45e protein | na |
| 1382064_at | similar to 4933407C03Rik protein | na |
| 1389251_at | similar to coenzyme A diphosphatase | na |
| 1383339_at | similar to NNX3 | na |
| 1373940_at | similar to non-catalytic region of tyrosine kinase adaptor | na |
| | protein 1 | |
| 1372003_at | similar to CG17660-PA | na |
| 1371544_at | similar to Enhancer of rudimentary homolog | na |
| 1392157_at | similar to plexin 2 | na |
| 1377993_at | similar to C321D2.5 (novel protein) | na |
| 1371853_at | similar to RIKEN cDNA 2900055D03 | na |
| 1389636_at | similar to KIAA0833 protein | na |
| 1388428_at | similar to histidyl tRNA synthetase 2 | na |
| 1376676_a_at | similar to Ab2-008 | na |
| 1395699_at | similar to sudD, suppressor of bimD6 homolog | na |
| 1388341_at | similar to hypothetical protein | na |
| 1381014_at | similar to RIKEN cDNA A430056A10; expressed sequence | na |
| | AW261460 | |
| 1389885_at | similar to RIKEN cDNA 0610025L06 | na |
| 1377937_at | similar to mitochondrial ribosomal protein S14 | na |
| 1373615_at | similar to frezzled | na |
| 1376630_at | similar to RIKEN cDNA A530046H20 | na |
| 1376805_at | similar to ring finger protein 2 | na |
| 1384437_at | similar to SWI/SNF-related matrix-associated actin-dependent | na |
| | regulator of chromatin a1 isoform a; sucrose nonfermenting 2- | |
| | like protein 1; SNF2-like 1; global transcription activator | |
| | homologous sequence | |
| 1379401_a_at | similar to 5730501N20Rik protein | na |
| 1375056_at | similar to hypothetical protein MGC45416 | na |
| 1372187_at | similar to RIKEN cDNA 4930557O20 | na |
| 1391117_at | similar to KIAA1183 protein | na |
| 1371909_at | similar to Cofactor required for Sp1 transcriptional activation | na |
| | subunit 3 (Transcriptional co-activator CRSP130) (Vitamin D3 | |
| | receptor-interacting protein complex 130 kDa component) | |
| | (DRIP130) (Activator-recruited cofactor 130 kDa component) | |
| | (ARC130)... | |
| 1374627_at | similar to interferon stimulated gene factor 3 gamma | na |
| 1388766_at | similar to metaxin 2 | na |
| 1389975_at | similar to HUD3 | na |
| 1391158_a_at | similar to ataxin-2 | na |
| 1374215_at | similar to hypothetical protein FLJ10297 | na |
| 1384983_at | similar to RIKEN cDNA 1110032A17 | na |
| 1374882_at | similar to HT021 | na |
| 1367494_at | similar to CGI-35 protein | na |
| 1373011_at | (similar to Ac1133, similar to putative Zn-finger protein C47S) | na |
| 1371378_at | (similar to Chain, Human Translation Initiation Factor Eif1, | na |
| | Nmr, 29 Structures, similar to Protein translation factor SUI1 | |
| | homolog) | |
| 1379803_at | similar to LIM domain only 4; LIM only 4; ethanol induced 4 | na |
| 1376336_at | similar to RNA polymerase II transcriptional regulation | na |
| | mediator (Med6, S. cerevisiae, homolog of) | |
| 1379206_at | similar to RIKEN cDNA 1110001E17 | na |
| 1392045_at | similar to hypothetical protein MGC3295 | na |
| 1382061_at | similar to Ac2-202 | na |
| 1388847_at | similar to Pcqap protein | na |
| 1384172_at | similar to KIAA0423 | na |
| 1384464_at | similar to SREBP cleavage activating protein | na |
| 1373547_at | similar to expressed sequence AW209491 | na |
| 1376339_at | similar to KIAA1280 protein | na |
| 1394414_at | similar to RIKEN cDNA 2310034L04 | na |
| 1376298_at | similar to UPF3 regulator of nonsense transcripts homolog B | na |
| | isoform 1 | |
| 1377252_at | similar to RIKEN cDNA 1200011M11 | na |
| 1373199_at | similar to large subunit ribosomal protein L36a | na |
| 1371523_at | similar to MURR1 | na |
| 1395274_at | similar to bullous pemphigoid antigen 1-b | na |
| 1372618_at | similar to hypothetical protein FLJ13045 | na |
| 1378445_at | LOC361197 | na |
| 1375140_at | similar to muscleblind-like protein 2 isoform 1; muscleblind- | na |
| | like protein MBLL39 | |
| 1391754_at | similar to 25 oligoadenylate synthetase | na |
| 1393340_at | similar to MondoA | na |
| 1375459_at | hypothetical gene supported by AF102149 | na |
| 1373748_at | similar to semaphorin cytoplasmic domain-associated protein | na |
| | 3A | |
| 1388416_at | similar to lipoprotein receptor-related protein | na |
| 1374644_at | hypothetical LOC306165 | na |
| 1373756_at | similar to BM88 antigen | na |
| 1379781_at | similar to BAF53a | na |
| 1383138_at | similar to slingshot 3 | na |
| 1371891_at | similar to 1700123O20Rik protein | na |
| 1376274_at | similar to RIKEN cDNA 1110056N09 | na |
| 1384480_at | similar to RIKEN cDNA 1810048J11 | na |
| 1371634_at | similar to RIKEN cDNA 1810020E01 | na |
| 1390851_at | similar to Lactamase, beta 2 | na |
| 1372686_at | hypothetical LOC291080 | na |
| 1377263_at | similar to cofactor required for Sp1 transcriptional activation, | na |
| | subunit 9, 33 kDa | |
| 1372034_at | similar to hypothetical protein MGC29390 | na |
| 1397201_at | similar to KIAA1078 protein | na |
| 1388310_at | (similar to Chain, Human Translation Initiation Factor Eif1, | na |
| | Nmr, 29 Structures, similar to Protein translation factor SUI1 | |
| | homolog) | |
| 1375961_at | similar to frezzled | na |
| 1374061_at | similar to RIKEN cDNA 1110055L24 | na |
| 1380867_a_at | similar to hypothetical protein LOC201175 | na |
| 1395612_at | similar to pellino 3 alpha | na |
| 1391475_at | similar to 2810036L13Rik protein | na |
| 1383118_at | similar to hypothetical protein FLJ14803 | na |
| 1398664_at | similar to RIKEN cDNA 9130427A09 | na |
| 1393231_at | hypothetical LOC297486 | na |
| 1373437_at | similar to RIKEN cDNA 6330548N22 | na |
| 1373450_at | similar to 4833420O05Rik protein | na |
| 1393087_at | similar to RecQ helicase protein-like 5 beta | na |
| 1371999_at | similar to dJ862K6.2.2 (splicing factor, arginine/serine-rich 6 | na |
| | (SRP55-2)(isoform 2)) | |
| 1382045_at | similar to hypothetical protein FLJ12085 | na |
| 1377458_at | similar to gp250 precursor | na |
| 1383837_at | similar to DNA segment, Chr 17, Wayne State University 94, | na |
| | expressed | |
| 1388471_at | similar to RIKEN cDNA E430026E19 | na |
| 1381564_at | similar to glomulin, FKBP associated protein; FKBP-associated | na |
| | protein | |
| 1398649_at | similar to neuronal transmembrane protein Slitrk3 | na |
| 1385295_at | similar to hypothetical protein MGC20983 | na |
| 1377341_at | similar to hypothetical protein FLJ10925 | na |
| 1398354_at | similar to alpha-catenin related protein | na |
| 1392506_at | similar to RIKEN cDNA 2210407J23 | na |
| 1398626_s_at | similar to actin-related protein 2 | na |
| 1377375_at | similar to aminoadipate-semialdehyde synthase | na |
| 1375060_at | similar to Cc2-27 | na |
| 1371256_at | similar to protein tyrosine phosphatase 20 | na |
| 1372576_at | similar to expressed sequence C85658 | na |
| 1372905_at | similar to Vinculin (Metavinculin) | na |
| 1372394_at | similar to E3 ligase for inhibin receptor | na |
| 1395371_at | similar to FLJ00052 protein | na |
| 1374571_at | similar to hypothetical protein FLJ20522 | na |
| 1388996_at | similar to RIKEN cDNA 3110052N05 | na |
| 1394968_at | similar to PHD-like zinc finger protein | na |
| 1376447_at | similar to hypothetical protein A430110N23 | na |
| 1372142_at | similar to arsenic resistance ATPase | na |
| 1398578_at | similar to leukocyte receptor cluster (LRC) member 8 | na |
| 1373840_at | similar to nudix (nucleoside diphosphate linked moiety X)-type | na |
| | motif 9 | |
| 1376499_at | similar to MR1-interacting protein | na |
| 1381103_a_at | similar to bK1048E9.3 (novel protein) | na |
| 1390937_at | similar to chromosome 14 open reading frame 50 | na |
| 1372474_at | similar to synaptophysin-like protein; pantophysin; Pan I; DNA | na |
| | segment, Chr 12, ERATO Doi 446, expressed | |
| 1390865_at | similar to Ca2+-dependent activator for secretion protein 2 | na |
| 1374913_at | similar to hypothetical protein D12Ertd771e | na |
| 1374183_at | similar to RIKEN cDNA 1810011O16 | na |
| 1373504_at | similar to GLI pathogenesis-related 1 (glioma); related to testes- | na |
| | specific, vespid, and pathogenesis proteins | |
| 1376001_at | similar to RNA polymerase I associated factor (PAF53) | na |
| 1377665_at | similar to KIAA0404 | na |
| 1372423_at | similar to p53 apoptosis-associated target | na |
| 1383335_at | similar to Link guanine nucleotide exchange factor II | na |
| 1376340_a_at | similar to tumor-suppressing subchromosomal transferable | na |
| | fragment 4 | |
| 1382146_at | similar to transmembrane 4 superfamily member 6 | na |
| 1384290_at | similar to RIKEN cDNA C030034J04 | na |
| 1373835_at | similar to F-box only protein 8 | na |
| 1398912_at | similar to 60S ribosomal protein L9, mitochondrial precursor | na |
| | (L9mt) | |
| 1399000_at | similar to Potential phospholipid-transporting ATPase IIB | na |
| 1376254_at | similar to KIAA1440 protein | na |
| 1388579_at | similar to CG4241-PA | na |
| 1390699_at | similar to KIAA2026 protein | na |
| 1391043_at | similar to C15orf16 protein | na |
| 1379525_at | similar to chromosome 20 open reading frame 155 | na |
| 1398914_at | similar to DNA-directed RNA polymerase II 13.3 kDa | na |
| | polypeptide (RPB11) | |
| 1389386_at | similar to hypothetical protein DKFZp313N0621 | na |
| 1389569_at | similar to BRIX | na |
| 1395519_at | similar to B230212L03Rik protein | na |
| 1374313_at | similar to RIKEN cDNA 6430517J16 | na |
| 1384289_at | hypothetical LOC291630 | na |
| 1373381_at | similar to HECT domain and RCC1-like domain protein 3 | na |
| 1388431_at | similar to SYT | na |
| 1389910_at | similar to chromosome 20 open reading frame 30; HSPC274 | na |
| | protein | |
| 1388321_at | similar to RIKEN cDNA 1190002L16 | na |
| 1376440_at | similar to ring finger 139; patched related protein translocated | na |
| | in renal cancer; ring finger protein 139 | |
| 1395400_at | similar to hypothetical protein MGC23280 | na |
| 1372463_at | similar to hypothetical protein 5832424M12 | na |
| 1390118_at | similar to ATP/GTP-binding protein | na |
| 1375848_at | similar to Pon2 protein | na |
| 1372936_at | similar to Purkinje cell protein 2 - mouse | na |
| 1373064_at | similar to DnaJ (Hsp40) homolog, subfamily D, member 1 | na |
| 1388594_at | similar to cysteine-rich with EGF-like domains 1 | na |
| 1376261_at | similar to Bag5 protein | na |
| 1396478_at | similar to hypothetical protein FLJ22679 | na |
| 1377644_at | similar to RIKEN cDNA 4921524J17 | na |
| 1367956_at | neurochondrin | Ncdn |
| 1370229_at | N-myc downstream regulated 4 | Ndr4 |
| 1386951_at | NADH dehydrogenase (ubiquinone) 1 alpha subcomplex 5 | Ndufa5 |
| 1371518_at | nidogen (entactin) | Nid |
| 1369999_a_at | neuronatin | Nnat |
| 1376362_at | neuronal pentraxin receptor | Nptxr |
| 1370211_at | neurogranin | Nrgn |
| 1370934_at | nucleoporin 153 kD | Nup153 |
| 1371963_at | propionyl-coenzyme A carboxylase, alpha polypeptide | Pcca |
| 1398271_at | piccolo (presynaptic cytomatrix protein) | Pclo |
| 1367671_at | proliferating cell nuclear antigen | Pcna |
| 1367835_at | proprotein convertase subtilisin/kexin type 1 inhibitor | Pcsk1n |
| 1379361_at | peroxisomal biogenesis factor 11A | Pex11a |
| 1368399_a_at | plasma glutamate carboxypeptidase | Pgcp |
| 1370100_at | phosphatidylinositol 3-kinase, regulatory subunit, polypeptide 2 | Pik3r2 |
| 1371203_at | protein kinase C, lambda | Pkcl |
| 1369105_a_at | protein kinase (cAMP dependent, catalytic) inhibitor beta | Pkib |
| 1383191_at | protein phosphatase 1, regulatory (inhibitor) subunit 1A | Ppp1r1a |
| 1379374_at | plasticity related gene 1 | Prg1 |
| 1370156_at | prion protein | Prnp |
| 1367710_at | protease (prosome, macropain) 28 subunit, beta | Psme2 |
| 1368421_at | protein tyrosine phosphatase, non-receptor type 5 | Ptpn5 |
| 1368358_a_at | protein tyrosine phosphatase, receptor type, R | Ptprr |
| 1388615_at | Ras-related protein RAP-1A | RAP-1A |
| 1381857_at | RAS p21 protein activator 1 | Rasa1 |
| 1382379_at | ring finger protein 138 | Rnf138 |
| 1369338_at | roundabout homolog 1 (Drosophila) | Robo1 |
| 1368932_at | Rho-associated kinase beta | Rock1 |
| 1373999_at | restin (Reed-Steinberg cell-expressed intermediate filament- | Rsn |
| | associated protein) | |
| 1371123_x_at | MHC class Ib RT1.S3 | RT1.S3 |
| 1388212_a_at | MHC class Ib RT1.S3 | RT1.S3 |
| 1388071_x_at | RT1 class Ib, locus Aw2 | RT1-Aw2 |
| 1368097_a_at | reticulon 1 | Rtn1 |
| 1370981_at | retinoid X receptor gamma | Rxrg |
| 1367668_a_at | stearoyl-Coenzyme A desaturase 2 | Scd2 |
| 1386889_at | stearoyl-Coenzyme A desaturase 2 | Scd2 |
| 1387294_at | SH3-domain binding protein 5 (BTK-associated) | Sh3bp5 |
| 1396464_at | SH3 domain protein 2A | Sh3gl2 |
| 1369732_a_at | sialyltransferase 4B (beta-galactoside alpha-2,3- | Siat4b |
| | sialyltransferase) | |
| 1370803_at | SNAP25 interacting protein 30 | Sip30 |
| 1398255_at | solute carrier family 15 (H+/peptide transporter), member 2 | Slc15a2 |
| 1370286_at | solute carrier family 38, member 2 | Slc38a2 |
| 1368082_at | solute carrier family 4, member 2 | Slc4a2 |
| 1368606_at | solute carrier organic anion transporter family, member 1A2 | Slco1a2 |
| 1375469_at | SWI/SNF related, matrix associated, actin dependent regulator | Smarca4 |
| | of chromatin, subfamily a, member 4 | |
| 1387073_at | synaptosomal-associated protein | Snap25 |
| 1379281_at | uterine sensitization-associated gene 1 protein | Sostdc1 |
| 1369991_at | signal peptidase complex 18 kD | Spc18 |
| 1387354_at | signal transducer and activator of transcription 1 | Stat1 |
| 1372757_at | signal transducer and activator of transcription 1 | Stat1 |
| 1370614_s_at | serine threonine kinase 39 (STE20/SPS1 homolog, yeast) | Stk39 |
| 1387359_at | syntaxin 1A (brain) | Stx1a |
| 1370518_a_at | syntaxin binding protein 1 | Stxbp1 |
| 1369627_at | synaptic vesicle glycoprotein 2 b | Sv2b |
| 1369628_at | synaptic vesicle glycoprotein 2 b | Sv2b |
| 1369482_a_at | synapsin II | Syn2 |
| 1373896_at | synaptotagmin 1 | Syt1 |
| 1369106_at | transcription elongation factor A (SII), 2 | Tcea2 |
| 1387013_at | kidney-specific membrane protein | Tmem27 |
| 1387883_a_at | thymosin beta-4 | Tmsb4x |
| 1379186_at | TOM70 protein | TOM70 |
| 1371241_x_at | tropomyosin 1, alpha | Tpm1 |
| 1367598_at | transthyretin | Ttr |
| 1368994_a_at | tuberin-like protein 1 | Tulip1 |
| 1370274_at | polyubiquitin | Ubb |
| 1386852_x_at | polyubiquitin | Ubb |
| 1390858_at | valosin-containing protein (p97)/p47 complex-interacting | Vcip135 |
| | protein p135 | |
| 1369344_at | WD repeat domain 7 | Wdr7 |
| 1389090_at | Werner syndrome homolog (human) interacting protein | Wrnip |
| 1386866_at | tyrosine 3-monooxgenase/tryptophan 5-monooxgenase | Ywhag |
| | activation protein, gamma polypeptide | |
| 1381998_at | zinc finger protein 148 | Znf148 |

### Example 2

### PCR Confirmation of Mdk Regulation

This example provides confirmation of the pattern of Mdk regulation over time following the learning tasks, using quantitative real-time PCR as described below.

Quantitative Real-Time PCR

Real-time PCR was carried out using TaqMan technology on an ABI Prism 7900HT Sequence Detection System (PE Applied Biosystems, UK). cDNAs, from 1 μg of DNase treated RNA from each animal (n=6 per group) were produced using SuperScript II RNase H Reverse Transcriptase Kit (Invitrogen) and 50-250 ng random primers (Invitrogen). cDNA (0.8 μl) from each sample was amplified using TaqMan® Gene Expression Assay primers and probe (Applied Biosystems, UK), Assay ID Rn_{—}00578324_m1. Relative quantitation was determined by constructing a standard curve for each primer and probe set, using pooled DNA from all the samples. A ribosomal RNA control primer and probe set (Applied Biosystems) was used for normalization purposes.

Preparation of RNA Probes

Riboprobes were prepared from Midkine cDNA (GenBank database accession number NM_{—}030859) by PCR amplification of a 387 by fragment (nucleotides 93-490) using a SP6 (sequence in bold) tagged forward primer 5′-GATTTAGGTGACACTATAGAAGTTTCTTCCTTCTAGCCCTTG-3′ and a T7 (sequence in bold) tagged reverse primer 5′-GTAATACGACTCACTATAGGGTCAGTCCTTTCCTTTTCCTTTC-3′. RNA probes were prepared and labeled with digoxigenin-UTP by in vitro transcription with SP6 and T7 RNA polymerase enzymes [digoxigenin RNA labeling kit (SP6/T7)] according to manufacturer's recommendations (Boehringer Mannheim, Mannheim, Germany).

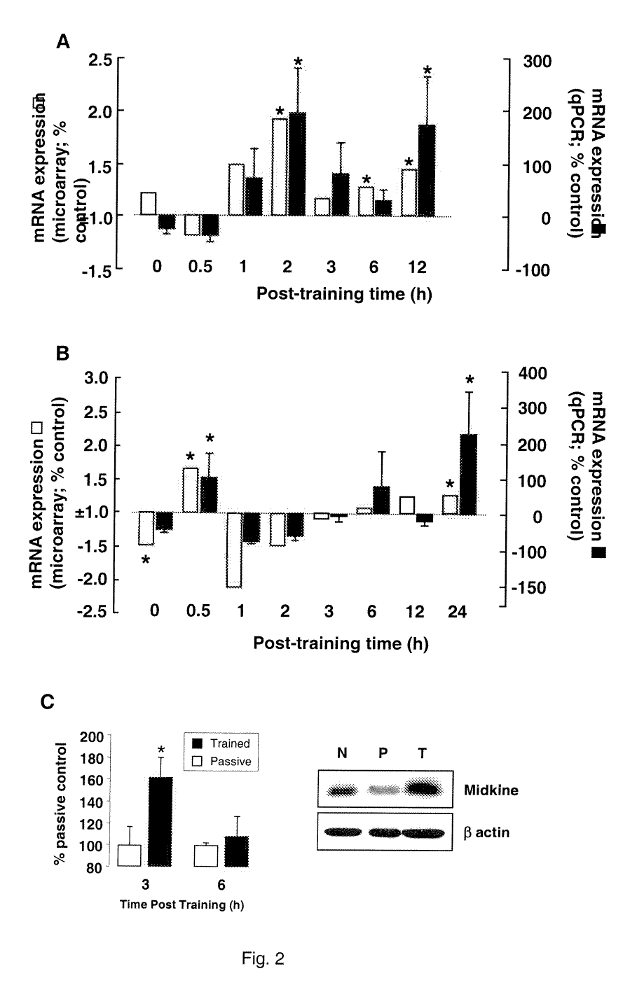

The temporal pattern of mRNA expression of Mdk was confirmed at the time points of interest following water maze training (FIG. 2A), showing an increase at 2 and 24 hours. The temporal pattern of mRNA expression of Mdk was also confirmed at the time points of interest following passive avoidance learning (FIG. 2B), showing an increase at 30 minutes, a decrease at 1 hour, and returning to basal levels thereafter.

### Example 3

### Protein Level Regulation of Mdk

The regulation of Mdk at message level was shown to translate into corresponding protein level regulation following passive avoidance training (FIG. 2C). Protein samples were prepared and analyzed as described below.

Protein Sample Preparation

The dentate gyrus (n=6) from trained and passive animals was homogenised in ice-cold 0.32M sucrose. Protein concentrations were determined according to the method of Bradford (1976). Samples, of equal protein concentrations, were prepared in reducing sample buffer [3X Blue loading buffer with 10% (v/v) dithiothreithol (DTT) (New England Biolabs)] and boiled at 100° C.

SDS-PAGE and Immunoblotting

Normalised proteins samples were separated on polyacrylamide minigels and electrophoretically transferred to nitrocellulose membranes (Bio-rad). Equal protein loading was confirmed by ponceau S staining of the membrane (not shown). The nitrocellulose was blocked in 5% non-fat milk in 10 mM Tris-HCl, 150 nM NaCl, and 0.05% (vol/vol) Tween-20 (TBS-T) for 1 h at room temperature. The monoclonal antibody to midkine (Abeam) was diluted in 5% milk and incubated overnight at 4° C. The membranes were then incubated with appropriate secondary horseradish peroxidase-linked antibodies and visualised by SuperSignal Chemiluminescent Substrate (Pierce) and exposed to X-ray film (Kodak) for appropriate times. Films were digitised and quantitative densitometry was performed using NIH Image software (Version 1.61) to determine amount of midkine protein electrophoresed per sample.

2-Dimensional Gel Electrophoresis

Protein samples were sonicated in lysis buffer (9.5 M urea, 2% CHAPS, 20 mM Tris pH 8.0). CyDye DIGE Fluors minimal labelling (GE Healthcare) that covalently bind to the ε-amino group of lysine of proteins were employed. The internal standard containing an equal amount of protein of all the samples was labelled with Cy2 and was run in all the gels in order to compensate for variation across the gels. After the labelling, 2× dilution buffer (9.5 M urea, 2% CHAPS, 2% DTT, 1.6% Pharmalyte) was added in order to unfold the protein. Strips used were Amersham Bioscience 24 cm strips pH range 4-7; the pool sample, a trained (Cy3 or Cy5) and passive (Cy5 or Cy3) sample were loaded on the same strip overnight during the dehydration step and run the day after on IPGphor (80 kVh). The second dimension was performed in 12% Acrylamide SDS-gel and the gels were scanned on the Typhoon 9410 scanner. Images were analyzed using Decyder 6.5 (GE Healthcare) in BVA mode. Prep gel was ran to cut spots in order to obtain protein ids by mass spectrometry. 300 μg of protein samples were loaded per gel and gels were stained with PlusOne silver staining kit (GE Healthcare).

### Example 4

### Intracerebroventricular Administration of Mdk Improves Memory Consolidation

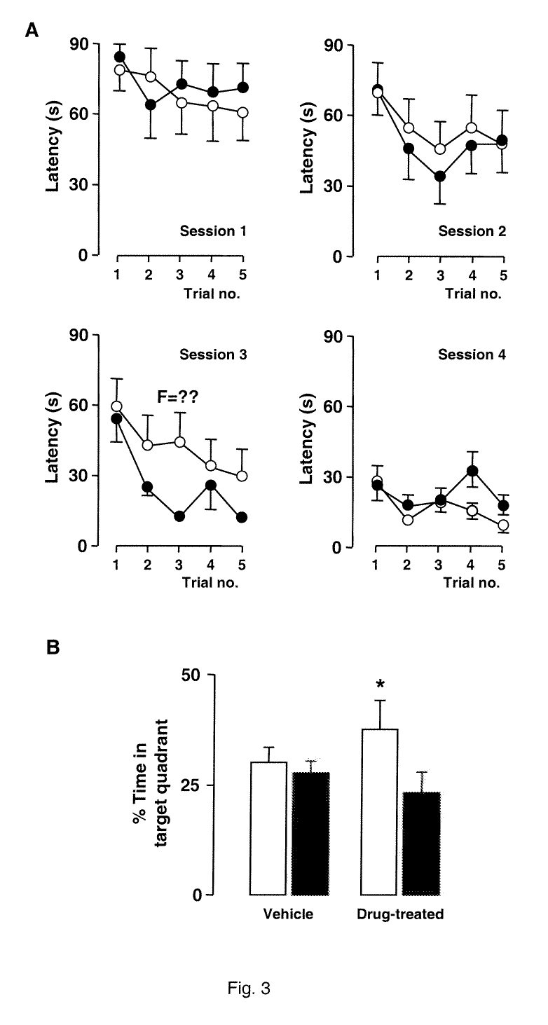

When recombinant Mdk was administered into the 3rd cerebral ventricles of rats, spatial memory was enhanced following training on the Morris Water Maze. Animals were trained on the Morris Water Maze as described above. Mdk improved spatial memory on this learning task when administered to the 3rd cerebral ventricle 2 hours post training on each of the four days of training (FIG. 3A). In addition, Mdk-treated animals demonstrated superior memory of the platform location when tested 24 hours after the fourth and final training session (FIG. 3B).

Mdk administered into the 3rd cerebral ventricles of rats also enhanced recall of an odor reward association. Odor reward association was carried out as described below.

Odor-Reward Association Paradigm

The training protocol employed has been described in detail previously (Foley et al., 2003). The training apparatus was a square box constructed of opaque plastic measuring 34×34×27 cm. Sponges measuring 6×7×2 cm deep had a hole of 2 cm diameter cut into the center and were placed in glass slide-holders of the same size. The food reinforcement was placed at the bottom of the opening in the sponge so the rat had to put its head inside the hole (nose poke) to obtain the chocolate rice crispy breakfast cereal reward (Chocokrispies®, Kellog's, France). On the first trial, four Chocokrispies were also placed on the corners of that sponge, which was impregnated with the target odor, as well as in the hole. The sponge with the non-targeted odor did not contain reinforcement. Sponges were placed in three corners of the box and the position of each odor within the box was changed for each trial according to a previously determined protocol. In addition, the set of sponges was changed between trials to preclude identification based on visual cues. Sponges were impregnated with an odor by placing 15 μl of essence on each corner of the sponge. Odors used were coffee, lime and almond. A video camera was fixed above the apparatus and the rat was observed on a video monitor in the same room. The sessions were recorded on a video tape for possible re-analyses off-line. The first day of pre-training session, the rats were given free access to food for 20 min in a neutral cage. The second day, rats were given free access to the reinforcement for 10 min in the same neutral cage and placed for 10 min in the experimental box without the sponges. Training was carried out in a single session, in five trials. Latency before a correct response (nose-poke into the reinforced sponge) and number of errors (nose-poke into incorrect sponges) were recorded.

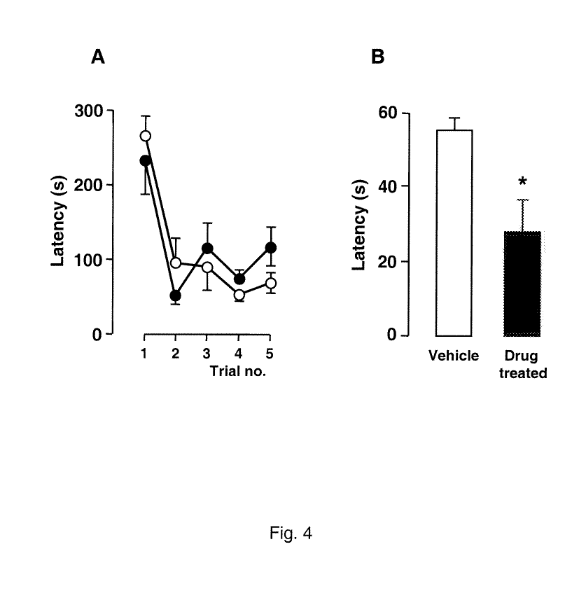

All rats rapidly acquired an association between an odor and a food reward (FIG. 4A). When rats were subsequently administered Mdk into the 3rd cerebral ventricle 2 hours post training, they showed improved odor-reward association recall 96 hours post training (FIG. 4B).

### Example 5

### The Deficit in Prepulse Inhibition is Reversed by ICV Midkine Administration

Prepulse inhibition of startle is used as a readout of sensorimotor processing in the prefrontal cortex. Midkine administration into the 3rd cerebral ventricle of the brain reverses the deficit observed in prepulse inhibition of startle in animals that have been reared in isolation.

Isolation Rearing (Geyer et al., 1993)

Isolation reared animals (n=7-8) were raised in single perspex cages from weaning on postnatal day (P) 25. Separate cohorts were analyzed at P30, P40, P60 and P80. All isolation reared animals are compared to aged matched social controls (raised in cages of 3-4 from weaning, n=8).

Prepulse Inhibition of Startle Response (Ellenbroek et al., 1998)

The startle apparatus consisted of a sound attenuating chamber (54.6×50.8×30.5 cm), a startle platform which measured the startle response, and an audio stimulator, controlled by the startle reflex software (MED Associates Inc.). Animals were restrained in an appropriately sized animal holder, placed on the startle platform. Animals were placed in the apparatus and allowed to acclimatize to a background noise of 70 dB[A] for 5 minutes. Subsequently, the animals received five startle trials (120 bD[A] burst of white noise lasting 20 ms). Next, the rats received five blocks of trials, consisting of two startle trials and four prepulse inhibition trials (of differing prepulse intensity), as well as a no stimulus condition. The prepulse inhibition trials consisted of a prepulse stimulus of 72, 76, 80 or 84 dB[A] burst of white noise, followed 100 ms later by the startle stimulus of 120 dB[A]. The inter-trial interval was between 10 and 20 s. The session terminated with five further startle trials. The resulting movement of the rats was measured during 100 ms after startle stimulus onset, and then rectified, amplified and analyzed by computer and the maximal response and average response over the 100 ms period determined. Baseline startle amplitude was determined as the mean response of all startle trials. The % prepulse inhibition was determined according to the following formula: 100−([startle amplitude at prepulse trial/startle amplitude at startle trial]×100%). The effect on baseline startle amplitude was analyzed by a one-way analysis of variance (ANOVA). The effect of rearing on PPI was determined by 2-way ANOVA (factors: rearing and prepulse intensity), with post-hoc Bonferroni tests.

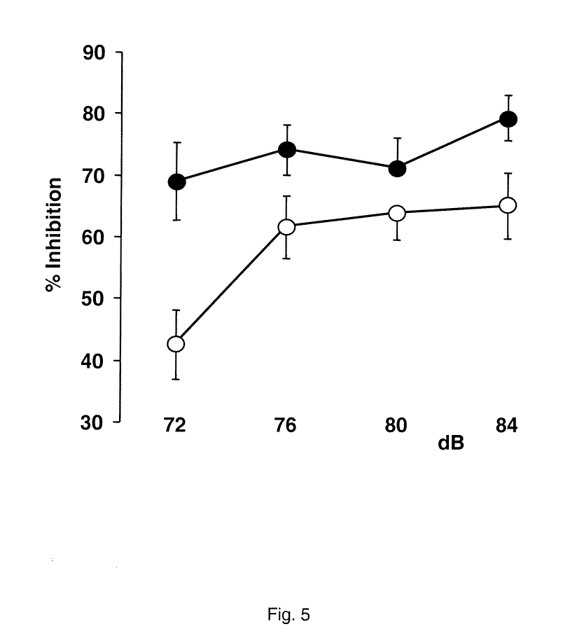

Isolation reared animals exhibited a significant deficit in prepulse inhibition (PPI) of startle. Four daily ICV injections of midkine (2 μg/day) reversed PPI behavior back to normal compared to vehicle-treated animals (Two-way ANOVA, p<0.05 for treatment) (FIG. 5).

### Example 6

### PCR Confirmation of Ttr Regulation

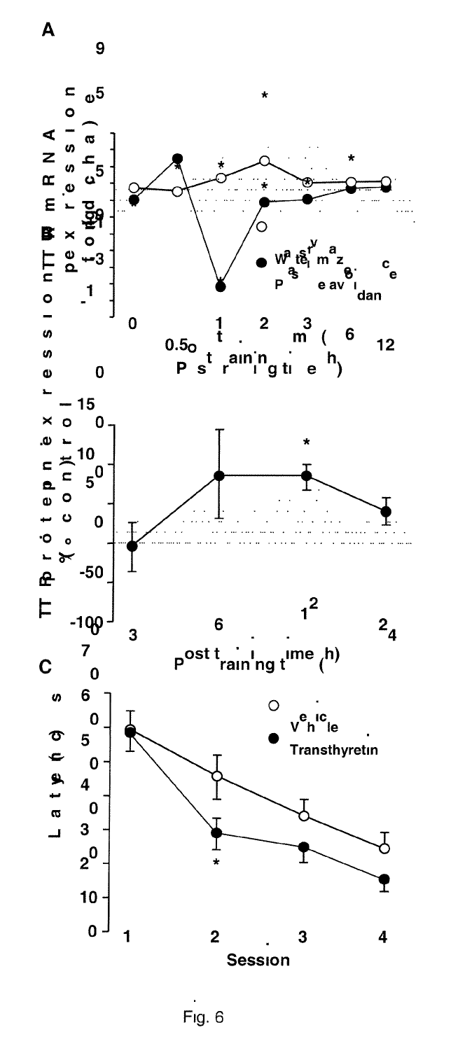

This example provides confirmation of the pattern of Ttr regulation over time following the learning tasks, using quantitative real-time PCR as described above. The temporal pattern of mRNA expression of Ttr was confirmed following water maze training (FIG. 6A), showing an increase at 2 hours, and following passive avoidance learning (FIG. 6A), showing an increase at 30 minutes, a decrease at 1 hour, and returning to basal levels thereafter.

### Example 7

### Protein Level Regulation of Ttr

The regulation of Ttr at message level was shown to translate into a prolonged increase at the protein level following water maze training, which lasted from 6-12 hours (FIG. 6B). Protein samples were prepared and analyzed as described above.

### Example 8

### Intracerebroventricular Administration of Ttr Improves Memory Consolidation

When recombinant Ttr was administered into the 3rd cerebral ventricles of rats, spatial memory was enhanced following training on the Morris Water Maze. Animals were trained on the Morris Water Maze as described above. Ttr improved spatial memory on this learning task when administered to the 3rd cerebral ventricle 2 hours post training on each of the four days of training (FIG. 6C).

### Example 9

### PCR Confirmation of Unknown EST Regulation

This example provides confirmation of the pattern of an unknown EST's regulation over time following the learning tasks, using quantitative real-time PCR as described above.

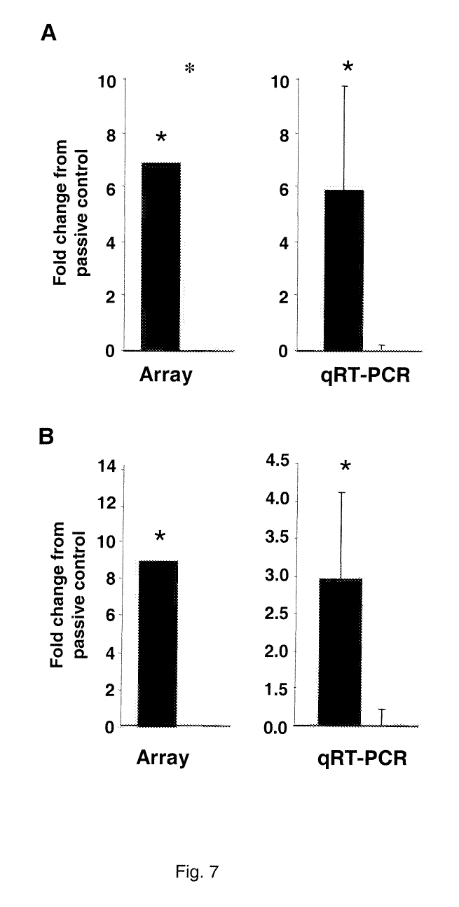

The temporal pattern of mRNA expression of the unknown EST, UK8, was confirmed at the time points of interest following water maze training (FIG. 7A), showing an increase at 2 hours. The temporal pattern of mRNA expression of UK8 was also confirmed at the time points of interest following passive avoidance learning (FIG. 7B), showing an increase at 0.5 hours.

### Example 10

### In Situ Hybridization of Unknown EST

The expression of this unknown transcript was analyzed by in situ hybridization as described below.

In Situ Hybridization

All steps prior to and during hybridization were conducted under RNase-free conditions. 40 μm fresh frozen sections were fixed in cold (4° C.) 4% paraformaldehyde in PBS before being washed in PBS and 0.1% Triton-X. Sections were incubated in a humidified chamber overnight at 60° C. with hybridization solution containing 1% of the digoxigenin-UTP labeled antisense or sense RNA probes. The slides were then washed twice for 15 mM each time with 2×sodium-saline-citrate buffer (SSC), 1×SSC and 0.1×SSC at 60° C. After washing, slides were prepared for immunodetection by incubating them in 150 mM NaCl and 100 mM Tris, pH 7.5 (Buffer 1) containing 1% normal goat serum and 0.1% triton-X for 30 mM at room temperature. The sections were then exposed to anti-digoxigenin alkali-phosphatase conjugate (Boehringer Mannheim) at 1:1000 dilution in the same buffer for 2 h at room temperature. They were then washed twice for 10 min each with Buffer 1 and Buffer 2 (100 mM NaCl, 100 mM Tris-HCl, pH 9.5, and 50 mM MgCl2). The bound antibody was detected by incubating the slides with 5-Bromo-4-Chloro-3-Indolyl Phosphate/Nitroblue Tetrazolium (BCIP/NBT) substrate (Boehringer Mannheim), producing a purple precipitate reaction product. The reaction was stopped by incubating sections in Buffer 3 (10 mM Tris-HCl (pH 8.1) and 1 mM EDTA) and then washed with distilled water. The sections were then mounted in glycerol (Sigma) and imaged using an Aperio scanning system. The Aperio Slide Scanning system at the UCD Conway Institute was funded under a Health Research Board of Ireland Equipment Grant.

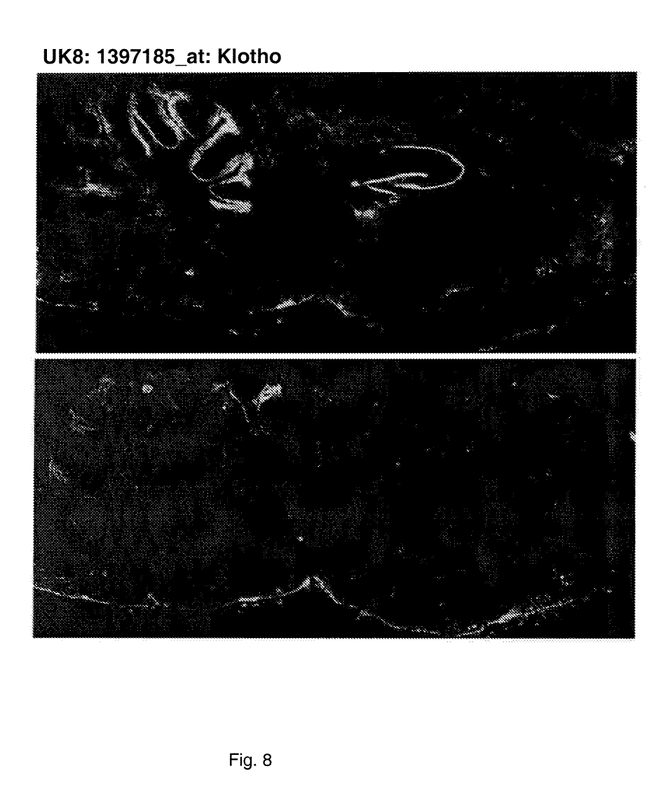

In situ hybridization analysis demonstrated that UK8 was highly localized to the hippocampus and cerebellum (FIG. 8), further confirming a specific role in memory-associated synaptic plasticity. UK8 is expressed in a very discrete manner in the adult rodent brain. Panel A shows high expression of UK8 in the hippocampus and cerebellum using the antisense probe, while no such expression is observed with the sense control probe (Panel B). CA: CA pyramidal cells; DG: dentate gyrus granule cells; CB: cerebellum.

1. A method of identifying gene targets associated with learning, memory, and/or memory consolidation, comprising
(a) assessing transcriptional regulation in a tissue over time in animals following the animals being administered a plurality of learning, memory, and/or memory consolidation tasks, wherein the animals are comprised of at least two groups, wherein each group is administered a different learning, memory, and/or memory consolidation task, and detecting a cohort of transcripts of which expression is regulated in an identifiable temporal pattern following at least two of the learning tasks;
(b) comparing the identifiable temporal patterns of regulated expression from the cohorts detected in (a); and
(c) detecting a cohort of transcripts from (b) wherein said transcripts are temporally regulated in tissue from animals administered a different learning, memory, and/or memory consolidation task, and which cohort comprises transcripts present in (b), which exhibits an identifiable temporal pattern of regulated expression following at least two of the learning tasks.

2. The method of claim 1, wherein the animals are administered two learning tasks, each task being administered to a separate group of animals.

3. The method of claim 1 wherein the learning and memory tasks comprise the Morris water maze and a passive avoidance test.

4. The method of claim 1 wherein the transcripts comprise an expressed sequence tag.

5. The method of claim 1, wherein the tissue is brain tissue.

6. The method of claim 1 wherein the expression is regulated within 24 hours of at least one learning, memory, and/or memory consolidation tasks.

7. The method of claim 1 wherein the expression is regulated within 1 week of at least one of the learning, memory, and/or memory consolidation tasks.

8. The method of claim 1 wherein the expression is regulated within 1 month of at least one of the learning, memory, and/or memory consolidation tasks.

9. The method of claim 1 wherein one or more of the transcripts within the cohort detected in (c) was previously identified as associated with learning, memory, and/or memory consolidation.

10. The method of claim 1 wherein the cohort detected in (c) comprises midkine.

11. The method of claim 1 wherein the cohort detected in (c) comprises transthyretin.

12. The method of claim 1 wherein the transcript in unknown.

13. The method of claim 1 further comprising a change in transcript expression within the cohort detected in (c) that is 2-fold.

14. The method of claim 1 further comprising a change in transcript expression within the cohort detected in (c) that is 10-fold.

15. The method of claim 1 further comprising a change in transcript expression within the cohort detected in (c) is 100-fold.

16. An isolated nucleic acid molecule comprising at least one transcript identified from the method of claim 1.

17. The isolated nucleic acid molecule of claim 16 comprising a transcript selected from the group consisting of SEQ ID NO:1 through SEQ ID NO:11.

18. A method of modulating memory function in an animal comprising enhancing the gene expression product of at least one transcript identified from the method of claim 1.

19. The method of claim 18 comprising enhancing the gene expression product of at least one transcript selected from the group consisting of SEQ ID NO:1 through SEQ ID NO:11.

20. A method of modulating memory function in an animal comprising enhancing the gene expression of at least one transcript identified from the method of claim 1.

21. The method of claim 20 comprising enhancing the gene expression of at least one transcript selected from the group consisting of SEQ ID NO:1 through SEQ ID NO:11.

22. A method of modulating memory function in an animal comprising reducing the gene expression product of at least one transcript identified from the method of claim 1.

23. The method of claim 22 comprising reducing the gene expression product of at least one transcript selected from the group consisting of SEQ ID NO:1 through SEQ ID NO:11.

24. A method of modulating memory function in an animal comprising downregulating the gene expression of at least one transcript identified from the method of claim 1.

25. The method of claim 24 comprising downregulating the gene expression of at least one transcript selected from the group consisting of SEQ ID NO:1 through SEQ ID NO:11.

26. A method of diagnosing a memory disorder in an animal comprising
(a) determining the gene expression profile in normal animals of at least one transcript identified from the method of claim 1 following a learning, memory, and/or memory consolidation task;
(b) determining the gene expression profile in test animals of at least one transcript identified from the method of claim 1 following a learning, memory, and/or memory consolidation task; and
(c) determining which nucleic acid sequences show an abnormal expression profile through a comparison of the profiles obtained in (a) and (b).

27. The method of claim 26, wherein the at least one transcript is selected from the group consisting of SEQ ID NO:1 through SEQ ID NO:11.

28. A method of identifying a compound that modulates memory function comprising
(a) administering a test compound to a first group of animals;
(b) administering a learning and memory task to said first group of animals
(c) administering a placebo to a second group of animals;
(d) administering a learning, memory, and/or memory consolidation task to said second group of animals
(e) assessing the gene expression profiles of at least one transcript identified from the method of claim 1, following said tasks in the animals from (a) and (c); and
(f) determining if one or more of the nucleic acid sequences from (e) exhibits any detectable change in activity when comparing the animals administered the placebo to the animals administered the test compound.

29. The method of claim 28, wherein the at least one transcript is selected from the group consisting of SEQ ID NO:1 through SEQ ID NO:11.
